SEQUENCE LISTING (13 sequences)

SEQ ID NO: 1 - DNA, 44 bp, synthetic construct
  source 1..44: mol_type = other DNA
cgacgccagtttgaaggttcgttcgcaggtgtggagtgacgtcg

SEQ ID NO: 2 - DNA, 44 bp, synthetic construct
  source 1..44: mol_type = other DNA
cgactggtaggcagataggggaagctgattcgatgcgtgggtcg

SEQ ID NO: 3 - RNA, 38 nt, synthetic construct
  source 1..38: mol_type = other RNA
agtgataccagcatcgtcttgatgcccttggcagcact

SEQ ID NO: 4 - protein, 190 aa, synthetic construct
GLDNELSLVDGQDRTLTVQQWDTFLNGVFPLDRNRLTREWFHSGRAKYIVAGPGADEFEGTLELGYQIGFPWSLGVGINFSYTTPNILINNGNITAPPFGLNSVITPNLFPGVSISANLGNGPGIQEVATFSVNVSGAKGGVAVSNAHGTVTGAAGGVLLRPFARLIASTGDSVTTYGEPWNMNHHHHHH

SEQ ID NO: 5 - protein, 190 aa, synthetic construct
GLDNELSLVDGQDRTLTVQQWDTFLNGVFPLDRNRLTREWFHSGRAKYIVAGPGADEFEGTLELGYQIGFPWSLGVGINFSYTTPNILINNGNITAPPFGLNSVITPNLFPGVSISARLGNGPGIQEVATFSVNVSGAKGGVAVSNAHGTVTGAAGGVLLRPFARLIASTGDSVTTYGEPWNMNHHHHHH

SEQ ID NO: 6 - protein, 190 aa, synthetic construct
GLDNELSLVDGQDRTLTVQQWDTFLNGVFPLDRNRLTREWFHSGRAKYIVAGPGADEFEGTLELGYQIGFPWSLGVGINFSYTTPNILINNGNITAPPFGLNSVITPNLFPGVSISANLGNGPGIQEVATFSVRVSGAKGGVAVSNAHGTVTGAAGGVLLRPFARLIASTGDSVTTYGEPWNMNHHHHHH

SEQ ID NO: 7 - protein, 190 aa, synthetic construct
GLDNELSLVDGQDRTLTVQQWDTFLNGVFPLDRNRLTREWFHSGRAKYIVAGPGADEFEGTLELGYQIGFPWSLGVGINFSYTTPNILINNGNITAPPFGLNSVITPNLFPGVSISARLGNGPGIQEVATFSVRVSGAKGGVAVSNAHGTVTGAAGGVLLRPFARLIASTGDSVTTYGEPWNMNHHHHHH

SEQ ID NO: 8 - protein, 190 aa, synthetic construct
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGAEGGVAVSNAHGTVTGAAGGVLLRPFARLIASTGDSVTTYGEPWNMNHHHHHH

SEQ ID NO: 9 - DNA, 20 bp, synthetic construct
  source 1..20: mol_type = other DNA
cgacgccagtagtgacgtcg

SEQ ID NO: 10 - DNA, 32 bp, synthetic construct
  source 1..32: mol_type = other DNA
cgacgccagtttgcaggtgtggagtgacgtcg

SEQ ID NO: 11 - DNA, 36 bp, synthetic construct
  source 1..36: mol_type = other DNA
cgacgccagtttgaaggttcgttcggagtgacgtcg

SEQ ID NO: 12 - DNA, 44 bp, synthetic construct
  source 1..44: mol_type = other DNA
cgacgccagtttgaagattcgttcgcagatgtgaagtgacgtcg

SEQ ID NO: 13 - DNA, 28 bp, synthetic construct
  source 1..28: mol_type = other DNA
cctttgagttcccggcctaaccgctggc

(19) United States
(12) Patent Application Publication
(10) Pub. No.: US 2026/0348940 A1
(43) Pub. Date: Nov. 5, 2026

(54) A NANOPORE-BASED NUCLEIC ACID CONFORMATION READER FOR SMALL MOLECULE (E.G. NEUROTRANSMITTERS AND HORMONES) BIOSENSING AND THERAPEUTICAL COMPOUND SCREENING

(71) Applicant: THE CURATORS OF THE UNIVERSITY OF MISSOURI, Columbia, MO (US)

(72) Inventors: Li-Qun GU, Columbia, MO (US); Kai TIAN, Pleasanton, CA (US); Yin-Tzu CHEN, Columbia, MO (US)

(21) Appl. No.: 19/486,845

(22) PCT Filed: May 21, 2024
(86) PCT No.: PCT/US2024/030380

§ 371 Date: Nov. 21, 2025

Related U.S. Application Data
(60) Provisional application No. 63/468,133, filed on May 22, 2023.

(51) Int. Cl. G01N 33/487; C12Q 1/6869

(52) U.S. Cl. CPC G01N 33/48721; C12Q 1/6869

(57) ABSTRACT

Provided herein are real-time, label-free methods, systems, apparatuses, and compositions for the detection and characterization of dynamic aptamer-small molecule interactions through the use of nanopore systems that pro-artificial riboswitch vide accurate detection of time-resolved dynamic nucleic acid conformational variations in response to small molecule binding. The methods, systems, apparatuses, and compositions can quantify specific ligands, elucidate nucleic acid-ligand interactions, and pinpoint the nucleic acid motifs for ligand binding, showing the potential for small molecule biosensing, drug discovery assayed via RNA and DNA conformation changes, and the design of artificial riboswitch effectors in synthetic biology.

### CROSS-REFERENCE TO RELATED APPLICATIONS

This application claims priority under 35 U.S.C. § 119 to provisional application Ser. No. 63/468,133 filed May 22, 2023 titled A Nanopore-Based Nucleic Acid Conformation Reader For Small Molecule (E.G. Neurotransmitters And Hormones) Biosensing And Therapeutical Compound Screening, herein incorporated by reference in its entirety including, without limitation, the specification, claims, and abstract, as well as any figures, tables, or drawings thereof.

### STATEMENT REGARDING FEDERALLY SPONSORED RESEARCH

This invention was made with government support under GM114204, and HG009338 awarded by the National Institutes of Health. The government has certain rights in the invention.

### INCORPORATION OF SEQUENCE LISTING

The instant application contains a sequence listing, which has been submitted in XML file format by electronic submission and is hereby incorporated by reference in its entirety. Said XML file, created on May 14, 2024, is named P14706WO00.xml and is 13,354 bytes in size.

### TECHNICAL FIELD

The present disclosure relates generally to fast, low-cost, accurate, label-free detection of time-resolved dynamic nucleic acid conformational variation in response to small molecule binding useful in improving knowledge of nucleic acid structures and regulation of structure by small molecules as a key mechanism in their biological functions, the discovery of small molecule drug compounds that target nucleic acid structures as a therapeutic strategy, the development of new tools for synthetic biology based on gene expression networks regulated by small molecules; and the development of new sensors of small molecules such as neurotransmitters for studies in neurochemistry and disease diagnostics.

### BACKGROUND

Nucleic acids can undergo conformational changes upon binding small molecules. These conformational changes can be exploited to develop new therapeutic strategies through control of gene expression or triggering of cellular responses and can also be used to develop sensors for small molecules such as neurotransmitters. Many analytical approaches can detect dynamic conformational change of nucleic acids, but they need labeling, are expensive, and have limited time resolution.

One solution to this problem is the use of nanomaterials, in particular nanopores. The nanopore approach can provide a conformational snapshot for each nucleic acid molecule detected. However, existing nanopore technology is unable to detect dynamic nucleic acid conformational change in response to small molecule binding.

There is therefore a need for a modular, label-free, nucleic acid-inlaid nanopore capable of revealing time-resolved, small molecule-induced, single nucleic acid molecule conformational transitions with millisecond resolution.

There is also a need for a nanopore solution that can quantify specific ligands such as neurotransmitters, elucidate nucleic acid-ligand interactions, and pinpoint the nucleic acid motifs for ligand binding, thereby enabling small molecule biosensing, drug discovery assayed via RNA and DNA conformational changes, and the design of artificial riboswitch effectors in synthetic biology.

These and other objects, advantages, and features of the present disclosure will become apparent from the following specification taken in conjunction with the claims set forth herein.

### SUMMARY

The present disclosure relates to methods, systems, apparatuses and compositions for making and using nucleic acid-docked nanopores. In particular, the disclosure provides for a nucleic acid-docked nanopore system comprising: (a) a phospholipid membrane; (b) a mutant Mycobacterium smegmatis porin A (MspA) nanopore transversing the membrane and comprising a cis vestibule and a trans vestibule, wherein the cis vestibule comprises a lumen and the trans vestibule comprises a constriction; wherein the mutant MspA comprises a mutation at positions 90, 91, 93, 118, 134, and/or 139, wherein a negatively charged amino acid at positions 90, 91, 93, 118, 134, and/or 139 of a wild-type MspA is replaced by a positively charged amino acid or a neutral polar amino acid such that the one or more positively charged amino acids or a neutral polar amino acids are distributed around the interior circumference of the mutant MspA nanopore thereby forming a ring; (c) an aptamer non-covalently bound with the ring on the interior surface of the mutant MspA nanopore such that the aptamer is suspended in the lumen via non-covalent bonding between the aptamer and the nanopore; wherein non-covalent binding suspends the aptamer in or near the focus of the ring or in any position in the ring; (d) a ligand capable of binding with the aptamer; (e) an electrical circuit capable of applying an electric field to the nanopore system and generating an ion current, wherein one or more measurements are taken of the ion current; wherein the one or more measurements are indicative of one or more characteristics of the aptamer, the ligand, an interaction between the aptamer and the ligand, or an interaction between the aptamer and the nanopore; wherein the nanopore system provides real-time characterization of the aptamer, the ligand, the interaction between the aptamer and the ligand, or the interaction between the aptamer and the nanopore; and wherein the nanopore system is adapter-free.

In an embodiment, the nucleic acid-docked nanopore system further comprises (f) a cis chamber located on the side of the membrane closest to the cis vestibule; a (g) a trans chamber located the side of the membrane closest to the trans vestibule; and/or (h) an apparatus capable of taking one or more measurements of the ion current.

In an embodiment, the mutant MspA comprises a mutant as shown in one or more of SEQ ID NO: 7, SEQ ID NO: 4, SEQ ID NO:6, and SEQ ID NO: 5.

According to an embodiment, the ligand comprises a neurotransmitter, antibody, receptor, peptide, nucleic acid sequence, hormone, metabolite, antibiotic, therapeutic compound, biomarker, and/or diagnostic compound.

In an embodiment, the aptamer comprises a sequence of DNA, RNA, or XNA, a peptide, oligonucleotide, a riboswitch aptamer, an RNA element, an RNA structure, an RNA entry site, a frameshifting element, and/or RNA repeats.

According to some embodiments, the aptamer is an oligonucleotide having the sequence shown in SEQ ID NO:1 and has the ability to bind with a ligand comprising dopamine; the aptamer is an oligonucleotide having the sequence shown in SEQ ID NO:2 and has the ability to bind with a ligand comprising serotonin; and/or the aptamer is an oligonucleotide having the sequence shown in SEQ ID NO:3 and has the ability to bind with a ligand comprising theophylline.

The disclosure also relates to methods of non-covalently docking an aptamer in a nucleic acid-docked nanopore comprising: (a) providing a mutant Mycobacterium smegmatis porin A (MspA) nanopore comprising a cis vestibule comprising a lumen and a trans vestibule comprising a constriction that define a channel, wherein the mutant MspA comprises a mutation at positions 90, 91, 93, 118, 134, and/or 139, wherein a negatively charged amino acid at positions 90, 91, 93, 118, 134, and/or 139 of a wild-type MspA is replaced by a positively charged amino acid or a neutral polar amino acid such that the one or more positively charged amino acids or a neutral polar amino acids are distributed around the interior circumference of the mutant MspA nanopore thereby forming a ring; (b) delivering an aptamer to the mutant MspA nanopore; and (c) non-covalently binding the aptamer with the ring on the interior surface of the mutant MspA nanopore, wherein non-covalent binding suspends the aptamer in or near the focus of the ring or in any position in the ring; wherein neither the nanopore or aptamer are modified with an adapter and wherein the aptamer is capable of binding with a ligand that is not modified with an adapter.

In an embodiment of the methods, the mutant MspA comprises a mutant as shown in one or more of SEQ ID NO: 7, SEQ ID NO: 4, SEQ ID NO:6, and SEQ ID NO: 5.

According to an embodiment, the aptamer comprises a sequence of DNA, RNA, or XNA, a peptide, oligonucleotide, a riboswitch aptamer, an RNA element, an RNA structure, an RNA entry site, a frameshifting element, and/or RNA repeats.

In a further embodiment, the aptamer is an oligonucleotide comprising a nucleobase sequence according to any one or more of SEQ ID NO: 1, SEQ ID NO:2, and SEQ ID NO: 3.

The disclosure also relates to methods of ligand characterization comprising: (a) providing an inlaid nucleic acid nanopore system comprising (i) a phospholipid membrane; (ii) a mutant Mycobacterium smegmatis porin A (MspA) nanopore transversing the membrane and comprising a cis vestibule and a trans vestibule, wherein the cis vestibule comprises a lumen and the trans vestibule comprises a constriction; wherein the mutant MspA comprises a mutation at positions 90, 91, 93, 118, 134, and/or 139, wherein a negatively charged amino acid at positions 90, 91, 93, 118, 134, and/or 139 of a wild-type MspA is replaced by a positively charged amino acid or a neutral polar amino acid such that the one or more positively charged amino acids or a neutral polar amino acids are distributed around the interior circumference of the mutant MspA nanopore thereby forming a ring; (iii) an aptamer non-covalently bound with the ring on the interior surface of the mutant MspA nanopore such that the aptamer is suspended in the lumen via non-covalent bonding between the aptamer and the nanopore; wherein non-covalent binding suspends the aptamer in or near the focus of the ring or in any position in the ring; (iv) a ligand capable of binding with the aptamer; (v) an electrical circuit capable of applying an electric field; wherein the nanopore system is adapter-free; (b) applying an electric field to the nanopore system using the electrical circuit, thereby generating an ion current; (c) delivering the ligand to the nanopore such that the ligand moves into the pore; (d) allowing the ligand to bind and/or unbind with the aptamer; and (e) taking one or more measurements of the ion current; wherein the one or more measurements are indicative of one or more characteristics of the aptamer, the ligand, an interaction between the aptamer and the ligand, or an interaction between the aptamer and the nanopore; and wherein the nanopore system provides real-time characterization of the aptamer, the ligand, the interaction between the aptamer and the ligand, or the interaction between the aptamer and the nanopore.

According to an embodiment, the one or more characteristics comprise the length of the ligand, the identity of the ligand, the sequence of the ligand, the presence of the ligand, the absence of the ligand, the secondary structure of the ligand; whether or not the ligand is modified, the conformation of the aptamer, the dwell time of the ligand; the blocking level of the aptamer, the blocking duration, the block occurrence; or a combination thereof.

In an embodiment, the length of the ligand is measured by sequencing the ligand.

In a further embodiment, the sequencing comprises measuring the ion current generated by the electric field as each unit of the ligand individually binds and unbinds with the aptamer to provide measurable blockade in a current pattern that is associated with each unit; and comparing the current pattern to a current pattern of a known unit obtained under the same conditions, such that the ligand is sequenced.

In an embodiment, the aptamer is an aptamer variant comprising a motif; and the motif comprises a hairpin, a single-branched loop, a multi-branched loop, a helix, a bulge, or a G-quadruplex.

In an embodiment, the one or more characteristics comprises the blocking level of the aptamer.

According to some embodiments, the method further comprises aptamer variant screening; and the aptamer variant screening occurs by repeating the method one or more times with a different aptamer variant.

In an embodiment, the one or more characteristics comprises the presence of a long-duration, single-level blocking event and/or the absence of a long-duration, single-level blocking event; and the presence of a long-duration, single-level block event is indicative of a stable aptamer-ligand interaction.

In an embodiment, the presence of the ligand is indicative of a phenotype or with a type of cell.

In an embodiment, the phenotype comprises a disease or a medical condition; and the cell comprises a bacterium, a virus, a fungus, or a parasite.

These and/or other objects, features, advantages, aspects, and/or embodiments will become apparent to those skilled in the art after reviewing the following brief and detailed descriptions of the drawings. The present disclosure encompasses both combinations of disclosed aspects and/or embodiments and/or reasonable modifications not shown or described.

### BRIEF DESCRIPTION OF THE FIGURES

FIG. 1 shows the principle and applications of a nanopore sensor capable of discriminating nucleic acids conformational transitions in response to the small-molecule binding.

FIG. 2A shows the discrimination of dopamine-induced aptamer conformational changes in an MspA protein nanopore, in particular single-pore current recordings at 180 mV showing signature blocks for aptamer captured from the cis side in the absence of ligands in the presence of dopamine in different concentrations.

FIG. 2B shows the discrimination of dopamine-induced aptamer conformational changes in an MspA protein nanopore, in particular, single-pore current recordings at 180 mV showing signature blocks for aptamer captured from the cis side in the absence of ligands the presence of non-target serotonin or norepinephrine.

FIG. 2C shows the discrimination of dopamine-induced aptamer conformational changes in an MspA protein nanopore on the trans side of the nanopore. Ligand binding events are marked with red lines for dopamine and empty invert triangles for serotonin and norepinephrine. Expanded signatures, current amplitude histograms and proposed kinetic pathway are shown for identifying different aptamer conformations and their transition mechanisms. Greyed intervals depict the unstable intermediate states, Arnt, that occur during transitions between the A₁ and A₂ states.

FIG. 2D shows aptamer residence time τ_{A} in the absence dopamine at different voltages and in the presence of dopamine at optimum 180 mV.

FIG. 2E shows the frequency f of the AL blocks in different dopamine concentrations.

FIG. 2F shows the duration toff of the AL blocks in different dopamine concentrations.

FIG. 2G shows the frequency f of the AL blocks different voltages between 150-180 mV.

FIG. 2H shows the duration tor of the AL blocks in different voltages between 150-180 mV.

FIG. 21 shows the frequency f for binding of the dopamine aptamer with dopamine (25 μM), serotonin (50 μM), and norepinephrine (50 μM). The nanopore was recorded in 1 M KCl and 10 mM Tris (pH7.4), with 100 nM aptamer in cis solution and different concentrations of ligand in the trans solution.

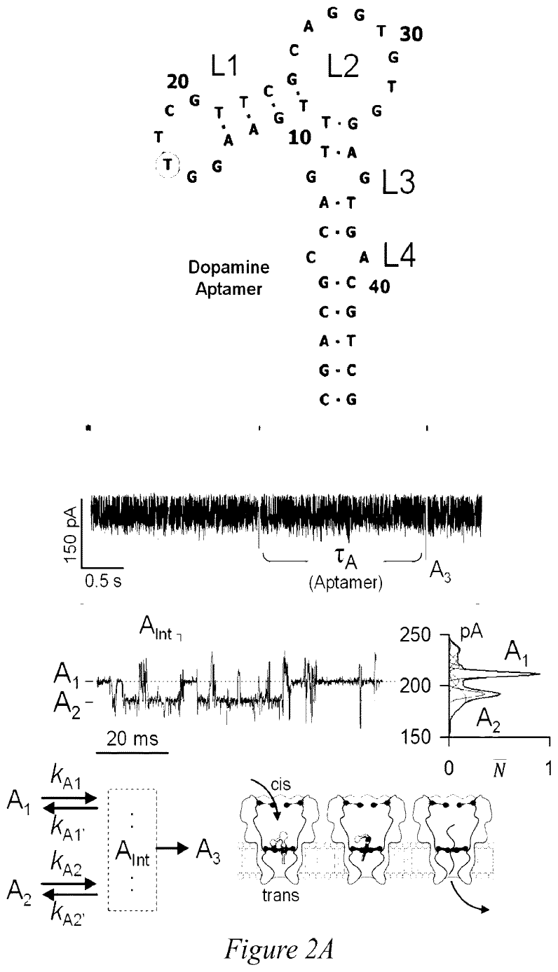

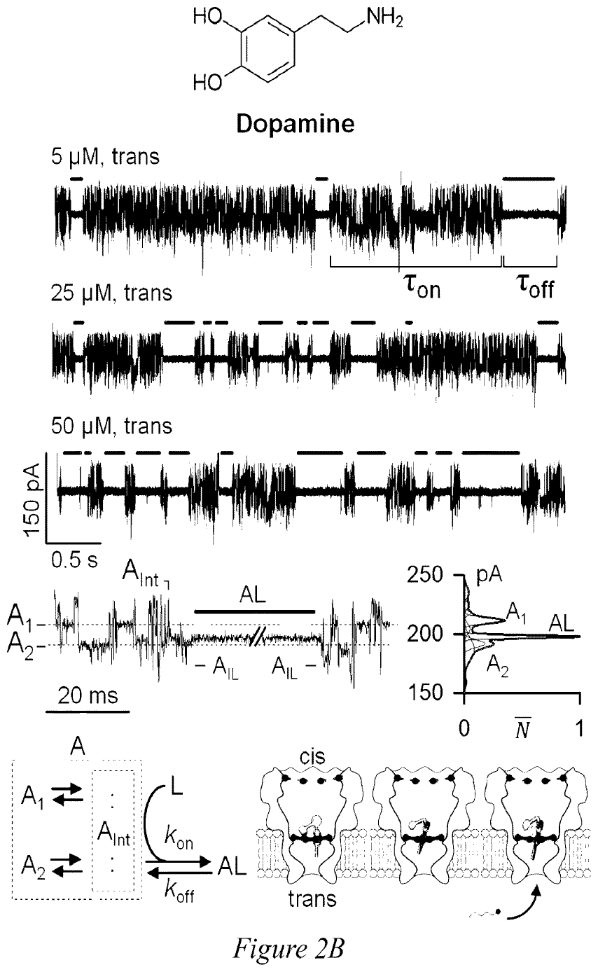

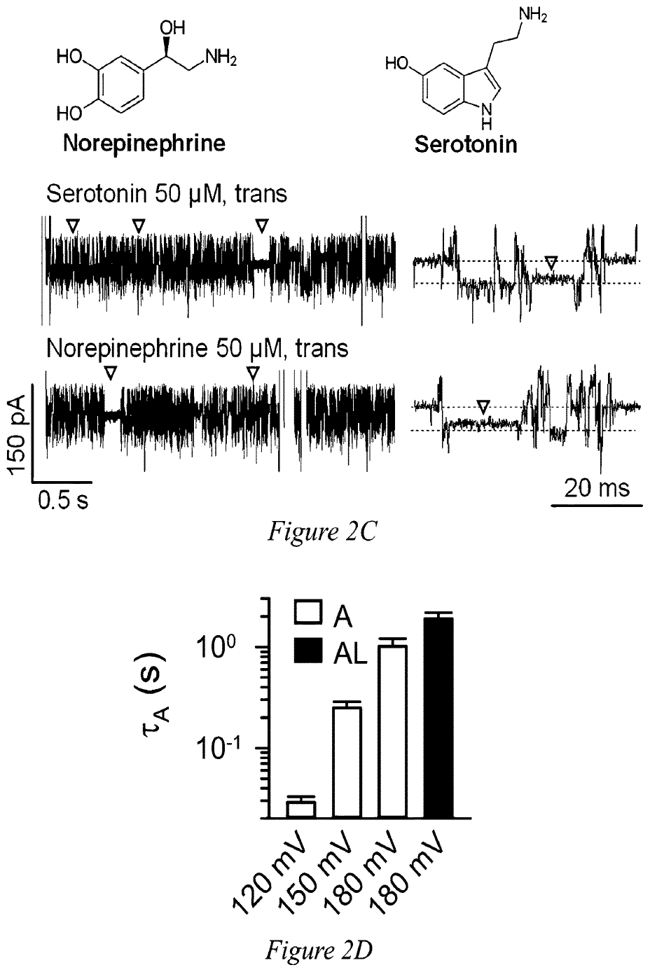

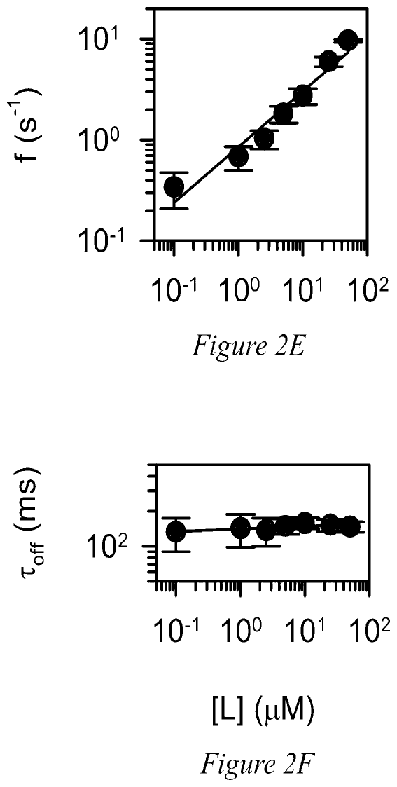

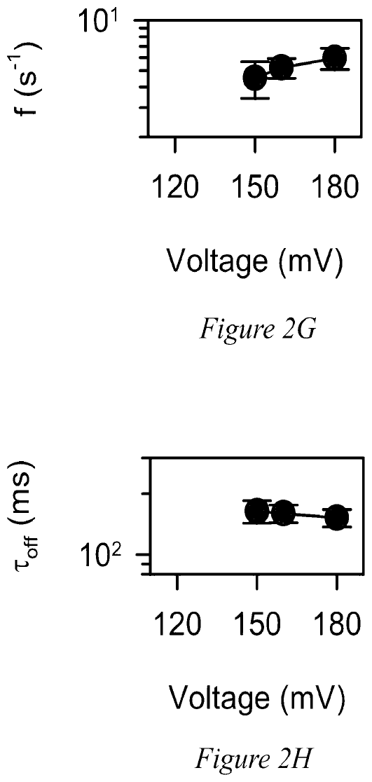

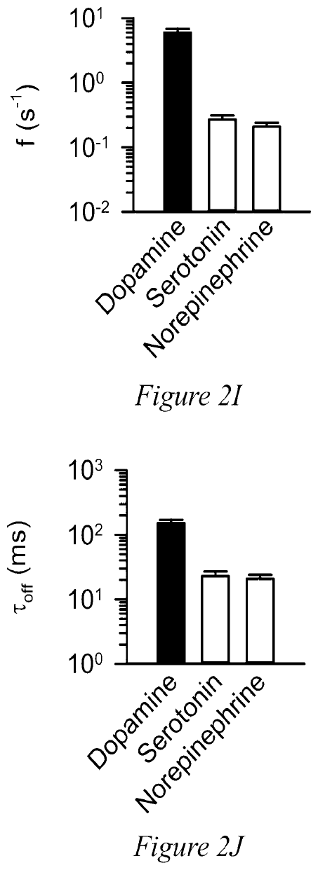

FIG. 2J shows the τ_{off} for binding of the dopamine aptamer with dopamine (25 μM), serotonin (50 μM), and norepinephrine (50 μM). The nanopore was recorded in 1 M KCl and 10 mM Tris (pH7.4), with 100 nM aptamer in cis solution and different concentrations of ligand in the trans solution.

FIG. 3A shows the identification of dopamine binding motifs by screening aptamer variants, in particular the dopamine aptamer variants: ΔL1/L2 (LI and L2 deleted), along with its single-pore currents in the absence (left) and presence (right) of dopamine. The nanopore was recorded at+180 mV in 1 M KCl and 10 mM Tris (pH7.4), with 100 nM aptamer variants in the cis solution and 25 μM concentrations of dopamine in the trans solution.

FIG. 3B shows the identification of dopamine binding motifs by screening aptamer variants, in particular the dopamine aptamer variants: ΔL1 (LI deleted), along with its single-pore currents in the absence (left) and presence (right) of dopamine. The nanopore was recorded at+180 mV in 1 M KCl and 10 mM Tris (pH7.4), with 100 nM aptamer variants in the cis solution and 25 μM concentrations of dopamine in the trans solution.

FIG. 3C shows the identification of dopamine binding motifs by screening aptamer variants, in particular the dopamine aptamer variants: ΔL2 (L2 deleted), along with its single-pore currents in the absence (left) and presence (right) of dopamine. The nanopore was recorded at+180 mV in 1 M KCl and 10 mM Tris (pH7.4), with 100 nM aptamer variants in the cis solution and 25 μM concentrations of dopamine in the trans solution.

FIG. 3D shows the identification of dopamine binding motifs by screening aptamer variants, in particular the dopamine aptamer variants: GG>GA (G substituted with A), along with its single-pore currents in the absence (left) and presence (right) of dopamine. The nanopore was recorded at+180 mV in 1 M KCl and 10 mM Tris (pH7.4), with 100 nM aptamer variants in the cis solution and 25 μM concentrations of dopamine in the trans solution.

FIG. 3E comprises a model showing a conformational mechanism for dopamine binding to the aptamer.

FIG. 4A shows the structure of the MspA-M2 protein nanopore. Positively (black) and negatively (dark gray) charged amino acid residues in the lumen are marked. The aptamer docking site at the R118 ring is highlighted.

FIG. 4B shows charge-altering mutations in the M2 nanopore that were made, including M2-R118N/R134N and corresponding single-nanopore current signatures for the dopamine aptamer in the absence (left) and presence (right) of dopamine. The nanopore was recorded at +180 mV in 1 M KCl and 10 mM Tris (pH7.4), with 100 nM aptamer variants in the cis solution and 25 μM dopamine in the trans solution.

FIG. 4C shows charge-altering mutations in the M2 nanopore that were made, including M2-R118N, and corresponding single-nanopore current signatures for the dopamine aptamer in the absence (left) and presence (right) of dopamine. The nanopore was recorded at +180 mV in 1 M KCl and 10 mM Tris (pH7.4), with 100 nM aptamer variants in the cis solution and 25 μM dopamine in the trans solution.

FIG. 4D shows charge-altering mutations in the M2 nanopore that were made, including M2-R134N, and corresponding single-nanopore current signatures for the dopamine aptamer in the absence (left) and presence (right) of dopamine. The nanopore was recorded at +180 mV in 1 M KCl and 10 mM Tris (pH7.4), with 100 nM aptamer variants in the cis solution and 25 μM dopamine in the trans solution.

FIG. 5A depicts a characterization of serotonin-binding aptamer and theophylline riboswitch aptamer conformational change upon ligand binding in the nanopore, in particular single nanopore current recordings at 120 mV showing signature blocks for the serotonin aptamer alone.

FIG. 5B depicts a characterization of serotonin-binding aptamer and theophylline riboswitch aptamer conformational change upon ligand binding in the nanopore, in particular single nanopore current recordings at 120 mV showing signature blocks for the serotonin aptamer in the presence of 25 μM and 50 μM serotonin.

FIG. 5C depicts a characterization of serotonin-binding aptamer and theophylline riboswitch aptamer conformational change upon ligand binding in the nanopore, in particular single nanopore current recordings at 120 mV showing signature blocks for 50 μM dopamine in the trans solution.

FIG. 5D depicts a characterization of serotonin-binding aptamer and theophylline riboswitch aptamer conformational change upon ligand binding in the nanopore, in particular single nanopore current recordings at 120 mV showing signature blocks for the theophylline aptamer alone.

FIG. 5E depicts a characterization of serotonin-binding aptamer and theophylline riboswitch aptamer conformational change upon ligand binding in the nanopore, in particular single nanopore current recordings at 120 mV showing signature for the theophylline aptamer in 25 μM theophylline in the trans solution.

FIG. 5F shows the kinetic scheme for ligands binding to the serotonin and theophylline aptamers.

FIG. 5G shows the residence time (τ_{A}) of the serotonin aptamer in the absence ligand at different voltages, and in the presence of 25 μM ligand at 120 mV.

FIG. 5H shows the residence time (τ_{A}) of the theophylline aptamer in the absence ligand at different voltages, and in the presence of 25 μM ligand at 180 mV.

FIG. 5I shows the frequency f of the serotonin and theophylline binding blocks with different ligand concentrations.

FIG. 5J shows the τ_{off} of the serotonin and theophylline binding blocks with different ligand concentrations.

FIG. 5K shows the frequency f of the serotonin and theophylline binding blocks at different voltages.

FIG. 5L shows the τ_{off} of the serotonin and theophylline binding blocks at different voltages.

FIG. 5M shows the frequency f for binding of the serotonin aptamer with 25 μM serotonin or 50 μM dopamine at 120 mV.

FIG. 5N shows the τ_{off} for binding of the serotonin aptamer with 25 μM serotonin or 50 μM dopamine at 120 mV.

FIG. 6A provides a comparison of nanopore ion current standard deviations (I_{SD}) for the dopamine-, serotonin- and theophylline-binding aptamers in conformations without and with ligand binding. For the dopamine aptamer, the reduction in current fluctuations is primarily due to the stabilization of the aptamer such that it no longer fluctuates between multiple conformations including A₁ and A₂. On the other hand, fluctuations are also reduced upon binding of serotonin or theophylline to their respective aptamers, even though only a single main current-blocking conformation is resolved before ligand binding. This is presumably because the aptamer assumes a more stable configuration in the nanopore upon ligand binding.

FIG. 6B provides a comparison of nanopore ion current blocking levels (I/I₀, b) for the dopamine-, serotonin- and theophylline-binding aptamers in conformations without and with ligand binding.

FIG. 7A shows expanded single-pore current recordings revealing various dopamine-binding aptamer conformation states and their dynamic transitions, in particular recordings hardware-filtered at 5 kHz, wherein dashed lines mark the distinct blocking levels for the aptamer's conformation states A₁ and A₂ and greyed intervals depict the fluctuating intermediate states A_{Int}.

FIG. 7B shows expanded single-pore current recordings revealing various dopamine-binding aptamer conformation states and their dynamic transitions, in particular recording hardware-filtered at 100 kHz (upper trace) and then software-filtered at 20 kHz (lower trace), wherein dashed lines mark the distinct blocking levels for the aptamer's conformation states A₁ and A₂ and greyed intervals depict the fluctuating intermediate states Δ_{Int}.

FIG. 7C shows expanded single-pore current recordings revealing various dopamine-binding aptamer conformation states and their dynamic transitions, wherein transient, highly blocking state A₃ marks the termination of the aptamer signature. Without being bound by theory, it is thought that A₃ represents transient disruption of the aptamer structure followed by translocation through the nanopore to the trans solution, whereas in signatures without terminal A₃, the aptamer returns to the cis solution before unfolding. 73% of signatures generate A₃ from an intermediate cluster A_{Int} (i), only 4% from A₂ (ii), 0% from A₁ (iii), and 23% without A₃ (iv). This indicates that the aptamer rarely unfolds from an A₁ or A₂ conformation, consistent with their higher stability compared to the intermediate states.

FIG. 7D is a histogram of A₃ duration (τ_{A3}). A₃ has a mean lifetime τ_{A3}=0.39 ms, thus is resolvable at a bandwidth of 5 kHz.

FIG. 8A shows expanded single-pore current recordings revealing dynamic transitions between free and dopamine-bound aptamer conformations, wherein recordings are hardware-filtered at 5 kHz, wherein recordings are hardware-filtered at 100 kHz (upper trace) and then software-filtered at 20 kHz (lower trace). Dashed lines mark the distinct blocking levels for the aptamer's conformation states A₁ and A₂ and greyed intervals denote clusters of the unstable intermediate A_{Int}. Dopamine-bound conformations AL are marked by black bars, which are generated from A_{Int} states with blocking levels lower than A₂ and return to similar states upon dopamine dissociation from the aptamer.

FIG. 8B shows expanded single-pore current recordings revealing dynamic transitions between free and dopamine-bound aptamer conformations, wherein recordings are hardware-filtered at 100 kHz (upper trace) and then software-filtered at 20 kHz (lower trace). Dashed lines mark the distinct blocking levels for the aptamer's conformation states A₁ and A₂ and greyed intervals denote clusters of the unstable intermediate A_{Int}. Dopamine-bound conformations AL are marked by black bars, which are generated from A_{Int} states with blocking levels lower than A₂ and return to similar states upon dopamine dissociation from the aptamer.

FIG. 8C shows expanded single-pore current recordings revealing dynamic transitions between free and dopamine-bound aptamer conformations, in particular blocking transition A₃ at the aptamer signature terminal and its characterization. Without being bound by theory, it is thought that A₃ represents a process of aptamer unfolding followed by nanopore translocation, whereas a signature without terminal A₃ indicates the return of the aptamer to the cis solution without nanopore translocation. 19% of aptamer signatures generate A₃ from an A_{Int} state (i), 0% from the dopamine-bound conformation AL (ii), and 81% without A₃ (iii) This A₃ property suggests that dopamine binding stabilizes the aptamer, and the dopamine-bound conformation is more stable than free aptamer conformations, resulting in no observations of aptamer unfolding from the dopamine-bound conformation.

FIG. 9 depicts a representative single-pore recording showing that dopamine fails to bind to the docked aptamer when added to the cis side of the MspA nanopore. Both dopamine (target ligand) and the aptamer (probe) were presented in the cis solution. In equilibrium, the solution contains free aptamer (A₁/A₂), free dopamine and the aptamer⋅dopamine complex (AL). (Left) The first event (marked by red line) has a blocking level consistent with the capture of a dopamine-bound aptamer in the nanopore. Once the dopamine dissociates from the aptamer there are no subsequent dopamine-binding events such as those normally observed when dopamine is added to the trans solution even though dopamine is added at a concentration greatly in excess of the aptamer concentration (25 μM versus 100 nM). This is consistent with the hypothesis that the positive voltage drives the anionic aptamer into the nanopore but prevents cationic dopamine from entering the nanopore (Right). The second aptamer blocking event has a blocking level consistent with the capture of a free aptamer into the nanopore. Therefore, the initial blocking level in the signature gives a ‘snapshot’ for the dopamine-bound conformation captured by the nanopore, but following dopamine dissociation from the aptamer, a subsequent dopamine-binding event is not detected.

FIG. 10A comprises a single-pore current recording showing the aptamer signature and dopamine binding events at 120 mV. Upper trace, 120 mV in the presence of 25 μM dopamine in the trans solution. Since the aptamer residence time (τ_{A}) increases with the voltage many fewer dopamine binding events were observed at lower voltages (marked in black bars). Accordingly, all dopamine binding experiments were conducted at 180 mV.

FIG. 10B comprises a single-pore current recording showing the aptamer signature and dopamine binding events at 150 mV. Lower trace, 150 mV, in the presence of 25 μM dopamine in the trans solution. Since the aptamer residence time (τ_{A}) increases with the voltage many fewer dopamine binding events were observed at lower voltages (marked in black bars). Accordingly, all dopamine binding experiments were conducted at 180 mV.

FIG. 11A shows expanded single-pore recordings showing the binding of non-target ligands serotonin and norepinephrine to the dopamine aptamer from the trans solution for serotonin. Traces were recorded at 100 kHz filtering frequency (upper trace) and further software-filtered at 20 kHz (lower trace), at 180 mV in 1 M KCl (pH7.4) with 100 nM aptamer in cis solution and 50 μM ligands in trans solution.

FIG. 11B shows expanded single-pore recordings showing the binding of non-target ligands serotonin and norepinephrine to the dopamine aptamer from the trans solution for norepinephrine. Traces were recorded at 100 kHz filtering frequency (upper trace) and further software-filtered at 20 kHz (lower trace), at 180 mV in 1 M KCl (pH7.4) with 100 nM aptamer in cis solution and 50 μM ligands in trans solution.

FIG. 12A is a histogram of τ_{off} for binding of non-target serotonin and norepinephrine to the dopamine aptamer and evaluation of missed event percentage for serotonin.

FIG. 12B is a histogram of τ_{off} for binding of non-target serotonin and norepinephrine to the dopamine aptamer and evaluation of missed event percentage for norepinephrine.

FIG. 13A shows single-pore current recordings showing the free serotonin-binding aptamer, the binding of serotonin, and the binding of non-target dopamine to the aptamer from the trans solution for the aptamer in the absence of any ligands. The trace was recorded at 120 mV in 1 M KCl (pH7.4) at a 100 kHz bandwidth (top) and filtered at 20 kHz (middle) and 10 kHz (bottom) by software. The main conformation state A, short intermediates A₁, and ligand-bound aptamer state AL are marked.

FIG. 13B shows single-pore current recordings showing the free serotonin-binding aptamer, the binding of serotonin, and the binding of non-target dopamine to the aptamer from the trans solution for the aptamer in the presence of serotonin. The trace was recorded at 120 mV in 1 M KCl (pH7.4) at a 100 kHz bandwidth (top) and filtered at 20 kHz (middle) and 10 kHz (bottom) by software. The main conformation state A, short intermediates A₁, and ligand-bound aptamer state AL are marked.

FIG. 13C shows single-pore current recordings showing the free serotonin-binding aptamer, the binding of serotonin, and the binding of non-target dopamine to the aptamer from the trans solution for the aptamer in the presence of non-target dopamine. The trace was recorded at 120 mV in 1 M KCl (pH7.4) at a 100 kHz bandwidth (top) and filtered at 20 kHz (middle) and 10 kHz (bottom) by software. The main conformation state A, short intermediates A₁, and ligand-bound aptamer state AL are marked.

FIG. 14 depicts a single-pore current recording showing much shorter residence time of the serotonin aptamer (τ_{A}) and much fewer serotonin binding events at 180 mV, compared with 120 mV As the serotonin aptamer residence time (τ_{A}) was greatly shortened at 180 mV, fewer serotonin binding events are detected (marked in black bars). Therefore, the serotonin aptamer has higher sensitivity at 120 mV This is in contrast to the dopamine aptamer and theophylline aptamer, which have higher sensitivity at 180 mV.

FIG. 15A shows a normalized histogram for the dopamine aptamer residence time in the nanopore τ_{A} in the absence of dopamine and recorded at 180 mV, 150 mV and 120 mV The histogram is plotted with a logarithmic time scale, and τ_{A} values were obtained by fitting a single exponential to the histogram data.

FIG. 15B shows a normalized histogram for the dopamine aptamer residence time in the nanopore τ_{A} in the presence of 25 μM dopamine and recorded at 180 mV and 150 mV. The histogram is plotted with a logarithmic time scale, and τ_{A} values were obtained by fitting a single exponential to the histogram data.

FIG. 16A shows a histogram for obtaining the elapsed time between two consecutive dopamine-binding events (τₒₙ) in different dopamine concentrations, in particular a histogram for τₒₙ in the presence 5 μM dopamine presented in the trans solution. τon was obtained by fitting a single exponential to the histogram data and used to calculate the dopamine binding frequency (f=1/τₒₙ). τₒₙ in 2.5 μM and lower dopamine concentrations was calculated from the arithmetic mean of the collected duration values.

FIG. 16B shows a histogram for obtaining the elapsed time between two consecutive dopamine-binding events (τₒₙ) in different dopamine concentrations, in particular a histogram for τₒₙ in the presence 10 μM dopamine presented in the trans solution. τon was obtained by fitting a single exponential to the histogram data and used to calculate the dopamine binding frequency (f=1/τₒₙ). τₒₙ in 2.5 μM and lower dopamine concentrations was calculated from the arithmetic mean of the collected duration values.

FIG. 16C shows a histogram for obtaining the elapsed time between two consecutive dopamine-binding events (τₒₙ) in different dopamine concentrations, in particular a histogram for τₒₙ in the presence 25 μM dopamine presented in the trans solution. τₒₙ was obtained by fitting a single exponential to the histogram data and used to calculate the dopamine binding frequency (f=1/τₒₙ). τₒₙ in 2.5 μM and lower dopamine concentrations was calculated from the arithmetic mean of the collected duration values.

FIG. 16D shows a histogram for obtaining the elapsed time between two consecutive dopamine-binding events (τₒₙ) in different dopamine concentrations, in particular a histogram for τₒₙ in the presence 50 μM dopamine presented in the trans solution. τₒₙ was obtained by fitting a single exponential to the histogram data and used to calculate the dopamine binding frequency (f=1/τₒₙ). τₒₙ in 2.5 μM and lower dopamine concentrations was calculated from the arithmetic mean of the collected duration values.

FIG. 17A shows a normalized histogram for obtaining the elapsed time for a dopamine-binding event (τₒₙ) at different voltages, namely at 180 mV and 150 mV in 25 μM dopamine.

FIG. 17B shows a normalized histogram for obtaining the elapsed time for a dopamine-binding event duration (τ_{off}) at different voltages, namely τ_{off}, at 180 mV and 150 mV in 25 μM dopamine.

FIG. 18A comprises a histogram for obtaining the serotonin aptamer residence time in the nanopore (τ_{A}). In particular, the figure shows a histogram for τ_{A} in the absence of serotonin at 180 mV. τ_{A} was obtained by fitting a single exponential to the histogram data.

FIG. 18B comprises a histogram for obtaining the serotonin aptamer residence time in the nanopore (τ_{A}). In particular, the figure shows a histogram for τ_{A} in the absence of serotonin at 150 mV. τ_{A} was obtained by fitting a single exponential to the histogram data.

FIG. 18C comprises a histogram for obtaining the serotonin aptamer residence time in the nanopore (τ_{A}). In particular, the figure shows a histogram for τ_{A} in the absence of serotonin at 120 mV. τ_{A} was obtained by fitting a single exponential to the histogram data.

FIG. 18D comprises a histogram for obtaining the serotonin aptamer residence time in the nanopore (τ_{A}). In particular, the figure shows a histogram for τ_{A} in the presence of 25 μM serotonin at 120 mV. τ_{A} was obtained by fitting a single exponential to the histogram data.

FIG. 18E comprises a histogram for obtaining the duration between adjacent serotonin binding events (τₒₙ). In particular, the figure shows a histogram for obtaining τₒₙ in 25 μM serotonin at 120 mV. τₒₙ was obtained by fitting a single exponential to the histogram data.

FIG. 18F comprises a histogram for obtaining the aptamer⋅serotonin binding time (τ_{off}). In particular, the figure shows a histogram for obtaining τ_{off} in 25 μM serotonin at 120 mV τ_{off} was obtained by fitting a single exponential to the histogram data.

FIG. 19A comprises a histogram for obtaining the theophylline aptamer residence time in the nanopore (τ_{A}), in particular a histogram for τ_{A} in the absence of theophylline at 180 mV. τ_{A} was obtained by fitting a single exponential to the histogram data.

FIG. 19B comprises a histogram for obtaining the theophylline aptamer residence time in the nanopore (τ_{A}), in particular a histogram for τ_{A} in the absence of theophylline at 150 mV. τ_{A} was obtained by fitting a single exponential to the histogram data.

FIG. 19C comprises a histogram for obtaining the theophylline aptamer residence time in the nanopore (τ_{A}), in particular a histogram for τ_{A} in the absence of theophylline at 120 mV. τ_{A} was obtained by fitting a single exponential to the histogram data.

FIG. 19D comprises a histogram for obtaining the theophylline aptamer residence time in the nanopore (τ_{A}), in particular a histogram for τ_{A} in the presence of 25 μM theophylline at 180 mV. τ_{A} was obtained by fitting a single exponential to the histogram data.

FIG. 19E comprises a histogram for obtaining the duration between adjacent theophylline binding events (τₒₙ), in particular a histogram for obtaining τₒₙ in 25 μM theophylline at 180 mV. τₒₙ was obtained by fitting a single exponential to the histogram data.

FIG. 19F comprises a histogram for obtaining the aptamer⋅theophylline binding time (τ_{off}), in particular a histogram for obtaining τ_{off} in 25 μM theophylline at 180 mV. τ_{off} was obtained by fitting a single exponential to the histogram data.

FIG. 20 shows an expanded view of block transitions in the serotonin aptamer signature in the presence of serotonin for 40-seconds. The expanded view allows identifying whether the observed transition signals are ligand-free aptamer states between consecutive serotonin-binding blocks (marked by ‘√’) or “noise” flickers of unknown origin (marked by ‘X’). These events have little chance to be missed at the bandwidth of 5 kHz when examined with high time resolution. The trace was recorded at 120 mV, 1 M KCl, 50 μM serotonin in the trans solution.

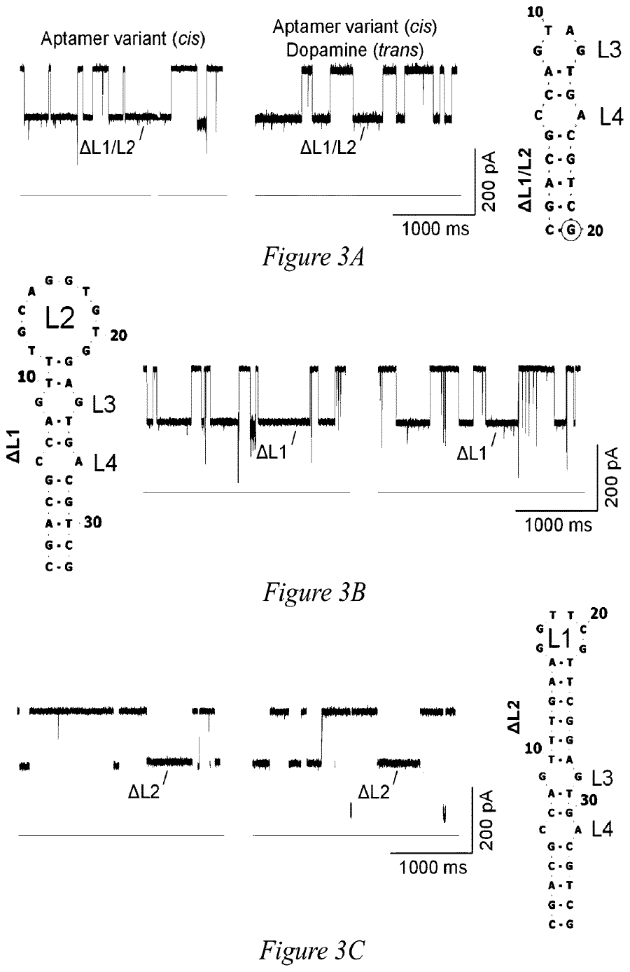

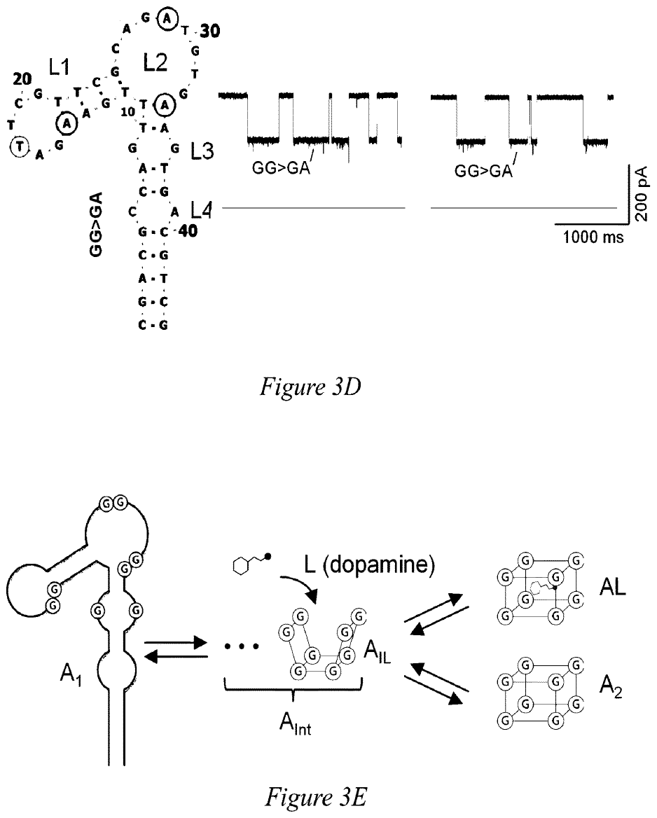

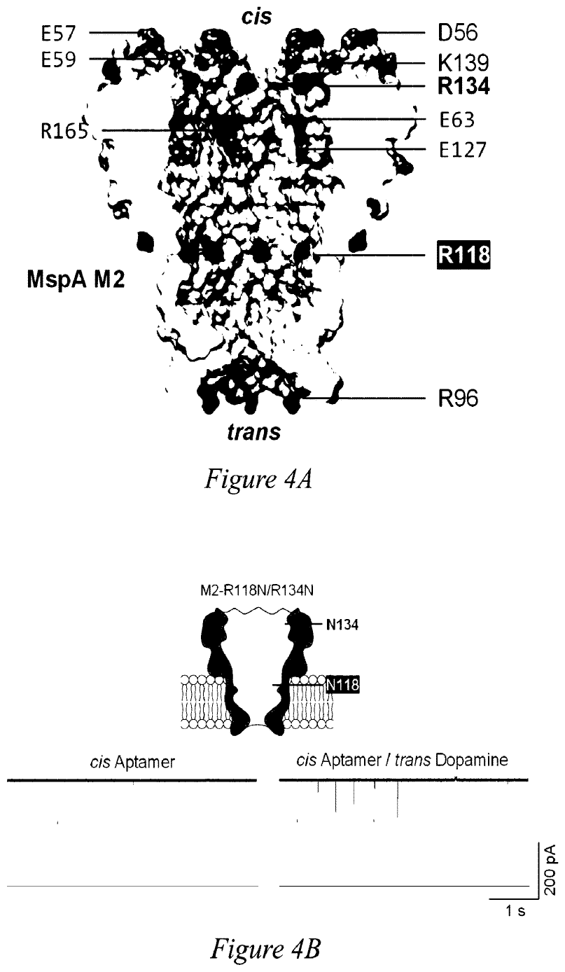

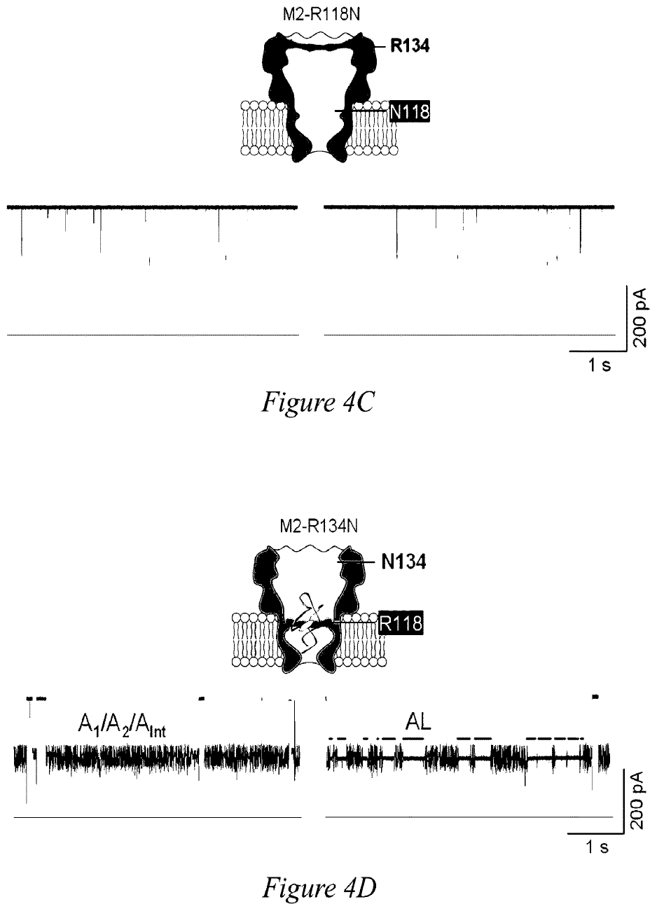

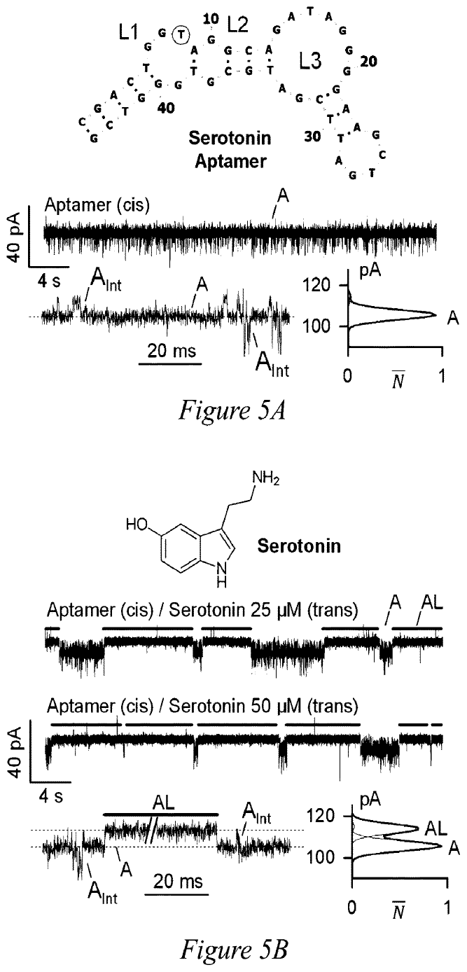

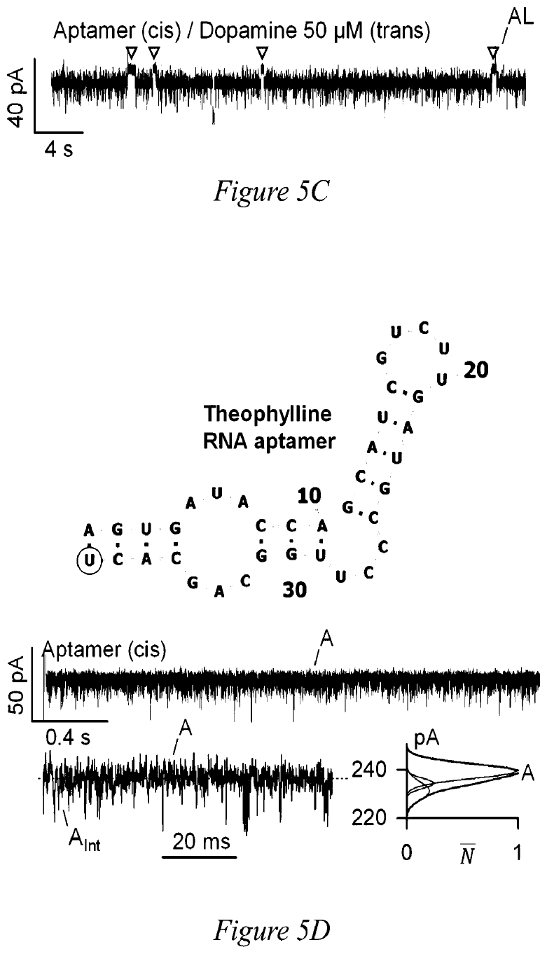

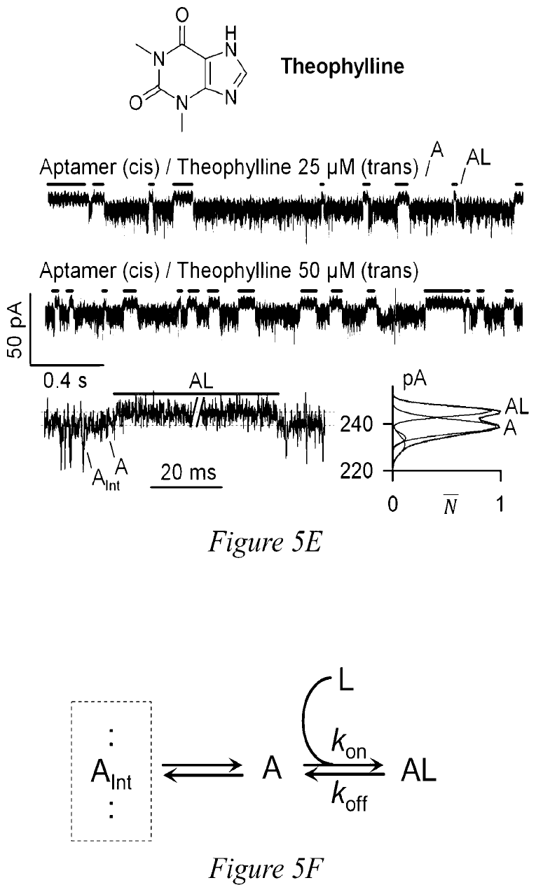

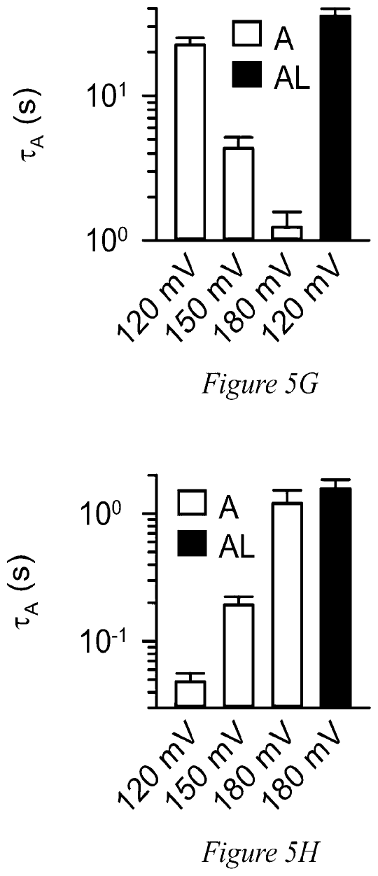

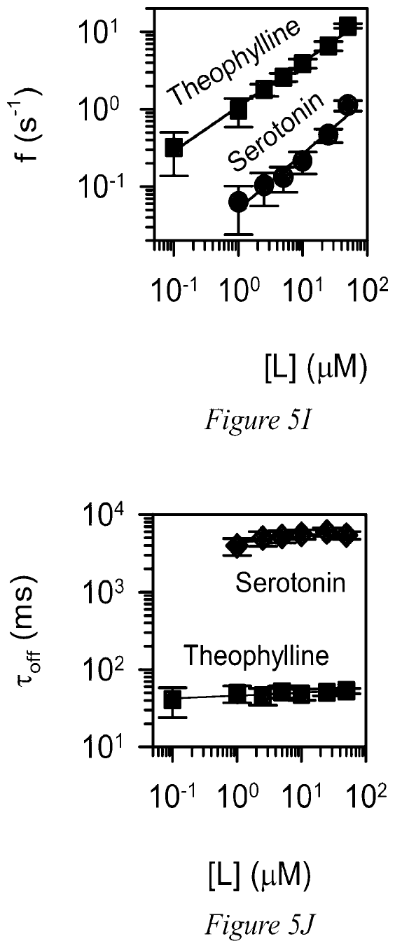

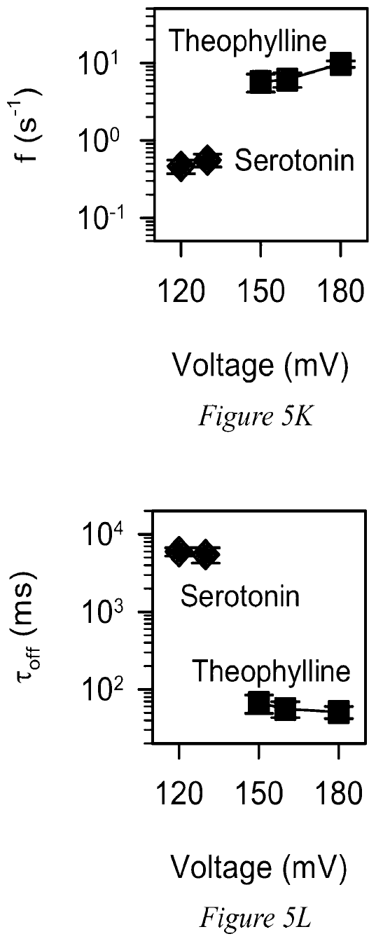

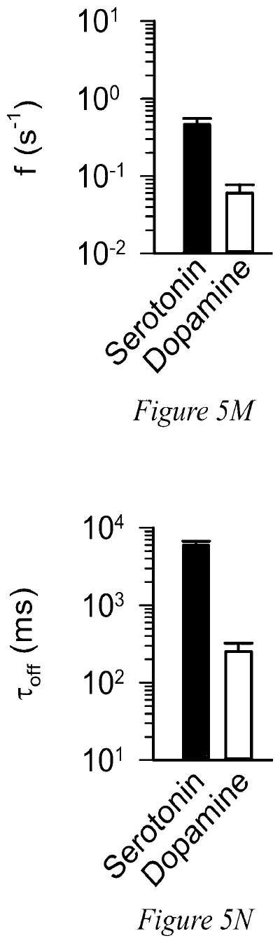

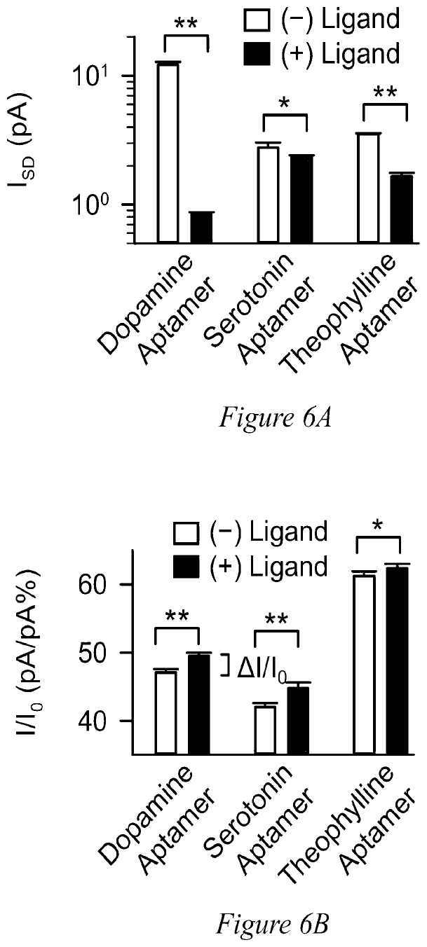

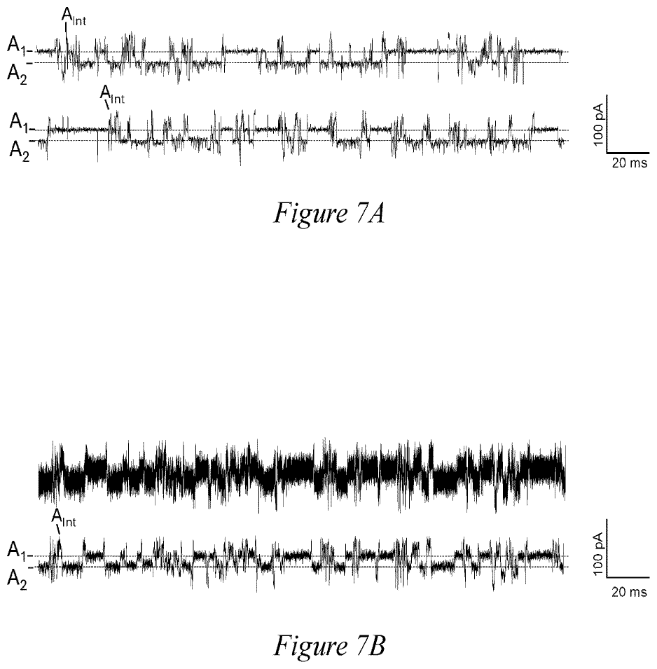

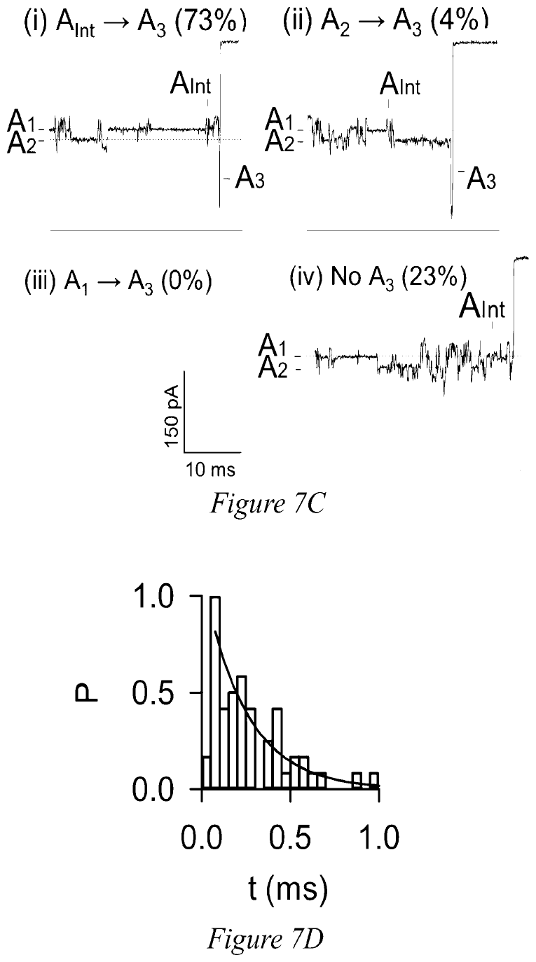

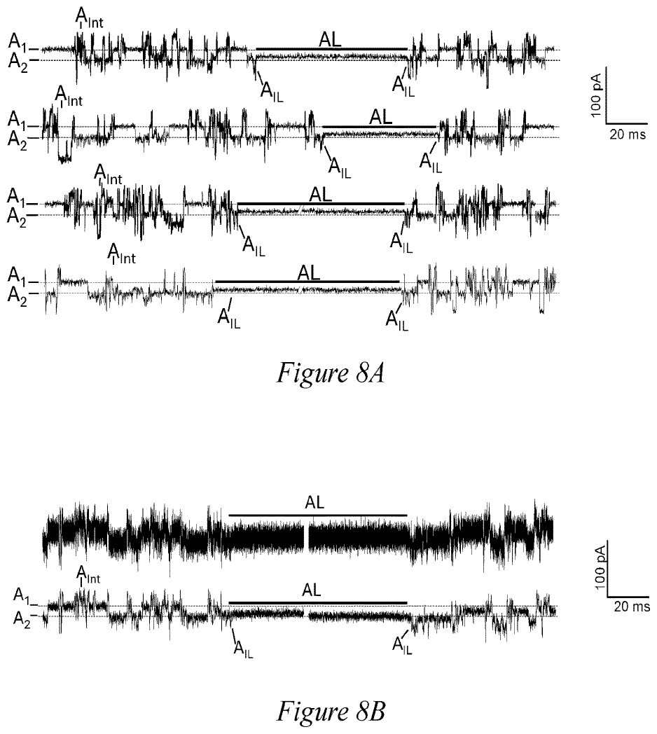

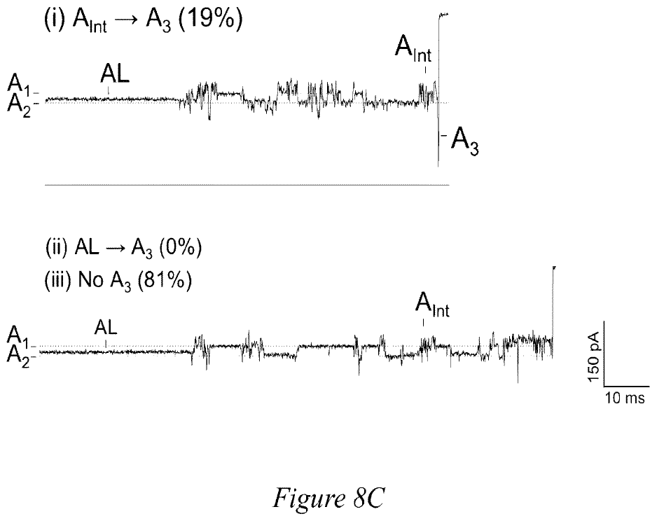

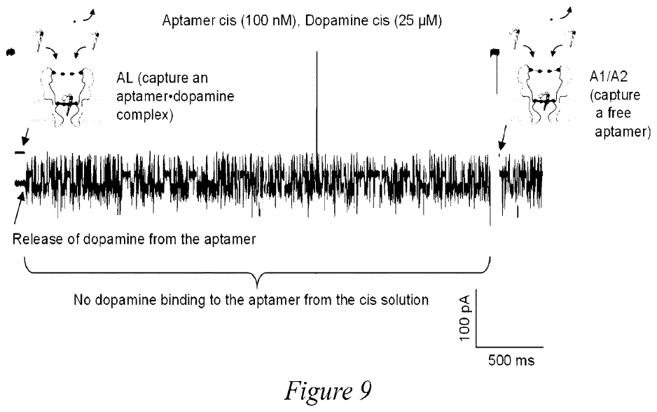

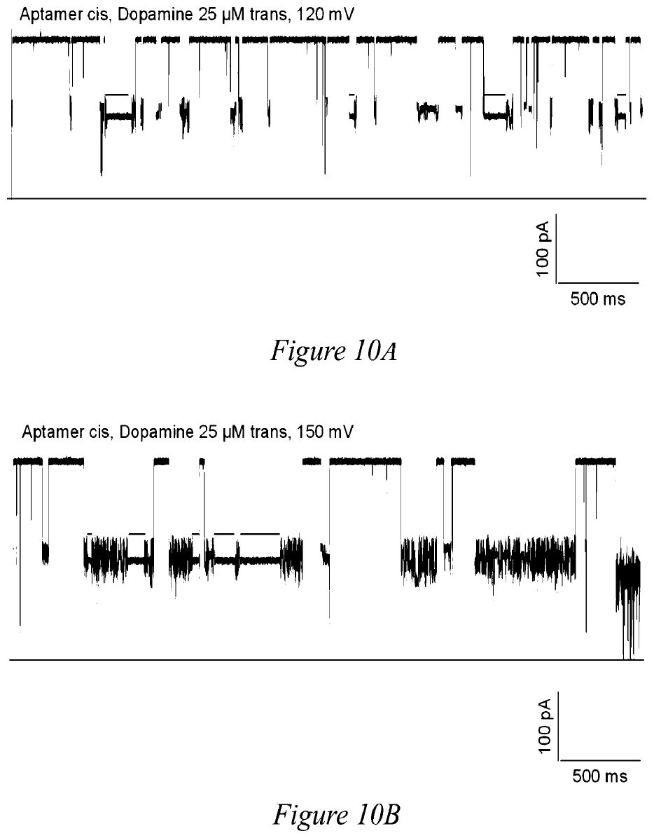

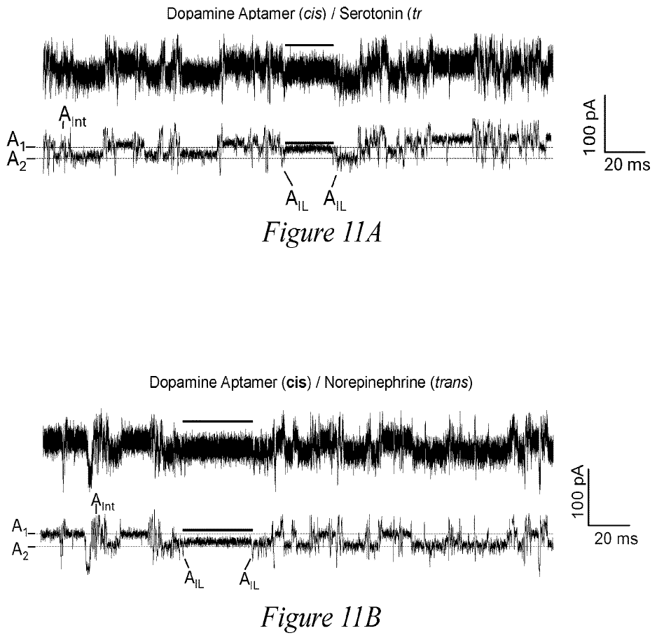

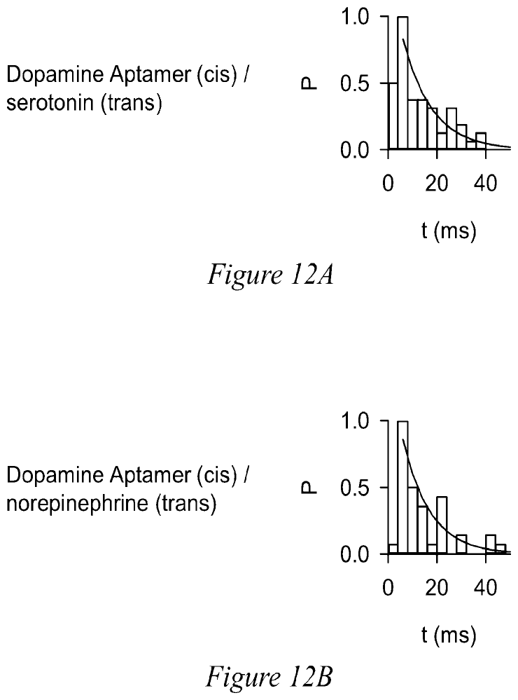

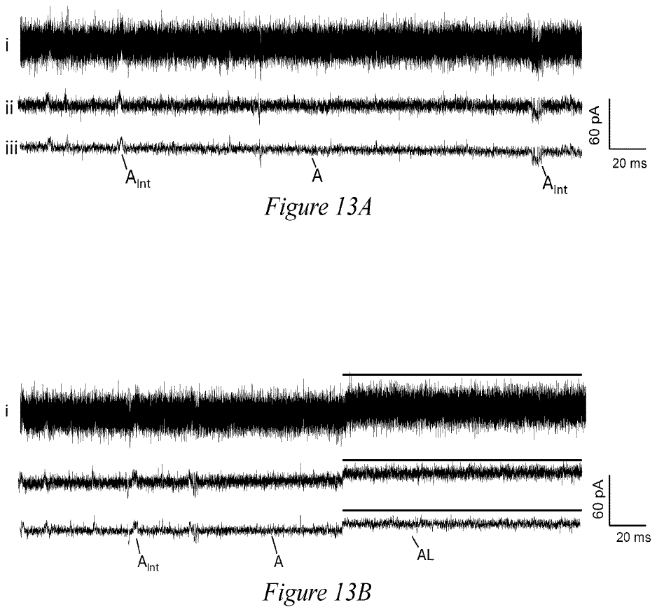

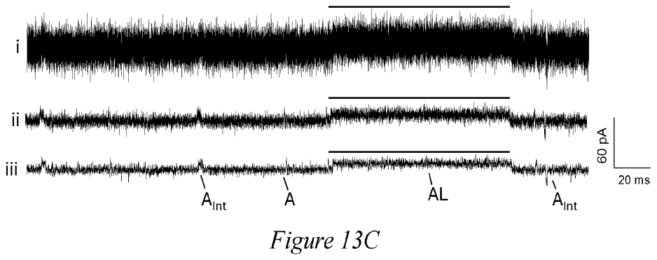

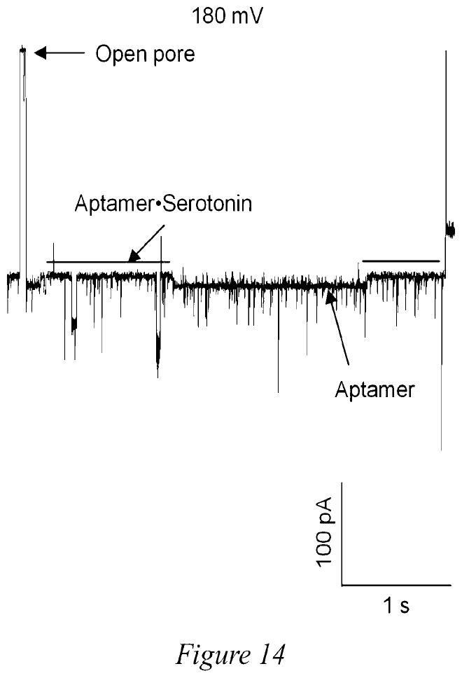

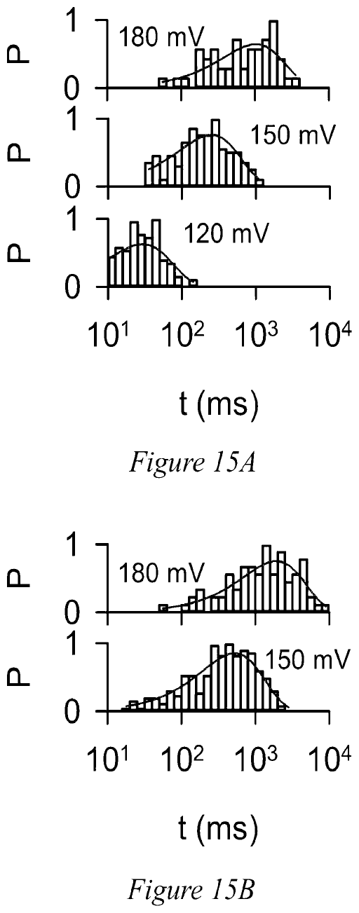

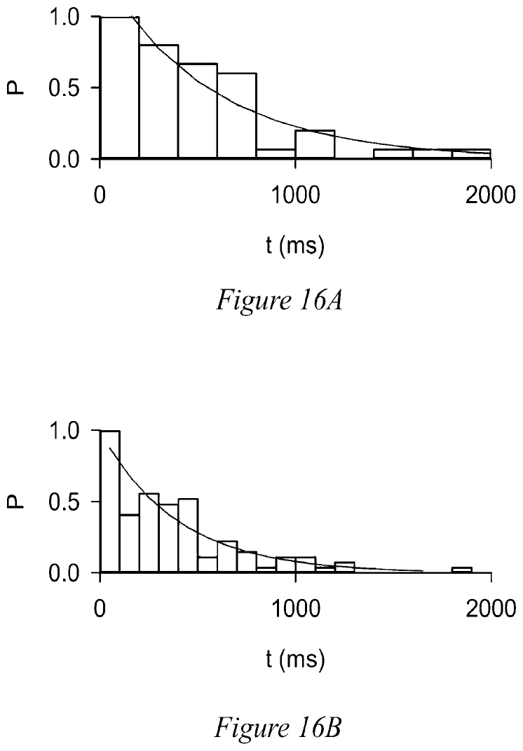

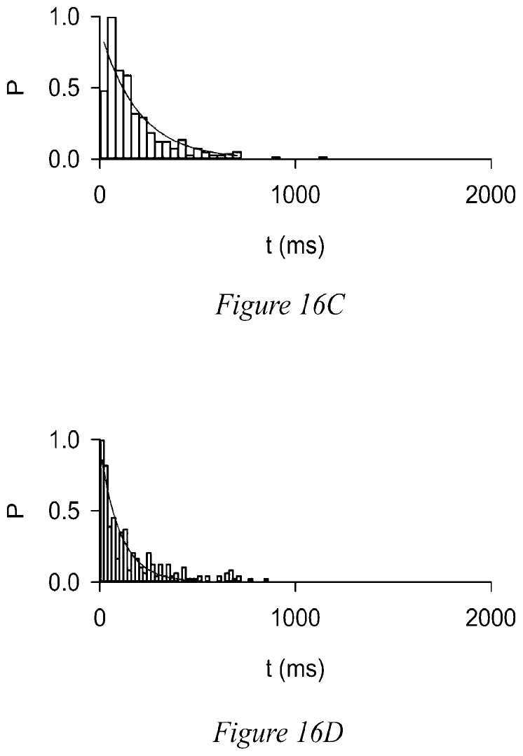

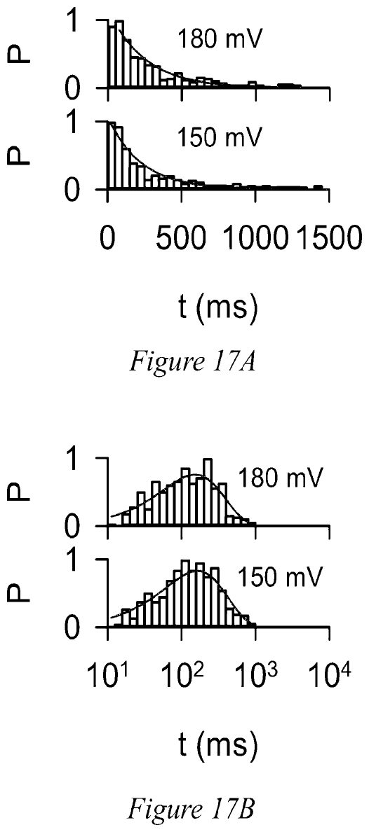

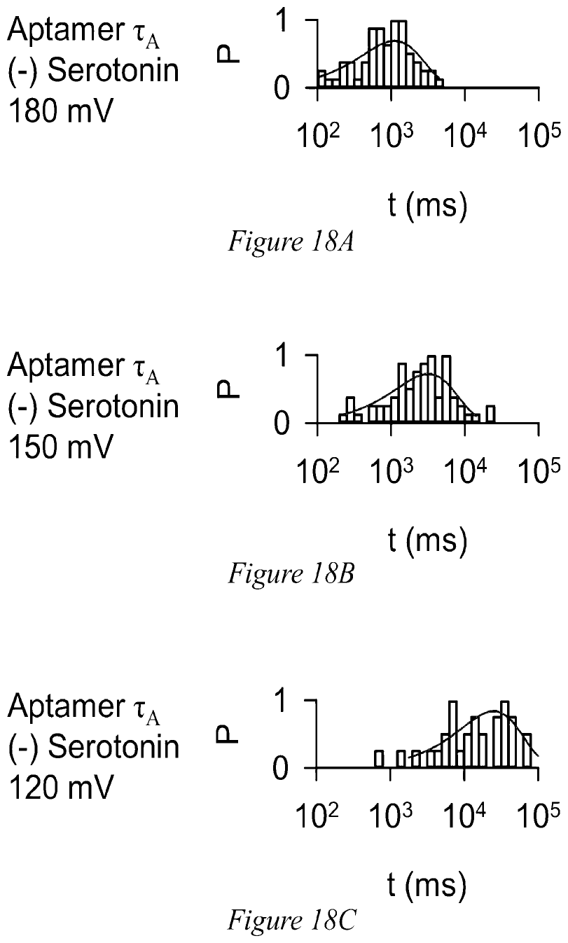

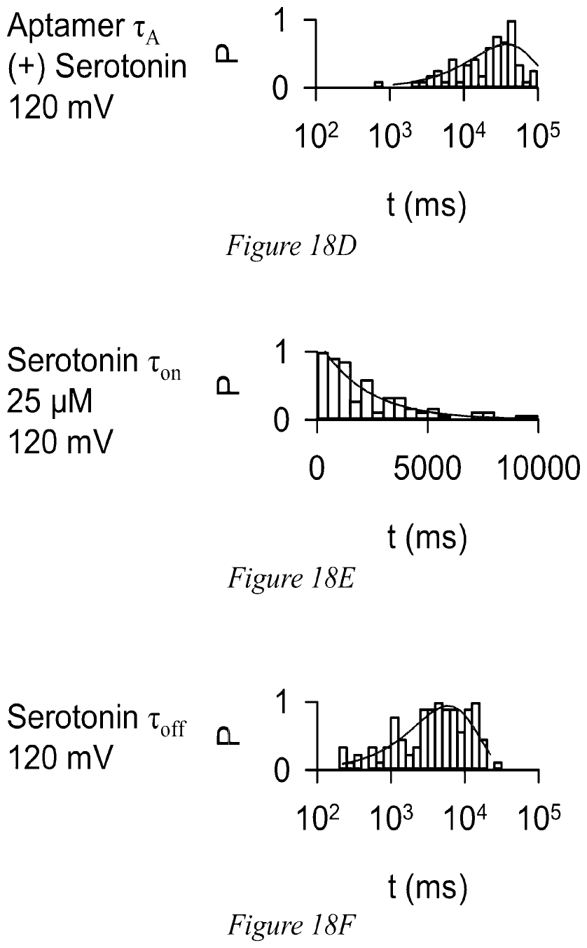

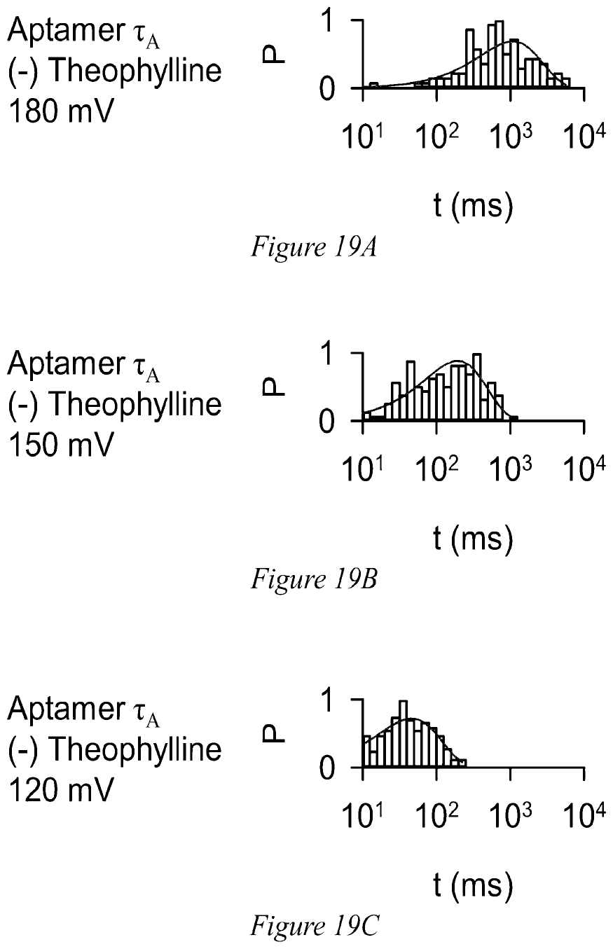

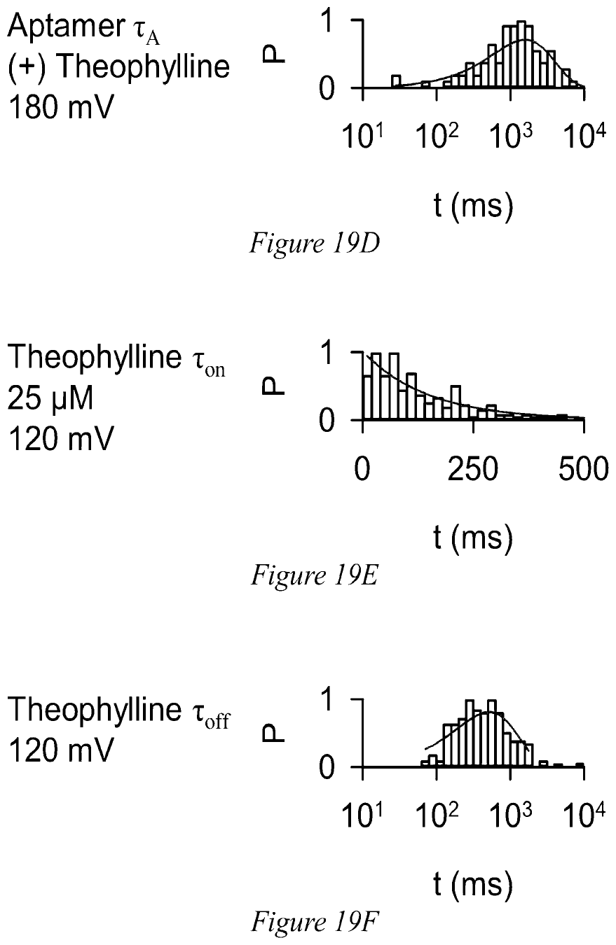

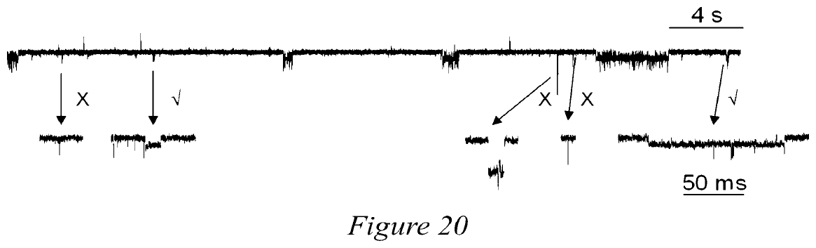

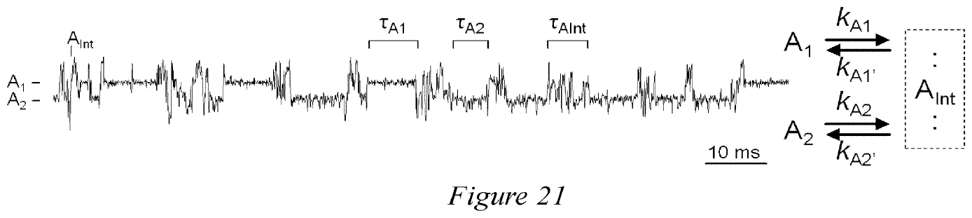

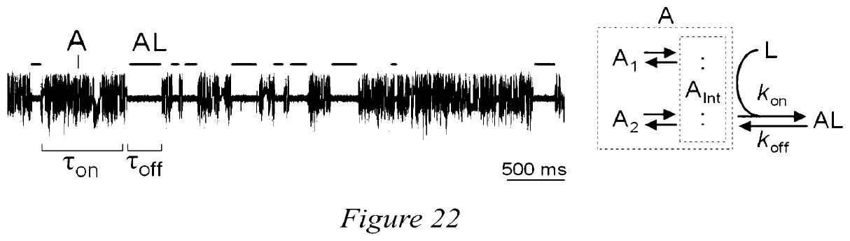

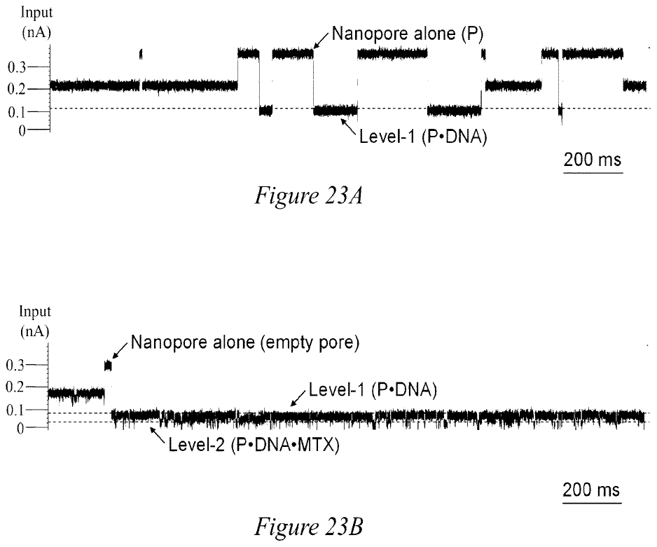

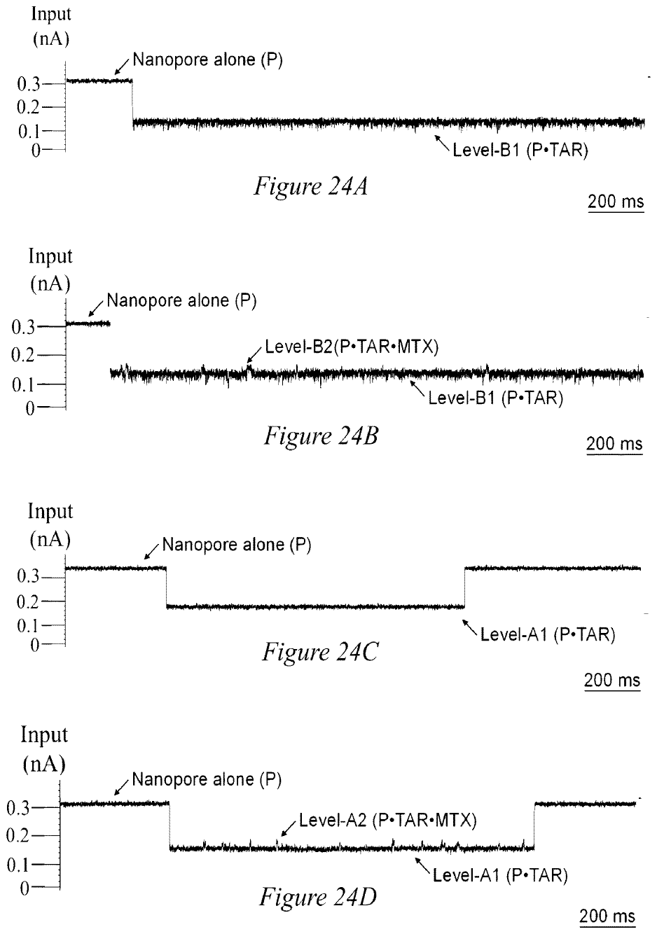

FIG. 21 depicts the kinetics for the dopamine-binding aptamer conformational change in the absence of dopamine.

FIG. 22 depicts the kinetics for the dopamine-binding aptamer conformational change in the presence of dopamine.

FIG. 23A shows the nanopore current signature for docking a 28-bp double-stranded DNA (dsDNA) in the MspA-M2 pore from the cis side. Trace was recorded in 1 M KCl 10 mM Tris (pH7.4) at 150 mV.

FIG. 23B shows the nanopore current signature for binding of Mitoxantrone (MTX) from the trans side to docked DNA in the MspA-M2 pore. MTX concentration was 100 μM. Trace was recorded in 1 M KCl 10 mM Tris (pH7.4) at 150 mV.

FIG. 24A shows the nanopore current signature for docking HIV-1 TAR RNA in the MspA-M7 pore from the cis side. Low-noise event. Trace was recorded in 1 M KCl 10 mM Tris (pH7.4) at 160 mV.

FIG. 24B shows the nanopore current signature for binding of Mitoxantrone (MTX) from the trans side to docked TAR RNA in the MspA-M7 pore. Low-noise event. MTX concentration was 100 μM. Trace was recorded in 1 M KCl 10 mM Tris (pH7.4) at 160 mV.

FIG. 24C shows the nanopore current signature for docking HIV-1 TAR RNA in the MspA-M7 pore from the cis side. High-noise event. Trace was recorded in 1 M KCl 10 mM Tris (pH7.4) at 160 mV.

FIG. 24D shows the nanopore current signature for binding of Mitoxantrone (MTX) from the trans side to docked TAR RNA in the MspA-M7 pore. High-noise event. MTX concentration was 100 μM. Trace was recorded in 1 M KCl 10 mM Tris (pH7.4) at 160 mV.

### DETAILED DESCRIPTION

The present disclosure relates to methods, systems, apparatuses, and compositions for detecting dynamic nucleic acid conformational change in response to small molecule binding. Described herein are modular, label-free, nucleic acid-inlaid nanopores capable of revealing time-resolved, small molecule-induced, single nucleic acid molecule conformational transitions, quantifying specific ligands, elucidating nucleic acid-ligand interactions, and pinpointing the nucleic acid motifs for ligand binding. In particular, as demonstrated by using the dopamine-, serotonin-, and theophylline-binding aptamers as testbeds, these nucleic acids scaffolds can be non-covalently docked inside the MspA protein nanopore by a cluster of site-specific charged residues. This docking mechanism enables the ion current through the nanopore to characteristically vary as the aptamer undergoes conformational changes, resulting in a sequence of current fluctuations that report binding and release of single ligand molecules from the aptamer. The methods, systems, apparatuses, and compositions thereby enable a variety of applications, such as small molecule biosensing, drug discovery, and the design of artificial riboswitch effectors in synthetic biology.

The embodiments of this disclosure are not limited to particular types of compositions or methods, which can vary. It is further to be understood that all terminology used herein is to describe particular embodiments only and is not intended to be limiting in any manner or scope. For example, as used in this specification and the appended claims, the singular forms “a,” “an” and “the” can include plural referents unless the context indicates otherwise. Unless indicated otherwise, “or” can mean any one alone or any combination thereof, e.g., “A, B, or C” means the same as any of A alone, B alone, C alone, “A and B,” “A and C,” “B and C” or “A, B, and C.” Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, a description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6, and decimals and fractions, for example, 1.2, 3.8, 1½, and 4¾ This applies regardless of the breadth of the range.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

The terms “a,” “an,” and “the” include both singular and plural referents.

The term “or” is synonymous with “and/or” and means any one member or combination of members of a particular list, e.g., A, B, and C; A and B; A and C; B and C; or any of A, B, or C individually.

The term “about,” as used herein, refers to variation in the numerical quantity that can occur, for example, through typical measuring techniques and equipment, with respect to any quantifiable variable, including, but not limited to, mass, volume, time, temperature, pH, etc. Further, in practice and implementation, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The methods, systems, apparatuses, and compositions disclosed herein may comprise, consist essentially of, or consist of the components and ingredients described herein as well as other ingredients not described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

The “scope” of the present disclosure is defined by the claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

### Materials, Methods, and Systems for Real-Time Detection of Small Molecule Interactions in a Nanopore

Understanding small-molecule regulation of nucleic acid conformation is important to reveal biological mechanisms and develop biotechnological applications. Many native regulatory nucleic acids such as riboswitches can control gene expression via a conformational transition upon binding with metabolites, serving as new targets for antibiotic design. New therapeutic compounds can be discovered by screening small molecules that bind the target nucleic acid motifs, change their conformation, and modulate their biological functions. Furthermore, in vitro selected nucleic acid aptamers and engineered riboswitches can change their conformation upon ligand binding. Utilizing this property, biosensors can be designed to detect biologically important small molecules, including neurotransmitters and hormones, metabolites, antibiotics, and anticancer drugs, for biological mechanism exploration, disease diagnostics, enzyme profiling and pharmacokinetics studies. In addition, small molecule-sensing aptamers, such as the theophylline aptamer, can be engineered into gene circuits and activated through a ligand-triggered conformational transition to program gene expression and gene editing. To advance the use of small molecule-sensitive nucleic acids as sensors, it is important to develop sensitive, rapid, and low-cost tools that can discriminate different conformations of single nucleic acid molecules and reveal their dynamic transitions in response to small-molecule binding.

Nanopore single-molecule-based biosensing techniques have been applied to sequencing and various genetic, epigenetic and proteomic analyses. By measuring dynamic changes of current through the nanopore, this technique has also demonstrated great potential to detect biomolecular structures. When a protein, DNA, RNA, or nucleic acid/protein complex occludes the nanopore under a transmembrane voltage, their structures can characteristically modulate the ion current through the nanopore. The resulting nanopore current pattern or “signature” can be analyzed to discriminate the molecular structure. However, these nanopore measurements to study biomolecular structure are often limited to providing a conformational “snapshot” and do not reveal the dynamic conformational variation of the detected molecule. This limitation can be overcome by immobilizing the target molecules in the nanopore in a mechanism such that its conformational variation can characteristically modulate the nanopore current. This strategy can be realized by attaching a nucleic acid aptamer or engineering a polypeptide probe to the nanopore to detect reversible binding of a protein ligand outside the nanopore lumen. However, such approaches are generally not sensitive enough to detect small conformational changes of a nucleic acid scaffold upon the binding of a small ligand. In addition, custom fabrication of a nanopore with an attached molecular probe can be complicated. As a noncovalent strategy, a 16-nt compact G-quadruplex in the α-hemolysin nanopore was encapsulated to elucidate its metal ion-regulated folding mechanism, but this nanopore is not wide enough to accommodate most nucleic acid motifs.

Recently, the ClyA protein nanopore has been used to trap a protein molecule in its large cylinder cavity, enabling the nanopore to elucidate protein-protein interactions and detect protein-binding metabolites. There is therefore a need for a new strategy for non-covalent, label-free detection of biomolecular conformational changes confined in a large nanopore. But this wide protein nanopore has not been explored for detecting nucleic acid-small molecule interactions.

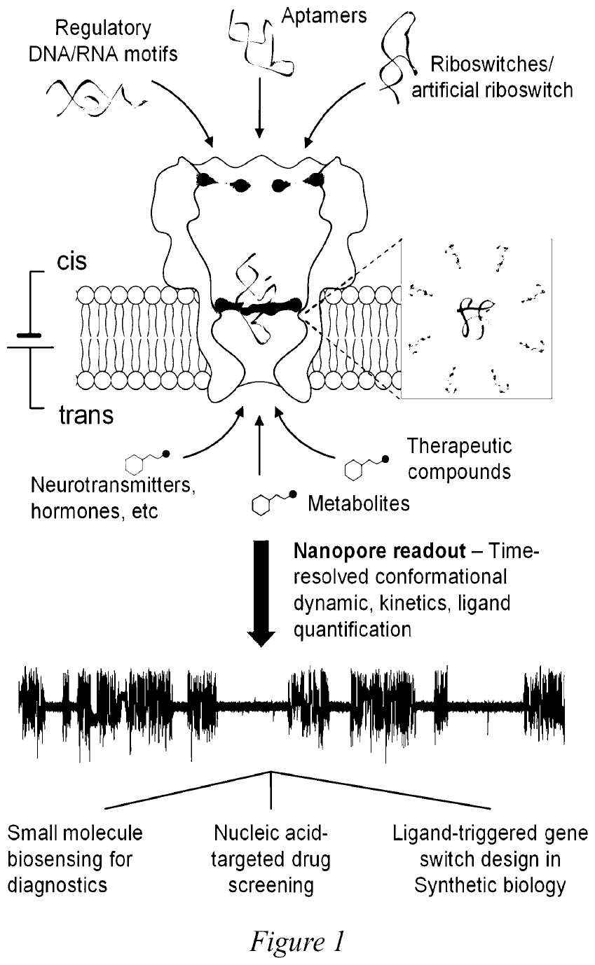

The MspA protein nanopore has been developed for sequencing of nucleic acid and polypeptide, biomolecular mechanistic study, and single-molecule chemistry. This nanopore encloses a 3-5 nm wide goblet-shaped cavity in the cis vestibule that can host nucleic acid scaffolds. Here the dopamine-, serotonin- and theophylline-binding aptamers were used as testbeds to explore a MspA-based, non-covalent, nucleic acid-inlaid nanopore capable of discriminating and continuously recording small molecule-regulated nucleic acid dynamic conformational variation (as shown in FIG. 1) offering a sensor platform for elucidating nucleic acid-small molecule interactions, screening nucleic acid-targeted small molecule regulators, synthetic biology design and small molecule biosensing.

### Dopamine-Induced Aptamer Conformational Transitions in the Nanopore.

MspA forms an octameric protein nanopore in the lipid bilayer. In this study, the mutant M2 nanopore that has been applied in sequencing and biomolecular detection was utilized. This nanopore generated an ion current of I₀=393±7 pA at a transmembrane voltage of 180 mV (conductance of 2.21±0.04 nS). Driven by the voltage, the dopamine-binding aptamer presented in the cis solution produced a long-duration nanopore signature block with a duration of τ_{A}=1.09±0.19 s (see FIG. 2A). The expanded signature shown in FIG. 2A and FIGS. 7A-7D reveals two stable blocking states A₁ and A₂, identified from their distinct blocking levels, I/I₀=53.0±0.3% for A₁ and 47.6±0.4% for A₂.

Most A₁ and A₂ states do not directly transition to each other. Instead, as shown in the greyed intervals of FIGS. 2A-2C, transitions are mediated by a cluster of rapidly transitioning, unstable intermediate states A_{Int}, which have blocking levels that span a wide range above and below A₁ and A₂. As shown in FIGS. 7A-7D, the same current patterns were also identified at a high bandwidth of 100 kHz/400 kHz sampling rate, and no new stable conductance states were revealed in high bandwidth experiments that were not evident at 5 kHz bandwidth. Since discrete conductance levels are not resolvable during the transition interval, each A_{Int} cluster was treated as a single state and describe the aptamer conformation pathway as

A₁←→A_{Int},←→A₂

From the duration of A₁, A₂ and A_{Int}, as well as their translation frequencies, all the transition rates in this kinetic pathway can be derived. It was also determined that many aptamer signatures are terminated with a short-lived deeper block A3 (τ_{A3}=0.39±0.08 ms and I/I₀=16.4±0.9%) (see FIG. 7C). This signal likely represents transient disruption of the aptamer structure followed by translocation to the trans side of the nanopore. In signatures without terminal A₃, our hypothesis is that the aptamer returns to the cis solution before unfolding. In addition, the relative frequencies of A₃ transitions from the other states were characterized (See FIG. 7C). Overall, it was concluded that the dopamine aptamer can be captured by the MspA nanopore from the cis entrance and stably reside in the nanopore while transitioning among multiple conformations.

When dopamine was added in the trans solution and as shown in the intervals of FIG. 2B, a sequence of single-level blocks AL immediately appeared in the aptamer signature. Their blocking level (I/I₀=49.5±0.5%) and duration (τ_{off}=152±18 ms) are both distinct from the free aptamer states. Notably, as shown in FIGS. 2B, 2E, and 2F, their frequency f calculated from the elapsed time between consecutive AL blocks τₒₙ(f=1/τₒₙ), monotonically increased with the dopamine concentration, while their τ_{off} was independent of the dopamine concentration. These properties together suggest that the AL blocks are generated by single dopamine molecules that enter the nanopore from the trans side and bind to the aptamer, resulting in a sequence of dynamic transitions between the free (A) and ligand-bound (AL) aptamer conformations (A←→AL). The kinetics of this pathway can be obtained from the frequency and duration of the AL blocks. Specifically, the association rate constant is kₒₙ=0.24±0.03 μM⁻¹·s⁻¹, calculated from f=kₒₙ[L]([L]=25 μM dopamine); the dissociation rate constant is k_{off}=6.6±0.8 s⁻¹, calculated from k_{off}=1/τ_{off}; and the dissociation constant is k_{off}=27±3 μM, calculated from k_{off}/kₒₙ. It was found that addition of dopamine on the trans side is a prerequisite for generating sequential dopamine binding events. As shown in FIG. 9, dopamine added in the cis solution where the aptamer is presented cannot produce the AL blocks, presumably because the positive voltage applied to promote entry of the anionic aptamer into the nanopore repels cationic dopamine from entering from the cis side.

It was found that the optimal voltage to detect sequential dopamine binding events is ~180 mV As shown in FIG. 2D, the aptamer residence time τ_{A} increases with the voltage, but saturates for voltages near 180 mV, which is also near the limit to maintain a stable lipid bilayer. As shown in FIGS. 10A-10B, aptamers with long τ_{A} can capture many dopamine molecules, whereas that with too short τ_{A} at low voltages such as 120 mV cannot capture dopamine. Over the range of 150-180 mV, and as shown in FIG. 2G and FIG. 2H, it was found that the frequency (f) and duration (τ_{off}) of the AL blocks are weakly voltage-dependent, a phenomenon probably resulting from the uneven field distribution in the nanopore. Presumably, a large fraction of the voltage falls on the short restrictive sensing zone at the trans end of the nanopore, whereas a small fraction of the voltage forms a weak field across the large cavity where dopamine binds to the aptamer, reducing the voltage impact on the aptamer-ligand interactions.

The aptamer signatures were analyzed to elucidate the native properties of the aptamer-ligand interactions. First, the expanded signatures shown in FIG. 2B and FIGS. 8A-8C demonstrate that the AL block was generated from transition levels A_{Int} with greater block than A₂, and upon dopamine dissociation, returns to a similar A_{Int} state before resuming the free aptamer kinetics. This finding suggests that, among multiple conformations adopted by the free aptamer, dopamine selectively binds to the aptamer occupying an unstable (A_{Int}), rather than one of the two stable states (A₁, A₂), demonstrating a conformation-selectivity for ligand binding. Second, as seen in FIG. 2D, the aptamer's residence time τ_{A} was almost doubled from 1.02±0.19 s in the absence of dopamine to 1.90±0.29 s in the presence of 25 μM. Also, as shown in FIG. 8C, the aptamer residing in the nanopore does not unfold at the AL state (no terminal A₃ block). Both findings demonstrate the stabilization effect of ligand binding on the aptamer structure. Third, chemically related neurotransmitters on the trans side were screened, and found that both serotonin and norepinephrine can also bind the dopamine aptamer (see FIG. 2c, FIGS. 11A-11B, and FIGS. 12A-12B).

In particular, as shown in FIGS. 12A-12B, the ligand binding events (AL) were collected from the recordings at 180 mV in the presence of 50 μM ligand on the trans side. τ_{off} was 16±4 ms for serotonin and 18±5 ms for norepinephrine. Single-pore signatures clearly show that the durations for serotonin and norepinephrine binding to the dopamine aptamer are 9-fold and 8-fold shorter than dopamine (157 ms). The limited temporal resolution can result in missing short binding events and skew the analysis of the on rate. The number of missed events can be estimated from the exponential lifetime distribution. The fraction of missed events is given by 1−exp(−tₘᵢₙ/τ), where tₘᵢₙ is the minimum resolvable dwell time. For a 5 kHz bandwidth, the 10-90% rise time is ~60 us, but bandwidth is not the major determinant of tₘᵢₙ. Rather, the fluctuating nature of the A₁ states leads us to estimate our tₘᵢₙ to be ~2 ms. In this case, with a mean dwell time (time constant) of ~20 ms for serotonin and norepinephrine, it was estimated that 10% of binding events will be missed. This will somewhat skew the estimate of the on rate but will not introduce fundamental errors. In addition to the 5 kHz bandwidth recordings, expanded signatures were demonstrated at 100 kHz bandwidth without filtering and filtered at 20 kHz for the dopamine aptamer in the presence of serotonin or norepinephrine (see FIGS. 11A-11B).

However, as seen in FIG. 2J, the binding time, τ_{off}=16±4 ms for serotonin and 18±4 ms for norepinephrine, were distinctly shorter than for dopamine (τ_{off}=57 ms), suggesting an approximately 10-fold faster dissociation process for the two non-target ligands. Meanwhile, as shown in FIG. 21, both neurotransmitters bind to the aptamer at much lower frequencies, with f=0.27 s⁻¹ for serotonin and 0.20 s⁻¹ for norepinephrine (50 μM), compared with dopamine (f=6.1 s⁻¹, resulting in an approximately 25-fold slower association process. With these kinetic parameters it was estimated that about 10% of the binding events will be shorter than 2 ms and therefore escape detection as indistinguishable from the free aptamer intermediates (see FIGS. 12A-12B). Therefore, this will not introduce fundamental errors to the association rate estimation. It was concluded that the aptamer is dopamine-selective for both the association and dissociation processes, which together result in a 230- and 260-fold decrease in the affinity (increase in K_{d}) for serotonin and norepinephrine.

### Identifying Dopamine Binding Motifs by Screening Aptamer Variant-Ligand Interactions

The secondary structure of the dopamine aptamer consists of a hairpin loop L1 and a multi-branch loop L2 (see FIG. 2A). In addition, the two single nucleotide internal loops L3 and L4 function as a joint, allowing the rigid main helix to bend and twist in the tertiary structure. How the aptamer forms a tertiary structure for dopamine binging and the key motifs participating in dopamine binding are not known. As shown in FIGS. 3A-3E, the aptamer-inlaid nanopore was used to screen a group of aptamer variants. Through comparison of their conformations and their interactions with the ligand, the key motifs on the aptamer structure can be identified for dopamine binding.

The functions of loops L1 and L2 were investigated. The variant ΔL1/L2 deletes both loops L1 and L2 (see FIG. 3A), and L1 and L2 only deletes L1 (see FIG. 3B) or L2 (see FIG. 3C) respectively. Unlike the original aptamer that produced multi-level current blocks in the absence of dopamine as seen in FIG. 2A, all the three variants produced long-duration single-level blocking events, with I/I₀=54.2±1.2% and τ_{A}=34±5 ms for ΔL1/L2, I/I₀=56.9±1.1% and τ_{A}=46.3±1.6 ms for ΔL1, and/I₀=53.1±0.8% and τ_{A}=71.3±5.9 ms for ΔL2 (see FIGS. 3A-3C, left traces). Addition of trans dopamine did not generate any AL binding events (see FIGS. 3A-3C, right traces). The loop screening indicates that unlike the original aptamer that rapidly transitions between multiple conformations, the variants with deletion of both loops L1 and L2 (ΔL1/L2) or either L1 (ΔL1) or L2 (ΔL2) only adopts a single resolvable current-blocking conformation that loses the sensitivity to dopamine, presumably because dopamine binding requires both loops L1 and L2. Without the tertiary folding capability, these loop variants should maintain a hairpin conformation, generating single-level blocks in a characteristic current range (I/I₀=53-57%).

It has been speculated that the binding of dopamine may induce the aptamer to form a G-quadruplex. If so, it was anticipated that this should be a compact G-quadruplex consisting of two quartets contributed by four GG motifs: G16/G17 in L1, G28/G29 and G33/G34 in L2, and G9/G36 in L3, and the G-quadruplex formation can be disabled in the partial absence of these guanine bases. The GG motifs in the loops were targeted by testing the variant GG>GA that carries the G>A single nucleotide polymorphisms (SNPs) at G17, G29 and G34 (see FIG. 3D).

This variant produced blocking patterns very similar to those produced by the loop-deletion variants with a single blocking level of I/I₀=57.3±0.8% and duration of τ_{A}=76±5 ms (see FIG. 3D, left trace). The presence of trans dopamine did not produce any apparent dopamine binding events, and the signature's blocking level and duration were not significantly changed, with I/I₀=56.7±0.7% and τ_{A}=44±5 ms (see FIG. 3D, right trace). Therefore, without the three guanine bases distributed in L1 and L2 that enable the G-quadruplex formation, the aptamer can only adopt a single resolvable dopamine-insensitive blocking conformation. Its blocking level (57%) is similar to that of the three loop deletion variants (I/I₀=53-57%), in consistence with that this SNP variant could form a hairpin. In summary, the selected guanines in the loops are among the key motifs to the formation of the G-quadruplex upon dopamine binding.

The aptamer and its variant studies suggest a conformation model for the aptamer-dopamine interaction (see FIG. 3E). The A₁ state of the free aptamer can be assigned to a hairpin, as its blocking level (I/I₀=53.0%) is similar to that of the four hairpin variants (I/I₀=53-57%), including the point-mutation variant (GG>GA) without changing the aptamer length. A₂ of the free aptamer could adopt a short-lived G-quadruplex that is folded from the hairpin (A₁) via unstable intermediates A_{Int}. The intermediates with low blocking levels can form a docking site accessible to dopamine. Dopamine is selectively docked in these intermediates, inducing the aptamer to form a stable G-quadruplex (AL).

Docking a Dopamine Aptamer with a Cationic Ring Engineered in the MspA Nanopore

The ability to continuously observe aptamer dynamic conformational transitions originates from the aptamer docking configuration in the nanopore, which should not only stably hold the aptamer in the nanopore, but also enables the nanopore current to be sensitively modulated by the aptamer conformation. Here a group of mutant nanopores was used to explore where and how the aptamer is docked in the nanopore. Supposing that positively charged amino acids in the lumen participate in the nanopore-aptamer interaction, mutations were selected which altered the nanopore charge distribution. As shown in FIG. 4A, the candidate aptamer locations in the M2 nanopore include the R118 ring in the middle of the nanopore and the R134 ring near the cis entrance. R165 in between the two rings was not considered because its side chain does not project to the lumen and its charge may be balanced by surrounding E63 and E127.

The mutant M2-R118N/R134N nanopore, which replaces both R118 and R134 rings with neutral asparagine (see FIG. 4B, model), was first tested. It was found that the cis aptamer no longer produced the M2-like prolonged block at 180 mV (see FIG. 4B, left trace), but only short-lived partial blocks (I/I₀=61.2±0.9%, τ_{A}=1.4±0.4 ms), indicating that the nanopore without both positively charge rings cannot effectively capture the aptamer. The mutant M2-R118N nanopore, which removes the R118 ring but retains the R134 ring (see FIG. 4C) was further tested. Again, only brief blocking events were observed (see FIG. 4C, left trace, I/I₀=60.7±0.8%, τ_{A}=0.7±0.3 ms). As neither nanopore can effectively capture the aptamer, it is unsurprising that there were no dopamine binding events from the trans solution (see FIGS. 4B-4C, right traces). The mutant M2-R134N nanopore, which retains the R118 ring but removes the R134 ring, was also tested (see FIG. 4D, model). Interestingly, this nanopore recapitulates the functional interactions with the aptamer found in the native M2 nanopore. The aptamer generated the M2-like blocking signature currents (see FIG. 4D, left), which feature a long residence time (τ_{A}=1.5±0.2 s) and transitions between two main conformations, A₁ at I/I₀=50.7±0.6% and A₂ at I/I₀=47.9±1.0%, via a cluster of unstable transitions. Dopamine can also bind the aptamer in the nanopore from the trans side (see FIG. 4D, right trace, marked by black lines), with similar kinetics (kₒₙ=0.14±0.03 μM⁻¹·s⁻¹ and k_{off}=4.9±0.7 s⁻¹) as wild type.

In conclusion, by screening the charge distribution in the nanopore lumen, it was identified that the R118 ring, rather than the R134 ring, plays a key role in docking the aptamer. This finding supports that the aptamer is very likely suspended in the middle of the lumen cavity, where it is coordinated by the positive charges on the R118 ring. This docking configuration has several functions: (i) the “suspended” aptamer is fully exposed to the surrounding ion pathway, enabling the ion current to sensitively change with the aptamer conformation; (ii) the aptamer does not block the ligand pathway at the narrow trans entrance, allowing the ligand to flow through the nanopore from the trans side to interact with the “suspended” aptamer from different directions, regardless the orientation of the aptamer's ligand binding site; and (iii) The multiple blocking levels in the aptamer signature (e.g., A₁ and A₂) are confirmed to be generated by different conformations, rather than different locations of the aptamer in the nanopore.

### a Platform for Assaying Nucleic Acid-Small Molecule Interactions

A serotonin-binding aptamer and a theophylline riboswitch RNA aptamer were also investigated to demonstrate broader applications of this nanopore platform for detecting small molecule-induced nucleic acid conformation changes. The study of the serotonin aptamer further enhances its potential for neurotransmitter detection, and the study of the theophylline aptamer expands the target to small molecule-sensitive RNA scaffolds.

Similar to the dopamine aptamer and as shown in FIG. 5A, the serotonin aptamer can be stably docked in the M2 nanopore from the cis side at 120 mV, producing a long signature at a blocking level of I/I₀=42.0±0.6% (120 mV). The binding of serotonin from the trans side characteristically increased the blocking level to I/I₀=44.8±0.8%, generating a sequence of AL blocks with τ_{off}=5.9±0.7 s for the ligand-bound aptamer conformation (see FIG. 5B). As the serotonin concentration increased, the frequency f of the AL blocks increased (see FIGS. 5B and 5I) and their duration τ_{off} was not changed (see FIG. 5J). Both f and τ_{off} are not significantly influenced by the voltage applied (see FIGS. 5K and 5L). Serotonin binding can stabilize the aptamer, increasing its residence time τ_{A} by 1.6 folds from 22.4±2.7 s in the absence of serotonin to 35.5±4.5 s in 25 μM serotonin (see FIG. 5G).

As seen in FIG. 5C, dopamine rarely binds to the serotonin aptamer. As depicted in FIGS. 5M and 5N,f for dopamine binding was 7-fold lower, and τ_{off} was 23-fold faster than serotonin binding, respectively, suggesting that the aptamer is serotonin-selective.

The serotonin and dopamine aptamers also show significant differences in blocking conformations and kinetics: (i) In contrast to the dopamine aptamer that transitions between two resolvable conformations (A₁ and A₂), the serotonin aptamer folds into one resolvable conformation A (see FIG. 5A. In addition, as shown in FIG. 5A and FIGS. 13A-13C, the aptamer also generates several types of sub-millisecond to millisecond intermediates with small probabilities. (ii) Serotonin directly binds to the main conformation A (see FIG. 5A and FIGS. 13A-13C), in contrast to dopamine that binds to an intermediate state. (iii) From f (see FIG. 5K) and τ_{off} (see FIG. δI, the kinetic constants were calculated to be kₒₙ=0.0185±0.0037 μM⁻¹·s⁻¹ k_{off}=0.167±0.022 s⁻¹, and k_{d}=9.0±1.3 μM. The kinetic analysis suggests that the binding of serotonin to its aptamer is 9-fold slower in the association process and 40-fold slower in the dissociation process compared to dopamine binding to its aptamer. (iv) In contrast to the dopamine aptamer that reaches the highest stability and sensitivity at 180 mV, the serotonin aptamer is more stable in the nanopore at lower voltages (see FIG. 5G), and therefore can generate more serotonin binding events at 120 mV than at 180 mV (see FIG. 14).

Similar to the dopamine aptamer, the theophylline aptamer can be docked in the nanopore from the cis solution (see FIG. 5D) with τ_{A} that is prolonged to 1.4±0.3 s as the voltage increases to 180 mV (see FIG. 5H). Similar to the serotonin aptamer, this aptamer's signature is a single-level current block at I/I₀=61.2±0.9% with 100-μs-scale, low-probability blocking flickers, indicating that the aptamer without theophylline binding adopts a single resolvable main conformation (A) that transitions with short-lived intermediates at lower blocking level than A. Theophylline can bind the docked aptamer from the trans solution, generating consecutive single-level blocking events with τ_{off}=51±9 ms (see FIG. 5E), which slightly increase the current level from I/I₀=61.2±0.9% for free aptamer conformation (A) to I/I₀=62.4±0.7% for the theophylline-bound state (AL), with its f increasing with the theophylline concentration (see FIG. 5I). Similar to both the dopamine and serotonin aptamers, f and τ_{off} for theophylline binding to its aptamer are weakly voltage dependent (see FIGS. 5K and 5I). From f and τ_{off} (180 mV, 25 μM theophylline), it was calculated that τₒₙ=0.269±0.043 μM⁻¹·s⁻¹, τ_{off}=19.6±2.7 s⁻¹, and K_{d}=72±17 μM. In addition, the theophylline binding can also stabilize its aptamer, increasing τ_{A} by 29% from 1.21±0.32 s without ligand to 1.58±0.27 s with ligand. Overall, it was verified that this nanopore can detect conformational changes of an RNA scaffold interacting with its small molecule ligand.

Two common conformational properties of the three aptamer-ligand interactions revealed by the characteristic nanopore blocking levels can be summarized as follows. One common feature is that the aptamer alone transitions among multiple conformations, whereas ligand binding transforms the aptamer into a single stable ligand-bound conformation. This property markedly reduces the nanopore current fluctuations (I_{SD}, standard deviation of the nanopore current) upon ligand binding to the aptamer (see FIG. 6A). For the dopamine aptamer, the reduction in current fluctuations is primarily due to the stabilization of the aptamer such that it no longer fluctuates between the A₁ and A₂ conformations, which block the current to different extents. For the serotonin and theophylline aptamers, fluctuations are also reduced upon their ligands binding to their respective aptamers, even though only a single current-blocking conformation is resolved before ligand binding. The reduction in current fluctuation is presumably because the aptamer assumes a more stable configuration in the nanopore upon ligand binding. Therefore, detection of a ligand-binding event is possible by observing changes in current fluctuations for all the aptamers.

A second notable common feature is that the three aptamers' ligand-bound conformations block the current to a lesser extent compared to the ligand-free conformation (see FIG. 6B). For the dopamine aptamer, dopamine binds to an A_{IL} intermediate to form a ligand-bound conformation AL. AL can be distinguished from the A_{IL} as its current level is higher than A_{IL} by I/I₀=4.3% (~19 pA). The serotonin and theophylline aptamers follow the same trend: the current level for the ligand-bound conformation (AL) is higher than the free aptamer (A) by IΔ/I₀=2.8% (~7 pA) for the serotonin aptamer and ΔI/I₀=1.2% (~5 pA) for the theophylline aptamer. This common property provides insight into the aptamer structure variation upon ligand binding. It was therefore determined that ligand binding causes the aptamer to form a more compact structure that slightly decrease in volume, due to the coordination between the ligand and docking site. The smaller molecular volume decreases the occlusion of the ion pathway, thereby increasing the rate of ion flow through the nanopore.

A rapid, sensitive and label-free platform was developed to study dynamic nucleic acid-small molecule interactions at high temporal resolution. The ligand screening experiment for aptamer selectivity demonstrates the potential of the platform in screening for small molecule regulators or potential therapeutic agents targeting nucleic acid motifs. Small molecules are regulators of various nucleic acid structures and functions, such as riboswitch aptamers, HIV TAR⁸⁹, HCV internal ribosome entry site, SERS-CoV-2 frameshifting element, human microRNA and RNA repeats. The aptamer motif screening experiment for ligand binding demonstrates the potential of the platform in gene switch design for programming cellular functions by in vitro screening for their conformation changes resulting from binding triggering ligands. Although the three ligands in this study are cationic compounds, anionic ligands could also be tested. Theoretically, anionic ligands can be presented in the cis solution, to be electrically dragged into the nanopore from the cis entrance by a positive voltage to bind the aptamer residing in the nanopore.

For small molecules sensing, neurotransmitters are commonly detected by measuring the electron transfer between the transmitter and the electrode. However, this electrochemical approach is restricted to oxidizable neurotransmitters, and is complicated by signal interference in the presence of multiple oxidizable compounds and impurities. Aptamers can target both oxidizable and non-oxidizable neurotransmitters, and its ligand selectivity diminishes non-target interference. Neurotransmitters can also be detected by binding with a labeled reporter, which is mixed with the target in the solution to reveal its spatial and temporal distribution. However, the mixture needs time to get to equilibrium, causing limited applications in fast detection of neurotransmitter. In the nanopore method, the aptamer is separated from the ligand by the membrane. They do not mix in the same solution and are instead driven by the imposed electric field to interact within the nanopore. This configuration allows detecting the ligand immediately when it binds to the aptamer without the need to come to equilibrium with the aptamer, thus allowing rapid dynamic neurotransmitter detection. The cis aptamer/trans ligand configuration is not applicable to anionic ligands as they will be prevented from entering the nanopore from the trans side by the voltage that drives the aptamer into the nanopore from the cis side. In spite of this limitation, the approach is still applicable to most cationic small molecules, including all the neurotransmitters. Recently, an aptamer-immobilized field effect transistor (FET) has been applied in neurotransmitter detection by electrically measuring the aptamer conformation change caused by ligand binding. By comparison, the nanopore has a potential to detect neurotransmitters by discriminating and counting single aptamer molecule conformation transitions with a high signal/noise ratio.

Currently, the limitation of this nanopore sensor for real-time small molecule sensing is the limit of detection (LOD). LOD is the lowest ligand concentration at which the ligand binding events counted by the nanopore reaches a resolvable frequency. In dopamine detection, it was observed that the frequency of AL blocks f in 100 nM dopamine was 0.11 s⁻¹ (average of 1 AL block every 9 seconds, as shown in FIG. 2F). If the minimal observable frequency, for example, is set at 0.1 s⁻¹, in order to observe dozens of events in an approximately 10-minute recording, then the LOD is 100 nM. This LOD, however, is not low enough to detect neurotransmitters that are in the 1-10 nM range in tissue. The limiting factor is the low frequency of dopamine binding events at low concentrations. This factor also causes high dissociation constants (K_{d}) measured by the nanopore for the three aptamers, which are higher than what was found in free solution by two orders of magnitude. At ligand concentrations well below the K_{d} value, the principal determinant of the observable event frequency is the ligand-association rate, kₒₙ. kₒₙ in the nanopore environment is likely much smaller than in free solution to account for why the K_{d} value calculated in our nanopore system (k_{off}/kₒₙ) is much higher than reported in free solution. The ligand concentration in the nanopore lumen likely deviates considerably from that in the bulk trans solution due to the local charge environment within the nanopore. It is also likely that movement of the charged ligands through the nanopore driven by the electric field has a profound, and possibly complex, effect on the ligand-association rate. Future work to increase sensitivity will focus on increasing the frequency of nanopore ligand-binding events. One strategy is to enhance the cationic ligand flow from the trans entrance by manipulating the local charge distribution via site-specific nanopore mutations, pH and ion strength. These factors are believed to regulate ligand flow through electrostatic interaction, ion selectivity, and/or electroosmotic flow. Another strategy is to fine tune the kinetics of aptamer-ligand interactions to enhance the ligand binding rate (kₒₙ) by site-specific sequence alteration and nucleotide modification. Finally, the optimized sensor can be integrated within nanopore arrays in commercial devices for high-throughput neurochemical detection or drug screening; and the protein nanopore can also be incorporated into the tips of micropipette probes (pre-filled with aptamer) amenable to micromanipulation to achieve high spatial- and temporal-resolution in quantifying ligand concentrations.

### Nanopores

The disclosure provides for nanopores, particularly transmembrane nanopores. Any suitable nanopore or combination of nanopores may be used according to the disclosure. Generally, nanopores can be grouped into two categories: ‘biological pores’ which are pore proteins such as alpha hemolysin (aHL) or Mycobacterium smegmatis porin A (MspA) and are generally embedded in a phospho-lipid bilayer; and solid-state pores, which are nanometer-scale holes drilled into thin membranes of silicon nitride, graphene, molybdenum disulfide, etc. The nanopores of the disclosure often cross the entire membrane so that hydrated ions may flow from one side of the membrane to the other side of the membrane. However, the nanopore does not have to cross the membrane. It may be closed at one end. For instance, the nanopore may be a well, gap, channel, trench or slit in the membrane along which or into which hydrated ions may flow.

Any suitable nanopore may be used in the systems, methods, and apparatuses disclosed herein, such as biological or artificial nanopores. Suitable nanopores include, but are not limited to, protein nanopores, polynucleotide nanopores and solid-state nanopores. In a preferred embodiment, the nanopore is a transmembrane protein nanopore. A transmembrane protein nanopore comprises a polypeptide or a collection of polypeptides that permits hydrated ions, such as analyte, to flow from one side of a membrane to the other side of the membrane. In the present disclosure, the transmembrane protein nanopore is capable of forming a nanopore that permits hydrated ions driven by an applied potential to flow from one side of the membrane to the other. The transmembrane protein nanopore preferably permits a polynucleotide, such as DNA or RNA, to be moved through the nanopore.

The protein nanopore may be a monomer or an oligomer. The nanopore is preferably made up of several repeating subunits, such as at least 6, at least 7, at least 8, at least 9, at least 10, at least 11, at least 12, at least 13, at least 14, at least 15, or at least 16 subunits. The nanopore is preferably a hexameric, heptameric, octameric or nonameric nanopore. The nanopore may be a homo-oligomer or a hetero-oligomer. In a particularly preferred embodiment, the nanopore is octameric.

The protein nanopore typically comprises a barrel or channel through which the ions may flow. The subunits of the nanopore typically surround a central axis and contribute strands to a transmembrane β barrel or channel or a transmembrane α-helix bundle or channel.

The barrel or channel of the transmembrane protein nanopore typically comprises amino acids that facilitate interaction with a ligand, such as nucleotides, polynucleotides or nucleic acids. These amino acids are preferably located near the center of the barrel or channel, although they can be located at any part of the nanopore. The transmembrane protein nanopore typically comprises one or more positively charged amino acids, negatively charged amino acids, or aromatic amino acids, These amino acids can facilitate the interaction between the nanopore and the ligands and/or aptamers.

Nanopores for use in accordance with the disclosure can be derived from β-barrel nanopores or α-helix bundle nanopores. β-barrel nanopores comprise a barrel or channel that is formed from β-strands. Suitable β-barrel nanopores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other nanopores, such as lysenin. α-helix bundle nanopores comprise a barrel or channel that is formed from α-helices. Suitable α-helix bundle nanopores include, but are not limited to, inner membrane proteins and a outer membrane proteins, such as WZA and ClyA toxin.

The nanopore is preferably a Mycobacterium smegmatis porin (Msp) or derived from Msp. Such a nanopore will be oligomeric and typically comprises 7, 8, 9 or 10 monomers derived from Msp. The nanopore may be a homo-oligomeric nanopore derived from Msp comprising identical monomers. Alternatively, the nanopore may be a hetero-oligomeric nanopore derived from Msp comprising at least one monomer that differs from the others. Preferably the nanopore is derived from MspA or a homolog or paralog thereof.

MspA is an octameric pore with a goblet-like conformation comprising a large interior cavity and a thin narrow hydrophobic constriction at one end. The internal diameter varies from 4.8 nm at the cis end (external to the cell) and 1.2 nm at the trans mouth (internal to the cell). More particularly, the MspA nanopore comprises a cis vestibule and a trans vestibule. The cis vestibule comprises a relatively large goblet-shaped lumen/cavity while the trans end of the pore comprises a constriction that is a relatively short, restrictive, sensing zone.

Unlike other types of nanopores, such as aHL and aerolysin, wild-type MspA does not form an ion-conducting channel that has the properties for biosensing. Rather the channel is typically mutated to produce a pore that is both thermally and chemically stable. The mutated channel used for sensing is preferably cation selective. Beneficially, the internal cavity of MspA is large compared to other nanopores, with its conductance higher as a result. Additionally, the thin, narrow constriction of approximately 1.2 nm in diameter and only 0.6 nm thick near the trans mouth of the pore beneficially restricts the sensing location of the pore to this region, thereby effectively restricting the motion of small molecules (e.g., DNA) for sequence reading.

### Mutant Nanopores

In an embodiment, the nanopore is a mutant nanopore, such as one comprising a mutant Msp. A mutant Msp nanopore is a nanopore whose sequence varies (i.e., contains one or more variants) from that of a wild-type Msp and which retains the ability to form a pore. The mutant nanopore may be generated using any suitable method, such as targeted mutation/mutagenesis. Examples of such methods are found, for example, Butler et al., Single-Molecule DNA Detection with an Engineered MspA Protein Nanopore. Proc Natl Acad Sci USA 2008,105,20647-20652; and Craig et al., Revealing Dynamics of Helicase Translocation on Single-Stranded DNA Using High-Resolution Nanopore Tweezers. Proc NatlAcad Sci US A 2017, 114, 11932-11937, both of which are herein incorporated by reference in their entirety.

More generally, the mutant nanopore may contain one or more specific modifications to facilitate nucleotide discrimination. It may also contain other non-specific modifications as long as they do not interfere with pore formation. A number of non-specific side chain modifications are known in the art and may be made to the side chains of the monomer derived from Msp.

SEQ ID NO: 8 is the wild-type MspA monomer. The MspA mutant may comprise a variant of the amino acid sequence shown in SEQ ID NO: 8. The variant may comprise any of the mutations in the MspA monomer. A variant is a polypeptide that has an amino acid sequence which varies from that of SEQ ID NO: 8 and which retains its ability to form a pore. A variant typically contains the regions of SEQ ID NO: 8 that are responsible for pore formation. The pore forming ability of Msp, which contains a β-barrel, is provided by β-sheets in each subunit. A variant of SEQ ID NO: 8 typically comprises the regions in SEQ ID NO: 8 that form β-sheets. One or more modifications can be made to the regions of SEQ ID NO: 8 that form β-sheets as long as the resulting variant retains its ability to form a pore. A variant of SEQ ID NO: 8 preferably includes one or more modifications, such as substitutions, additions or deletions, within its α-helices and/or loop regions.

The variant preferably comprises one or more modifications in a part of the variant which interacts with the polynucleotide binding protein. This improves the movement and/or binding capacity of a target polynucleotide with respect to a transmembrane pore comprising the variant when the movement is controlled by a polynucleotide binding protein. Importantly, the variant preferably comprises one or more modifications enabling a nucleic acid scaffold, such as an aptamer, to non-covalently coordinate with the inside of the MspA variant.

Wild-type MspA comprises aspartic acid (D) at positions 118 and 134 and glutamic acid (E) at position 139. The one or more modifications to the wild-type MspA preferably reduce the net negative charge at one or more of positions 118, 126, 134 and 139 through the substitution of one or more neutral polar and/or positively charged amino acids. In particular, the variant preferably does not comprise aspartic acid (D) or glutamic acid (E) at one or more of positions 90, 91, 93, 118, 134 and/or 139. The variant preferably does not comprise aspartic acid (D) or glutamic acid (E) at any of the combination of positions 90, 91, 93, 118, 134 and/or 139. The variant more preferably comprises arginine (R), asparagine (N), or lysine (K) at one or more of positions 118, 126, 134 and 139, such as any of the combinations of positions 118, 126, 134 and 139 disclosed above. The variant most preferably comprises D90N, D91N, D93N, D118R, D134R and/or E139K.

More particularly, MspA mutant M2 (“MspA-M2”) as shown in SEQ ID NO: 7 substitutes six negatively charged amino acids (D and E) in the lumen of the wild type MspA by neutral polar (N) and positively charged amino acids (R and K), specifically, D90N/D91N/D93N/D118R/D134R/E139K. Preferably, MspA-M2 is used to construct M2-based variants at the R118 and R134 sites to enable non-covalent aptamer docking inside the protein nanopore. In a preferred embodiment, the M2-based variants are M2-R118N/R134N (referred to as “M3” and shown in SEQ ID NO: 4) M2-R118N (referred to as “M8” and shown in SEQ ID NO: 6) and M2-R134N (referred to as “M7” and shown in SEQ ID NO: 5).

Over the entire length of the amino acid sequence of SEQ ID NOS: 4-8, a variant will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NOS: 4, 5, 6, 7, or 8 over the entire sequence. There may be at least 80%, for example at least 85%, 90% or 95%, amino acid identity over a stretch of 100 or more, for example 125, 150, 175 or 200 or more, contiguous amino acids.

In an embodiment, the one or more variants in M2, M3, M7, and/or M8 introduce one or more charged “rings” in the protein nanopore. In an embodiment, the M2-based variant comprises a positively charged ring (i.e., cationic ring) at the R118 and/or R134 sites. The one or more rings may be positioned anywhere in the nanopore. In an embodiment, the R118 ring is positioned in the middle of the pore and the R134 ring is positioned near the pore opening, such as the cis entrance, as described in Wendel et al., Adaptation of Mycobacterium smegmatis to an Industrial Scale Medium and Isolation of the Mycobacterial PorinMspA. Open Microbial J2013, 7, 92-98, which is herein incorporated by reference in its entirety.

Any of the proteins described herein, such as the transmembrane protein nanopores, may be modified to assist their identification or purification, for example by the addition of histidine residues (a his tag), aspartic acid residues (an asp tag), a streptavidin tag, a flag tag, a SUMO tag, a GST tag or a MBP tag, by a label, or by the addition of a signal sequence to promote their secretion from a cell where the polypeptide does not naturally contain such a sequence. Alternatively, the apparatuses, systems, and methods may be label free.

Any of the proteins described herein, such as the transmembrane protein nanopores, may be made synthetically or by recombinant means, using standard methods known in the art. Polynucleotide sequences encoding a nanopore or construct may be derived and replicated using standard methods in the art. For example, polynucleotide sequences encoding a nanopore or construct may be expressed in a bacterial host cell using standard techniques in the art. The nanopore may be produced in a cell by in situ expression of the polypeptide from a recombinant expression vector. The nanopore may be produced in large scale following purification by any protein liquid chromatography system from protein producing organisms or after recombinant expression.

### Membrane

The systems, methods, compositions, and apparatuses of the disclosure preferably utilize a membrane, in particular a membrane comprising one or more transmembrane nanopores. Any membrane may be used in accordance with the disclosure. In an embodiment, the membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. The amphiphilic molecules may be chemically modified or functionalized to facilitate coupling of the ligand or one or more adapters.

Non-naturally occurring amphiphiles and amphiphiles which form a monolayer are known in the art and include, for example, block copolymers. Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. The block copolymer may possess amphiphilic properties and may form a structure that mimics a biological membrane. The block copolymer may be a diblock (consisting of two monomer sub-units) but may also be constructed from more than two monomer sub-units to form more complex arrangements that behave as amphiphiles. The copolymer may comprise a triblock, tetrablock or pentablock copolymer.

Block copolymers may also be constructed from sub-units that are not classed as lipid sub-materials. For example, a hydrophobic polymer may be made from siloxane or other non-hydrocarbon-based monomers. The hydrophilic sub-section of block copolymer can also possess low protein binding properties, which allows the creation of a membrane that is highly resistant when exposed to raw biological samples. This head group unit may also be derived from non-classical lipid head-groups. Triblock copolymer membranes also have increased mechanical and environmental stability compared with biological lipid membranes, for example a much higher operational temperature or pH range. The synthetic nature of the block copolymers provides a platform to customize polymer-based membranes for a wide range of applications.

The amphiphilic layer may be a monolayer or a bilayer. The amphiphilic layer is typically planar. The amphiphilic layer may be curved. The amphiphilic layer may be supported. The amphiphilic layer may be concave.

Ina preferred embodiment, the membrane comprises a lipid bilayer. Lipid bilayers provide a variety of suitable uses. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. In an embodiment, the lipid bilayer is preferably a planar lipid bilayer.

Methods for forming lipid bilayers are known in the art. Suitable methods are disclosed in the Examples, for example preparation of a lipid bilayer membrane (1,2-diphytanoyl-sn-glycero-3-phosphocholine) formed over a 100-150 μm orifice in the center of a Teflon film that partitioned between cis and trans recording solutions, wherein the solutions in both cis and trans chambers contain 1 M KCl buffered with 10 mM Tris (pH 7.4). Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface. For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement. Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

More generally, a lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such as surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester.

The lipids can also be chemically modified or functionalized. The head group or the tail group of the lipids may be chemically modified. Suitable lipids whose head groups have been chemically-modified include, but are not limited to, PEG-modified lipids, such as 1,2-Diacyl-sn-Glycero-3-Phosphoethanolamine-N-[Methoxy(Polyethylene glycol)-2000]; functionalized PEG Lipids, such as 1,2-Distearoyl-sn-Glycero-3 Phosphoethanolamine-N-[Biotinyl(Polyethylene Glycol)2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; ether linked lipids, such as 1,2-Di-O-phytanyl-sn-Glycero-3-Phosphocholine; and methylated lipids, such as 1,2-diphytanoyl-sn-glycero-3-phosphocholine.

The amphiphilic layer, for example the lipid composition, typically comprises one or more modifications or functionalization, such as methylation. In a preferred embodiment, the lipid membrane is a bilayer lipid membrane, specifically a phospholipid membrane containing the tetramethylated long-chain (16:0) diphytanic acid at the sn-1 and sn-2 positions.

### Ligands

The systems, methods, compositions, and apparatuses described herein preferably comprise one or more small molecules to be identified, measured, sequenced, or otherwise analyzed. More particularly, the disclosure provides for the use of one or more ligands, in particular small molecule ligands that selectively bind with aptamers positioned within the nanopores described herein. As used herein, the term “ligand” broadly refer to a molecule or atom that binds, preferably reversibly, preferably to an aptamer as described herein, and preferably wherein the binding disrupts an ion current passing through the nanopore to characteristically vary as the aptamer undergoes conformational changes, resulting in a sequence of current fluctuations that report binding and release of single ligand molecules. The term “ligand” as provided herein should be broadly construed and may be used interchangeably with the term “analyte,” i.e., any constituent that is the subject of analysis.

Exemplary ligands include but are not limited to neurotransmitters, antibodies, receptors, peptides, nucleic acids, hormones, metabolites, antibiotics, therapeutic compounds, and/or diagnostic compounds that bind to a target aptamer. More particularly, the ligand may comprise one or more of metal ions, inorganic salts, polymers, amino acids, peptides, polypeptides, proteins, nucleotides, oligonucleotides, polynucleotides, dyes, bleaches, pharmaceuticals, diagnostic agents, recreational drugs, explosives and/or environmental pollutants. The ligand may be a biomarker. In some embodiments, the ligand comprises two or more molecules of the same type, such as two or more proteins, two or more nucleotides or two or more pharmaceuticals. In other embodiments, the ligand comprises two or more molecules of different types, such as one or more proteins, one or more nucleotides and one or more pharmaceuticals.

In one embodiment, the ligand is selected from amino acids, peptides, polypeptides, proteins, nucleotides, oligonucleotides and/or polynucleotides. The amino acids, peptides, polypeptides, proteins, nucleotides, oligonucleotides and/or polynucleotides can be naturally occurring or non-naturally occurring. The polypeptides or proteins can include within them synthetic or modified amino acids. The proteins can be enzymes, antibodies, hormones, biomarkers, growth factors or growth regulatory proteins, such as cytokines. The cytokines may be selected from interleukins, such as IL-1, IL-1, IL-2, IL-4, IL-5, IL-6, IL-10, IL-12 and IL-13, interferons, such as IFN-g, and other cytokines such as TNF-a. The proteins may be bacterial proteins, fungal proteins, virus proteins or parasite-derived proteins. A nucleotide typically contains a nucleobase, a sugar and at least one phosphate group. Nucleobases include, but are not limited to, purines and pyrimidines and more specifically adenine, guanine, thymine, uracil and cytosine. The sugar may comprise a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

More particularly, suitable nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate.

The nucleotides may be abasic (i.e. lack a nucleobase). The nucleotides may contain additional modifications. In particular, suitable modified nucleotides include, but are not limited to, 2′amino pyrimidines (such as 2′-amino cytidine and 2′-amino uridine), 2′-hyrdroxyl purines (such as, 2′-fluoro pyrimidines (such as 2′-fluorocytidine and 2′fluoro uridine), hydroxyl pyrimidines (such as 5′-a-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

Oligonucleotides are short nucleotide polymers which typically have 50 or fewer nucleotides, such 40 or fewer, 30 or fewer, 20 or fewer, 10 or fewer or 5 or fewer nucleotides. The oligonucleotides may comprise any of the nucleotides discussed herein, including the abasic and modified nucleotides.

The polynucleotides may be single stranded or double stranded. At least a portion of the polynucleotide may be double stranded. The polynucleotides can be nucleic acids, such as deoxyribonucleic acid (DNA) or ribonucleic acid (RNA). In one embodiment, the ligand comprises microRNA (miRNA). The polynucleotides can comprise one strand of RNA hybridized to one strand of DNA. The polynucleotides may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains. The polynucleotides may comprise any of the nucleotides discussed herein, including the modified nucleotides.

The polynucleotides can be any length. For example, the polynucleotides can be at least 10, at least 50, at least 100, at least 150, at least 200, at least 250, at least 300, at least 400 or at least 500 nucleotides or nucleotide pairs in length. The polynucleotides can be 1000 or more nucleotides or nucleotide pairs, 5000 or more nucleotides or nucleotide pairs in length or 100000 or more nucleotides or nucleotide pairs in length.

The ligand may be associated with a particular phenotype or with a particular type of cell. For instance, the ligand may be indicative of a particular species of bacteria, a virus, a fungus or a parasite. In one embodiment, the ligand comprises a biomarker that can be used to diagnose or prognose a disease or condition. The biomarkers may be any of the molecules described herein, such as proteins or polynucleotides.

In one embodiment, the ligand comprises a neurotransmitter. Examples of suitable neurotransmitters include acetylcholine, serotonin, dopamine, epinephrine, norepinephrine, theophylline, nucleotides such as ATP, amino acids such as glutamate, aspartate and d-aminobutyric acid, and/or enkephalins.

In a preferred embodiment, ligand is preferably a small molecule. The ligand may comprise a small molecule having a molecular weight of up to 1,000 Daltons and/or a length of less than about 1.2 nm. In another embodiment, the ligand may be cationic or anionic. Preferably, the ligand is a cationic small molecule. The ligand may be a short, single strand nucleic acid sequence comprising DNA, RNA, and/or nucleic acids or analogs. In a preferred embodiment, the ligand comprises dopamine, serotonin, and/or theophylline.

Ligands may be selected to bind to a specific target aptamer. In certain embodiments, two or more aptamers may bond with the same nanopore. For example, 2 or more ligands, including 2, 3, 4, 5, 6, 7, 8, 9, 10 or more ligands may be bonded with an aptamer positioned in the nanopore. The ligand and aptamer may bond or coordinate through any suitable mechanism, for example covalent bonding, non-covalent bonding, van der Waals forces, hydrogen bonding, electrostatic forces, or the like.

### Aptamer

The systems, methods and apparatuses may further include one or more aptamers. As used herein, the term “aptamer” is to be understood broadly as any molecule or compound that binds, preferably reversibly, with one or more ligands and/or the nanopore as described herein, where preferably the conformational changes of such binding and release resulting in a disruption of an ion current passing through a nanopore and causing a sequence of current fluctuations. Thus, the aptamer may comprise a sequence of DNA, RNA, XNA, or a peptide, a riboswitch aptamer, an RNA element (e.g., the HIV trans-activation response (TAR) element), an RNA structure or entry site (e.g., the hepatitis C virus (HCV) internal ribosome entry site), a frameshifting element, (e.g., the SARS-CoV-2 frameshifting element), and/or RNA repeats. Preferably, the aptamer comprises a single strand of nucleic acids. The aptamer may carry a charge. For example, the aptamer is cationic or anionic. In a preferred embodiment, the aptamer is cationic. In an embodiment, the aptamer is capable of non-covalent bonding with the nanopores and can stably reside in the pore while transitioning among multiple conformations.

More particularly, the aptamer may be a peptide aptamer or an oligonucleotide aptamer. The aptamer can be any length, for example, between about 15 and about 120 nucleotides in length, such as from about 15 to about 50, from about 20 to about 40 or from about 25 to about 30 amino acids or nucleotides in length, inclusive of all integers within these ranges.

In a preferred embodiment, the aptamer comprises an oligonucleotide. In a further embodiment, the aptamer is an oligonucleotide having the sequence shown in SEQ ID NO: 1 and has the ability to bind with a ligand comprising dopamine. In an embodiment, the aptamer is an oligonucleotide having the sequence shown in SEQ ID NO: 2 and has the ability to bind with a ligand comprising serotonin. In a still further embodiment, the aptamer is an oligonucleotide having the sequence shown in SEQ ID NO: 3 and has the ability to bind with a ligand comprising theophylline.

The aptamer comprises one or more motifs that may serve a functional purpose, e.g., may present a binding site for a ligand, wherein the binding of the ligand induces one or more conformational changes in the aptamer. Suitable motifs include, without limitation, a loop, β-turn, coiled region, helix, triple helix, four helix bundle, hairpin loop, multibranch loop, single nucleotide internal loop, Greek key motif, parallel β-helix, β-roll, G-quadruplex, β-bulge, β-bulge loop, TIM barrel, Zinc finger motif, β-bend ribbon, β-hairpin, β-sheet, β-sandwich, catgrip, collagen helix, F-box protein, gamma helix, granin, helix-turn-helix, heptad repeat, leucine zipper, omega loop, nest, niche, PHD finger, pi helix, polyproline helix, protein i-sites, Schellman loop, short linear motif, ST motif, ST staple, ST turn, sterile alpha motif, TMPad, transmembrane domain, Walker motif, YGL motif, RING finger domain, Rossmann folder, recognition sequence, or a combination thereof. The motif may comprise any suitable structural motif or supersecondary structure.

In a preferred embodiment, the aptamer comprises a neurotransmitter-binding aptamer. Particularly preferred neurotransmitter-binding aptamers comprise dopamine-binding, serotonin-binding, and/or theophylline-binding aptamers.

The one or more aptamers may be bound or docked in the nanopore through any suitable mechanism, such as covalent or non-covalent attachment. The nanopore and ligand may be chemically fused or genetically fused. In a preferred embodiment, the nanopore system described herein comprises at least one nanopore having one or more aptamers docked in the nanopore non-covalently, such that the non-covalent coordination between the aptamer and nanopore suspends the aptamer in the nanopore cavity. In such cases, where the aptamer is “suspended,” it is fully exposed to the surrounding ion pathway, enabling the ion current to sensitively change with the aptamer conformation. This suspension also means that the aptamer beneficially does not block the ligand pathway at the narrow trans entrance of the nanopore, thereby allowing the ligand to flow through the pore from the trans entrance to interact with the suspended aptamer from different directions, regardless of the orientation of the aptamer's binding site. Furthermore, the suspension of the aptamer makes available more potential binding sites for one or more additional or similar ligands.

### Adapters

The systems, compositions, methods, and apparatuses described herein may optionally utilize one or more adapters. The term adapter as used herein broadly encompasses any atom or molecule that serves to facilitate or enable ligand detection, enable or improve ligand-aptamer bonding, enable or improve aptamer-nanopore bonding, and/or to functionalize the nanopore or membrane, including any tag, label, linker, spacer, microparticle, or molecular probe.

However, in a preferred embodiment, the aptamer and the nanopore are free of adapters. Preferably, an unmodified aptamer is able to engage in non-covalent docking within the lumen of the nanopore. Beneficially, according to this embodiment, the location-specific, non-covalent docking of the aptamer in the pore “suspends” the aptamer centrally in the lumen cavity of the nanopore, at which location the aptamer transitions between different conformations that result in specific, characteristic, and measurable changes in a current being applied to the nanopore. This feature beneficially enables single-molecule detection of the aptamer's conformational transitions, which is a significant improvement because existing approaches are not sensitive enough to detect small conformational changes of a nucleic acid scaffold (e.g., small molecule aptamers) upon the binding of a ligand. Furthermore, the non-covalent coordination of the aptamer to the nanopore without requiring an adapter overcomes the significant technical and cost hurdle of custom fabrication of nanopores with attached adapters (e.g., molecular probes). Existing non-covalent solutions that may permit ligand detection without a molecular probe do not have a pore wide enough to accommodate most nucleic acid motifs. Thus, the solution described herein, a nucleic acid-docked nanopore capable of non-covalently bonding with an unmodified aptamer and capable of discriminating and continuously recording small molecule-regulated nucleic acid dynamic conformational variation, overcomes these problems.

It will be appreciated that the adapter-free nanopore system described herein may be optionally combined with one or more other methods to allow detection of multiple ligands or to achieve multiple desired functions simultaneously. For example, the methods may be for detecting 2 or more, such as 5 or more, 10 or more, 50 or more, 100 or more, for example from 200 to 500, 500 or more, for example from 600 to 1000, or at least 1000, for example from 1000 to 10000, different ligands.

When utilized, the adapter often has an effect on the physical or chemical properties of the pore, ligand, aptamer, or membrane such that it improves its interaction with one or more other components of the nanopore systems described herein. For example, when the adapter is bound inside the pore, the adapter may alter the charge of the barrel or channel of the pore to improve pore-aptamer interactions.

Accordingly, the adapter may comprise one or more chemical groups that facilitate interactions between one or more of the nanopore, the ligand, the aptamer, or the membrane. The one or more chemical groups conduct this interaction via non-covalent interactions, such as hydrophobic interactions, hydrogen bonding, Van der Waal's forces, 71-cation interactions and/or electrostatic forces. The molecular adapter may be covalently attached to the nanopore, ligand, aptamer, or membrane. The adapter can be covalently attached using any method known in the art. For example, adapters are often attached via chemical linkage.

The molecular adapter may comprise a cyclic molecule, a cyclodextrin, a species that is capable of hybridization, a DNA binder or interchelator, a peptide or peptide analogue, a synthetic polymer, an aromatic planar molecule, a small positively charged molecule or a small molecule capable of hydrogen-bonding.

When the adapter is a cyclic molecule attached to the nanopore, cyclic adapter preferably has the same symmetry as the pore. The adapter preferably has eight-fold symmetry since Msp typically has eight subunits around a central axis. Such suitable adapters include, but are not limited to, cyclodextrins, cyclic peptides and cucurbiturils. The adapter is preferably a cyclodextrin or a derivative thereof. More particularly, the adapter may comprise heptakis-6-amino-β-cyclodextrin (am₇-PCD), 6-monodeoxy-6-monoamino-β-cyclodextrin (am₁-βCD) or heptakis-(6-deoxy-6-guanidino)-cyclodextrin (gu₇-βCD). The specific type of adapter may be selected based on any desired function. For example, the guanidino group in gu₇-βCD has a much higher pKa than the primary amines in am₇-βCD and so it is more positively charged. This gu₇-βCD adapter may be used to increase the dwell time of the nucleotide in the pore, to increase the accuracy of the residual current measured, as well as to increase the base detection rate at high temperatures or low data acquisition rates. More suitable adapters include γ-cyclodextrins, which comprise 8 sugar units (and therefore have eight-fold symmetry). The γ-cyclodextrin may contain a linker molecule or may be modified to comprise all or more of the modified sugar units used in β-cyclodextrin.

The adapter may comprise a label that permits detection. Any suitable label may be used, including but not limited to, fluorescent molecules, radioisotopes, enzymes, antibodies, antigens, polynucleotides, or a combination thereof.

The adapter may be modified to include a genetic tag or chemical tag, for example to assist in identification or purification. Examples include but are not limited to histidine residues (a his tag), a polyhistidine-tag (hexa histidine-tag, 6×His-tag, His6 tag or His-tag), Ni-NTA, biotin, aspartic acid residues (an asp tag), a streptavidin tag, a flag tag, a SUMO tag, a GST tag, a MBP tag, an oligonucleotide, a polynucleotide (such as DNA, RNA, PNA, GNA, TNA or LNA), carboxyl groups, quaternary amine groups, thiol groups, azide groups, alkyne groups, DIBO, lipid, FLAG-tag (FLAG octapeptide, polynucleotide binding proteins, peptides, proteins, antibodies or antibody fragments, or a combination thereof.

### Nanopore Systems

Disclosed herein are nanopore systems capable of real-time, label-free/adapter-free characterization of dynamic aptamer-small molecule (e.g, ligand) interactions. In its basic implementation, a nanopore system comprises one or more pores within a membrane that form a cell. This membrane divides a salt solution into two wells or chambers called ‘cis’ and ‘trans’ wherein the cis chamber is external to the cell and the trans chamber is internal to the cell. The salt solution in the cis and trans chambers can be further be defined based on location, e.g., the cis solution is located in the cis chamber/side of the membrane and trans solution is located in the trans chamber/side of the membrane. When a voltage is applied across this membrane, an ion current flows through the pore. The magnitude of this ion current is the primary signal. Molecules of interest (i.e., DNA, RNA, peptides, nanoparticles, etc.) are drawn towards the pore and then through it by the electric field. As the molecules traverse the pore and/or as the molecules interact with each other inside the pore, they alter the ion current flowing through the pore. Depending on the degree to which the ion current is blocked, various properties of the molecule and its movement through the pore can be inferred.

The nanopore systems described herein preferably comprise one or more of: (a) a membrane; (b) a nanopore transversing the membrane and comprising a cis vestibule and a trans vestibule, wherein the cis vestibule comprises a lumen and the trans vestibule comprises a constriction; (c) a cis chamber located on the side of the membrane closest to the cis vestibule; (d) a trans chamber located the side of the membrane closest to the trans vestibule; (e) an aptamer docked in the nanopore such that the aptamer is suspended in the lumen via non-covalent bonding between the aptamer and the nanopore; (f) a ligand capable of binding with the aptamer; (g) an electrical circuit capable of applying an electric field to the nanopore system and generating an ion current; and (h) an apparatus capable of taking one or more measurements of the ion current; wherein the one or more measurements are indicative of one or more characteristics of the aptamer, the ligand, an interaction between the aptamer and the ligand, or an interaction between the aptamer and the nanopore.

Regarding (a), the membrane preferably comprises a lipid bilayer. Lipid bilayers provide a variety of suitable uses. For example, lipid bilayers can be used for in vitro investigation of membrane proteins by single-channel recording. In a preferred embodiment, the membrane is a phospholipid membrane comprising the tetramethylated long-chain (16:0) diphytanic acid at the sn-1 and sn-2 positions. In a still further preferred embodiment, the membrane is a lipid bilayer comprised of 1,2-diphytanoyl-sn-glycero-3-phosphocholine.

For (b), the nanopore, is preferably a transmembrane biological pore comprised of alpha hemolysin (aHL) or Mycobacterium smegmatis porin A (MspA), Mycobacterium smegmatis porin B (MspB), Mycobacterium smegmatis porin C (MspC), or Mycobacterium smegmatis porin D (MspD). In a preferred embodiment, the nanopore is comprised of a mutant MspA. In a further embodiment, the mutant MspA comprises a mutation at positions 90, 91, 93, 118, 134, and/or 139, wherein negative charges are removed from positions 90, 91, 93, 118, 134, and/or 139 compared to a wild-type MspA, and/or wherein a negatively charged amino acid at positions 90, 91, 93, 118, 134, and/or 139 of a wild-type MspA is replaced by a positively charged amino acid or a neutral polar amino acid. In a further embodiment, the mutant MspA comprises D90N, D91N, D93N, D118R, D134R and/or E139K. In a still further embodiment, the mutant MspA comprises a mutant as shown in one or more of SEQ ID NO: 7, SEQ ID NO: 4, SEQ ID NO:6, AND SEQ ID NO: 5. In an embodiment, over the entire length of the amino acid sequence of SEQ ID NOS: 4-7, the mutant MspA will preferably be at least 50% homologous to that sequence based on amino acid identity. More preferably, the mutant MspA will be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NOS: 4, 5, 6, or 7 over the entire sequence.

For (c) and (d), cis chamber and the trans chamber preferably comprise a solution and is capable of carrying charges, such as solutions of metal salts, for example alkali metal salt, halide salts, for example chloride salts, such as alkali metal chloride salt. In an embodiment, the carrier solution comprises potassium chloride (KCl), sodium chloride (NaCl), cesium chloride (CsCl) or a mixture of potassium ferrocyanide or potassium ferricyanide. The chambers may also comprise a buffer. Preferably, the buffer comprises a phosphate buffer, HEPES, and/or Tris-HCl.

Regarding (e) the aptamer may comprise a sequence of DNA, RNA, XNA, a peptide, a riboswitch aptamer, an RNA element, an RNA structure, an RNA entry site, a frameshifting element, and/or RNA repeats. The aptamer may carry a charge. Preferably, the aptamer is cationic. In an embodiment, the aptamer is capable of non-covalent bonding with the nanopore and can stably reside in the pore while transitioning among multiple conformations. Preferably, the aptamer comprises an oligonucleotide. In a further embodiment, the aptamer is an oligonucleotide having the sequence shown in SEQ ID NO:1 and has the ability to bind with a ligand comprising dopamine. In an embodiment, the aptamer is an oligonucleotide having the sequence shown in SEQ ID NO:2 and has the ability to bind with a ligand comprising serotonin. In a still further embodiment, the aptamer is an oligonucleotide having the sequence shown in SEQ ID NO:3 and has the ability to bind with a ligand comprising theophylline.

For (f), the ligand preferably comprises a small molecule to be identified, measured, sequenced, or otherwise analyzed. The ligand may comprise a neurotransmitter, antibody, receptor, peptide, nucleic acid sequence, hormone, metabolite, antibiotic, therapeutic compound, biomarker, and/or diagnostic compound. In a preferred embodiment, the ligand comprises a neurotransmitter. Examples of suitable neurotransmitters include but are not limited to acetylcholine, serotonin, dopamine, epinephrine, norepinephrine, theophylline, nucleotides such as ATP, amino acids such as glutamate, aspartate and d-aminobutyric acid, and/or enkephalins. The ligand may comprise a small molecule having a molecular weight of up to 1,000 Daltons and/or a length of less than about 1.2 nm. In another embodiment, the ligand may be cationic or anionic. Preferably, the ligand is a cationic small molecule.

For (g), the electrical circuit may be any circuit or of any configuration capable of applying an electric field to the nanopore system. For example, the circuit may comprise a patch clamp or a voltage clamp. The circuit and overall nanopore system may be located or arranged in any suitable configuration, for example a silicon-based array of wells where each array comprises 128, 256, 512, 1024, 2000, 3000, 4000, 6000, 10000, 12000, 15000 or more wells. The electrical circuit generates an ion current. The ion current is typically generated via a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

Finally, regarding (h), any suitable apparatus may be used to take one or more measurements of the nanopore system. It will be appreciated that a variety of different types of measurements may be made. This includes without limitation: electrical measurements and optical measurements. Possible electrical measurements include current measurements, impedance measurements, tunnelling measurements as described in Ivanov et al., Nano Lett. 2011 Jan. 12; 11(1): 279-85 (which is herein incorporated by reference in its entirety), and FET measurements. Optical measurements may be combined with electrical measurements as described in Soni et al., Rev Sci Instrum. 2010 January; 81(1):014301, which is herein incorporated by reference in its entirety. Preferably, the measurement may be a transmembrane current measurement such as measurement of ionic current flowing through the pore. Electrical measurements may be made using standard single channel recording equipment such as described in Stoddart et al., Proc Natl Acad Sci, 12; 106(19):7702-7, and Lieberman et al, J Am Chem Soc. 2010; 132(50):17961-72, which are herein incorporated by reference in their entirety.

Significantly the nanopore systems described herein provide real-time, label-free/adapter-free detection of dynamic aptamer-small molecule (e.g., ligand) interactions. Beneficially, according to this embodiment, the location-specific, non-covalent docking of the aptamer in the pore “suspends” the aptamer in the lumen cavity, which enables single-molecule detection of the aptamer's conformational transitions. This is a significant improvement in the art because existing approaches are not sensitive enough to detect small conformational changes of a nucleic acid scaffold (e.g., small molecule aptamers) upon the binding of a ligand. Furthermore, the non-covalent coordination of the aptamer to the nanopore without requiring an adapter overcomes the significant technical and cost hurdle of custom fabrication of nanopores with attached adapters (e.g., molecular probes). Existing non-covalent solutions that may permit ligand detection without a molecular probe do not have a pore wide enough to accommodate most nucleic acid motifs. Thus, the solution described herein, a nucleic acid-docked nanopore system capable of non-covalently bonding with an unmodified aptamer and capable of discriminating and continuously recording small molecule-regulated nucleic acid dynamic conformational variation, overcomes these problems.

### Methods of Delivery

The disclosure further relates to methods of delivery, in particular methods of delivering ligands, aptamers, and/or samples to the membrane and nanopores.

The ligand and aptamer may be delivered towards the membrane and nanopore in any manner. The method preferably comprises positioning the ligand or aptamer near to or adjacent to the membrane and allowing the ligand or aptamer to move towards the membrane. The ligand or aptamer may be positioned any distance from the membrane, for instance about 500 μm from the membrane or closer, about 200 μm from the membrane or closer, about 100 μm from the membrane or closer, about 50 μm from the membrane or closer or about 30 μm from the membrane or closer.

The ligand and/or aptamer may move towards the membrane and into the nanopore in any manner. Preferably, the ligand or aptamer moves along an electrochemical gradient, diffusion gradient, hydrophilic gradient or hydrophobic gradient, or magnetic field. A gradient is an increase or decrease in the magnitude of a property observed when passing from one point or moment to another. The gradient may be generated using any suitable method. For example, a charged ligand or aptamer will generally move along an electrochemical gradient. Alternatively, ligands and aptamers may diffuse towards the membrane or flow in solution along a pressure gradient. A hydrophilic or hydrophobic ligand or aptamer will generally move along a hydrophilic or hydrophobic gradient.

In an embodiment, the ligand and/or aptamer move within an electrical field. Accordingly, in some embodiments the ligand and/or aptamer are delivered to the membrane using an electrical field, particularly when the ligand and/or aptamer are charged (e.g., a cationic ligand). The electrical field may be generated using any suitable method.

Alternatively, delivery may occur by causing the ligand and/or aptamer to move by applying pressure or flow, such as physical pressure or osmotic pressure. Gravity or a gravitational field may also be used to move the aptamer and/or ligand along the membrane and towards the nanopores.

More particularly, the disclosure provides for a method of delivering one or more ligands or aptamers to a membrane, particularly a membrane comprising one or more nanopores comprising: (a) providing one or more ligands and/or aptamers; (b) delivering the one or more ligands and/or aptamers towards a membrane, thereby delivering the one or more ligands and/or aptamers to the one or more nanopores; and (c) allowing the one or more ligands and/or aptamers to interact with the one or more nanopores. The delivery step may occur through the application of an electrochemical gradient, diffusion gradient, hydrophilic gradient, hydrophobic gradient, magnetic field, physical pressure, osmotic pressure, and/or gravity.

In some embodiments, the ligand is delivered to a first side of the membrane and the aptamer is delivered to a second side of the membrane. In another embodiment, the ligand and aptamer are delivered to the same side of the membrane. The ligand may be delivered to the cis and/or trans side of the membrane. The aptamer may also be delivered to the cis and/or trans side of the membrane. In a preferred embodiment, the ligand is delivered to and enters the nanopore from the trans side of the membrane. In a preferred embodiment, the aptamer is delivered to and enters the nanopore from the cis side of the membrane.

Methods of Real-Time, Label-Free Characterization of Aptamer Conformational Change Upon Binding and Unbinding with a Ligand

The disclosure further provides for methods of real-time characterization of aptamer conformational change upon binding and unbinding with the ligand and based on the use of a nucleic acid-docked nanopore that suspends the aptamer within the lumen of the nanopore. The methods of real-time characterization are part of a platform enabling multiple additional uses, such as assaying nucleic acid-small molecule interactions, small molecule sensing/detecting, screening of aptamer variant-ligand interactions and conformation changes, sequencing, diagnostics, gene switch design, and others.

### Methods of Non-Covalently Docking an Aptamer in a Nanopore

Disclosed herein are methods of non-covalently docking an aptamer in a nanopore to create a nucleic acid-docked nanopore capable of label-free/adapter-free, real-time characterization of ligands, aptamers, and their interactions.

In an embodiment, the methods of docking an aptamer in a nanopore comprise: (a) providing a mutant Mycobacterium smegmatis porin (Msp) nanopore comprising a cis vestibule comprising a lumen and a trans vestibule comprising a constriction that define a channel, wherein one or more negatively charged amino acids in the mutant Msp nanopore are substituted with one or more positively charged amino acids or neutral polar amino acids, such that the one or more positively charged amino acids or a neutral polar amino acids are distributed around the interior circumference of the mutant Msp nanopore thereby forming a ring; (b) delivering an aptamer to the mutant Msp nanopore; and (c) non-covalently binding the aptamer with the ring on the interior surface of the mutant Msp nanopore, wherein non-covalent binding suspends the aptamer in or near the focus of the ring (i.e., the center of the ring). In an alternative embodiment, the non-covalent binding suspends the aptamer at any position in the ring.

In an embodiment, the mutant Msp comprises a mutant MspA. In a further embodiment, the mutant MspA comprises a mutation at positions 90, 91, 93, 118, 134, and/or 139, wherein negative charges are removed from positions 90, 91, 93, 118, 134, and/or 139 compared to a wild-type MspA, and/or wherein a negatively charged amino acid at positions 90, 91, 93, 118, 134, and/or 139 of a wild-type MspA is replaced by a positively charged amino acid or a neutral polar amino acid. In a further embodiment, the mutant MspA comprises D90N, D91N, D93N, D118R, D134R and/or E139K. In a still further embodiment, the mutant MspA comprises a mutant as shown in one or more of SEQ ID NO: 7, SEQ ID NO: 4, SEQ ID NO:6, and SEQ ID NO: 5. In an embodiment, over the entire length of the amino acid sequence of SEQ ID NOS: 4-7, the mutant MspA will preferably be at least 50% homologous to that sequence based on amino acid identity. More preferably, the mutant MspA will be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NOS: 4, 5, 6, or 7 over the entire sequence.

In an embodiment, the ring on the interior circumference of the mutant Msp nanopore is located in the middle of the nanopore along the nanopore's vertical axis, i.e., equidistant between the cis opening and the trans opening of the nanopore. In an embodiment, the ring on the interior surface of the mutant Msp nanopore is located in the middle of the lumen along the nanopore's vertical axis, i.e., equidistant from the top of the lumen to the bottom of the lumen.

In an embodiment, the aptamer comprises a nucleobase sequence according to any one or more of SEQ ID NO: 1, SEQ ID NO:2, and SEQ ID NO: 3. In an embodiment, over the entire length of the amino acid sequence of SEQ ID NOS: 1-3 the aptamer nucleobase sequence will preferably be at least 50% homologous to that sequence based on nucleobase identity. More preferably, the aptamer nucleobase sequence will be at least 55%, at least 60%, at least 65%, at least 70%, at least 75%, at least 80%, at least 85%, at least 90% and more preferably at least 95%, 97% or 99% homologous based on nucleobase identity to the nucleobase sequence of SEQ ID NOS: 1-3 over the entire sequence.

Beneficially, as discussed herein, the non-covalent docking of aptamers in the mutant MspA nanopore results in the aptamer being suspended, preferably in the middle of the lumen cavity, where it is coordinated by charges on the ring. Essentially, the aptamer is held in suspension via non-covalent bonds by a nucleic acid-docked nanopore.

The aptamer binds to specific rings and those rings are positioned in specific, selectable positions within the mutant MspA nanopore. This docking configuration has several functions: i) the “suspended” aptamer is fully exposed to the surrounding ion pathway, enabling the ion current to sensitively change with the aptamer conformation; ii) the aptamer does not block the ligand pathway at the narrow trans entrance, allowing the ligand to flow through the pore from the trans side to interact with the suspended aptamer from different directions, regardless of the orientation of the aptamer's ligand binding site; and iii) the multiple blocking levels in the aptamer signature are confirmed to be generated by different conformations, rather than different locations of the aptamer in the pore. Thus, the ability to continuously observe aptamer dynamic conformational transitions originates from the aptamer docking configuration in the nanopore, which should not only stably hold the aptamer in the pore, but also enables the nanopore current to be sensitively modulated by the aptamer conformation.

### Methods of Ligand Characterization

The method of the disclosure preferably involves characterizing a ligand. The ligand is delivered to the membrane using the methods described herein, and nucleic acid-docked nanopores and aptamers described herein are used to characterize the ligand.

After delivery, the method comprises (a) allowing the ligand to interact with the nucleic acid-docked nanopore such that the ligand moves into the pore; (b) allowing the ligand to bind with an aptamer in the nanopore; and (c) taking one or more measurements as the ligand binds and/or unbinds with the aptamer, wherein the measurements are indicative of one or more characteristics of the ligand, and thereby characterize the ligand.

The methods may involve measuring one, two, three, four or five or characteristics of each ligand. Examples of suitable characteristics include, but are not limited to, (a) the length of the ligand (e.g., in base pairs or nucleotides), (b) the identity of the ligand, (c) the sequence of the ligand, (d) the secondary structure of the ligand; (e) whether or not the ligand is modified; or any combination thereof. In an embodiment, the method comprises an adapter-free method of characterization.

For (a), the length of the ligand may be measured for example by determining the number of interactions between the ligand and the aptamer and/or the nanopore or the duration of interaction between the ligand and/or the aptamer and/or the nanopore.

For (b) and (c), the identity of the ligand may be measured in any suitable way. The identity of the ligand may be measured in conjunction with measurement of the sequence of the ligand or without measurement of the sequence of the ligand. The sequencing of the ligand may comprise (i) applying an electric field to a nanopore system described herein; (ii) measuring an ion current generated by the electric field as each unit of the ligand individually passes through the nanopore to provide a current pattern that is associated with each unit, and (iii) comparing each current pattern to the current pattern of a known unit obtained under the same conditions, such that the ligand is sequenced. In an embodiment, the method further comprises a step of permitting each unit of the ligand to bind and/or unbind with an aptamer suspended in the lumen of the nanopore, wherein the binding and/or unbinding creates a measurable blockade in the current pattern. It will be appreciated that the methods are not limited to electric fields, but include other fields and forces as known in the art and described herein. When the ligand is not being sequenced, the presence of a particular motif in the ligand may be measured (without measuring the remaining sequence of the ligand). Alternatively, the measurement of a particular electrical and/or optical signal in the method may identify the ligand as coming from a particular source.

For (d), the secondary structure may be measured using any suitable method. For example, if the method involves an electrical measurement, the secondary structure may be measured using a change in dwell time or a change in current flowing through the pore. This allows different regions/motifs to be distinguished.

For (e), the presence or absence of any modification may be measured. The method preferably comprises determining whether or not the ligand is modified by some mechanism, including by methylation, by oxidation, by damage, with one or more small molecules or with one or more labels, tags or spacers. Specific modifications will result in specific interactions with the aptamer and/or nanopore which can be measured using the methods described herein. For instance, methylcyotsine may be distinguished from cytosine on the basis of the current flowing through the pore during its interaction with each nucleotide.

The methods may be carried out using any apparatus that is suitable for investigating the nanopore systems described herein.

The methods of the disclosure may involve the measuring of a current passing through the pore as the polynucleotide moves with respect to the pore. Suitable conditions for measuring ionic currents through transmembrane protein pores are known in the art and disclosed in the Examples. The method is typically carried out with a voltage applied across the membrane and nanopore. The voltage used is typically from +5 V to −5 V, such as from +4 V to −4 V, +3 V to −3 V or +2 V to −2 V. The voltage used is typically from −600 mV to +600 mV or −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The methods are typically carried out in the presence of any charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. As described herein, one or more salt solutions are present in the chambers. Potassium chloride (KCl), sodium chloride (NaCl), cesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a ligand to be identified against the background of normal current fluctuations.

The methods are typically carried out in the presence of a buffer. Any buffer may be used in the method of the disclosure. Typically, the buffer is phosphate buffer. Other suitable buffers are HEPES and Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5.

The methods may be carried out at any suitable temperature, for example from 0 °C to 100 °C, from 15 °C to 95 °C, from 16 °C to 90 °C, from 17 °C to 85 °C, from 18 °C to 80 °C, 19 °C to 70 °C, or from 20 °C to 60 °C. The methods may also be carried out at room temperature.

### Methods of Aptamer Motif Screening and Assessing Aptamer-Ligand Interactions

Methods of aptamer motif screening are provided. The methods of aptamer motif screening for ligand binding are useful for a variety of purposes, such as gene switch design for programming cellular functions by in vitro screening for ligand induced conformation changes. The methods of assessing aptamer-ligand interactions, also referred to as nucleic acid-small molecule interactions, have a variety of uses as well, for example, screening for small molecule regulators or potential therapeutic agents targeting nucleic acid motifs. Small molecules are regulators of various nucleic acid structures and functions, such as riboswitch aptamers, HIV trans-activation response (TAR) elements, Hepatitis C virus (HCV) internal ribosome entry site, SARS-CoV-2 frameshifting element, human microRNA and RNA repeats.

According to the methods described herein, the aptamer is docked within a nucleic acid-docked nanopore such that the aptamer is suspended within the lumen of the nanopore, preferably the center of the lumen. More particularly, the method of aptamer motif screening comprises (a) applying an ion current to a nucleic acid-docked nanopore; (b) providing an aptamer variant comprising a motif, (c) delivering the aptamer variant to the nanopore; (c) docking the aptamer variant in the nanopore through non-covalent binding between the aptamer variant and one or more nucleic acids in the nanopore, thereby suspending the aptamer variant in the nanopore; (d) delivering a ligand to the nanopore; (e) allowing the ligand to bind and/or unbind with the aptamer variant; (f) measuring the ion current as the binding and/or unbinding occurs to provide a current pattern that is associated the binding of the aptamer variant and ligand, and a current pattern that is associated with the unbinding of the aptamer variant and ligand; and (g) comparing the current patterns, wherein the current patterns are indicative of one or more characteristics of the aptamer variant; and (h) repeating steps (a)-(g) one or more times with a different aptamer variant.

The method of assessing nucleic acid-small molecule interactions comprises (a) applying an ion current to a nucleic acid-docked nanopore; (b) providing an aptamer; (c) delivering the aptamer to the nanopore; (c) docking the aptamer in the nanopore through non-covalent binding between the aptamer and one or more nucleic acids in the nanopore, thereby suspending the aptamer in the nanopore; (d) delivering a ligand to the nanopore; (e) allowing the ligand to bind and/or unbind with the aptamer; (f) measuring the ion current as the binding and/or unbinding occurs to provide a current pattern that is associated the binding of the aptamer and ligand, and a current pattern that is associated with the unbinding of the aptamer and ligand; and (g) comparing the current patterns, wherein the current patterns are indicative of one or more characteristics of the aptamer and/or the ligand.

In an embodiment, the one or more characteristics comprise the presence or absence of a binding event, the duration of a binding event, or a combination thereof. In an embodiment, the presence of a long-duration, single-level blocking event/binding event is indicative of a stable variant and/or desirable aptamer motif. The methods may involve measuring one, two, three, four or five or more variants of each aptamer. In an embodiment, the method comprises an adapter-free method of screening.

### Kits

It should be understood that the systems, methods, compositions, and apparatuses described herein may be incorporated into a kit or a device, such as a sensing or diagnostic device capable of receiving and analyzing a sample.

Any of the embodiments discussed above with reference to the methods, systems, compositions, and apparatuses of the disclosure equally apply to the kits or devices.

The kit or device may additionally comprise one or more other reagents or instruments which enable any of the embodiments described herein to be carried out, to process a sample input, and/or to provide an output (such as a report). Such reagents or instruments include one or more of the following: suitable buffer(s) (aqueous solutions), means to obtain a sample from a subject (such as a vessel or an instrument comprising a needle), means to amplify and/or express polynucleotides, a membrane as defined above or voltage or patch clamp apparatus. Reagents may be present in the kit or device in a dry state such that a fluid sample resuspends the reagents. The kit or device may also, optionally, comprise instructions to enable the kit or device to be used in the method of the disclosure or details regarding for which organism the method may be used.

The kit or device may be capable of receiving and analyzing a sample and providing an output regarding the same. One or more ligands may be present in any suitable sample. The sample may be a biological sample. The methods disclosed herein may be carried out in vitro using at least one sample obtained from or extracted from any organism or microorganism. The organism or microorganism may comprise an archaeal, prokaryotic or eukaryotic organism. The methods may be carried out and the kit or device may be used in vitro on at least one sample obtained from or extracted from any virus. The sample is preferably a fluid sample, for example the sample may comprise a body fluid of an individual. The sample may be urine, lymph, saliva, mucus, amniotic fluid, blood, plasma or serum. The sample may be human in origin, but alternatively it may be from another animal such as from livestock animals such as horses, cattle, sheep, fish, chickens or pigs or pets such as cats or dogs. Alternatively, the sample may be of plant origin, such as a sample obtained from a commercial crop, such as a cereal, legume, fruit or vegetable, for example wheat, barley, oats, canola, maize, soya, rice, rhubarb, bananas, apples, tomatoes, potatoes, grapes, tobacco, beans, lentils, sugar cane, cocoa, or cotton.

The sample may be a non-biological sample, including but not limited to, surgical fluids, water such as drinking water, sea water, river water, or a reagent for laboratory tests.

The sample is typically processed prior to being subjected to the methods, systems, compositions, and apparatuses described herein, for example by centrifugation or by passage through a membrane that filters out unwanted molecules or cells, such as red blood cells. The sample is optionally processed by a component in the kit or device. The sample may be measured immediately upon being taken or the sample may have been stabilized and/or stored before measurement.

### EXAMPLES

### Example 1. Preparation of the MspA and Variant Proteins

Engineered MspA, MspA-M2, is a widely studied protein nanopore for sequencing and other biomolecular detections. M2 substitutes six negatively charged amino acids (D and E) in the lumen of the wildtype MspA by neutral polar (N) and positively charged amino acids (R and K), specifically, D90N/D91N/D93N/D118R/D134R/E139K. MspA-M2 was as the model nanopore to study aptamer/ligand interactions and used MspA-M2 as the background to construct variants at the R118 and R134 sites to probe the aptamer docking mechanism. The three M2-based variants are M2-R118N/R134N, M2-R118N and M2-R134N. Internally, the three variants are named M3, M8 and M7.

The proteins of M2 and its variants were prepared as described in Yan, et al., Direct Sequencing of 2′-Deoxy-2′-Fluoroarabinonucleic Acid (Fana) Using Nanopore-Induced Phase-Shift Sequencing (Nipss). Chemical Science 2019, 10, 3110-3117; Wang, et al., Osmosis-Driven Motion-Type Modulation of Biological Nanopores for Parallel Optical Nucleic Acid Sensing. ACS Appl Mater Interfaces 2018, 10, 7788-7797; Heinz, et al., High-Level Expression of the Mycobacterial Porin MspA in Escherichia coli and Purification of the Recombinant Protein. J Chromatogr B Analyt Technol Biomed Life Sci 2003, 790, 337-348 and Butler, et al., Single-Molecule DNA Detection with an Engineered MspA Protein Nanopore. Proc Natl Acad Sci USA 2008, 105, 20647-20652, each of which are herein incorporated by reference in their entirety.

In particular, the genes of M2 and variants with poly-histidine tag (H6) were inserted into the plasmid pET-30a(+) and cloned by GenScript Inc. Competent cells (E. coli BL21 (DE3)) were transformed with the plasmids by heat shock and then plated on LB agar supplemented with 50 μg/ml kanamycin. The plates were incubated at 37 °C overnight. A single colony was picked and grown in 3 ml LB medium with 50 μg/ml kanamycin and then was sub-cultured in 200 ml same medium. When OD600=0.7-1.0, the cells were induced by 1 mM isopropyl β-D-thiogalactoside (IPTG) and shaken overnight at 16 °C. They were harvested by centrifugation at 4000 rpm for 30 min at 4 °C in centrifuge tubes. The supernatant was discarded, and cell pellets were lysed in the lysis buffer (100 mM Na₂HPO₄/NaH₂PO₄, 0.1 mM EDTA, 150 mM NaCl, 0.5% (w/v) Genapol X-80 pH 6.5) at 60 °C for 10 min. The lysed cells were kept on ice for 10 min and centrifuged at 10,000 rpm 30 min at 4 °C. After syringe filtration through a 0.22 μm filter, the supernatant was transferred to a nickel affinity column (HisTrap™ HP, GE Healthcare).

After washing the column by washing buffer (0.5 M NaCl, 20 mM HEPES, 5 mM imidazole, 0.5% (w/v) Genapol X-80, pH=8.0), the MspA mutants were eluted by using the elution buffer (500 mM imidazole, 0.5 M NaCl, 20 mM HEPES, 0.5% (w/v) Genapol X-80, pH=8.0). The elution aliquots (0.3-0.5 ml) with a gradient concentration of imidazole were sequentially collected in EP tubes. The assembly of MspA mutants was characterized by 12% SDS-PAGE. The aliquots with octamers were selected for the nanopore recording.

### Example 2. Aptamers and Small-Molecule Ligands

The dopamine- and serotonin-binding aptamers prepared as described in Nakatsuka, et al., Aptamer-Field-Effect Transistors Overcome Debye Length Limitations for Small-Molecule Sensing. Science 2018, 362, 319-324 and the theophylline RNA aptamer was prepared as reported Jenison et a., High-Resolution Molecular Discrimination by RNA. Science 1994, 263, 1425-1429 and Zimmermann et al., Molecular interactions and metal binding in the theophylline-binding core of an RNA aptamer. RNA. 2000, 6, 659-67, each of which are herein incorporated by reference in their entirety. The respective K_{d} for ligands binding to their aptamers is shown in the table below.

TABLE 1
K_{d} for ligands binding to their aptamers measured
by nanopore and reported in the literature

| | Nanopore | Literatures |
|---|---|---|
| Dopamine Aptamer | 27 ± 3 μM | 0.18 μM |
| Serotonin Aptamer | 9.0 ± 1.3 μM | 0.03 μM |
| Theophylline Aptamer | 72 ± 17 μM | 0.1-0.2 μM |

The sequences of the three aptamers and a group of dopamine aptamer variants used are provided in the table below and correspond to SEQ ID NO:1 for the dopamine-binding aptamer, SEQ ID NO:2 for the serotonin-binding aptamer, SEQ ID NO:3 for the theophylline-binding aptamer, SEQ ID NO:9 for ΔL1/L2, SEQ ID NO:10 for ΔL1, SEQ ID NO:11 for ΔL2, and SEQ ID NO:12 for GG>GA.

TABLE 2
Sequences of Aptamers and Variants

| | |
|---|---|
| | 1------10-------20------30------40---------50 |
| Dopamine- | CGACGCCAGTTTGAAGGTTCGTTCGCAGGTGTGGAGtGACGTCG |
| binding aptamer | |
| ΔL1/L2 | CGACGCCAGT------------------------AGTGACGTCG |
| ΔL1 | CGACGCCAGTTTG------------CAGGTGTGGAGTGACGTCG |
| ΔL2 | CGACGCCAGTTTGAAGGTTCGTTCG--------GAGTGACGTCG |
| GG > GA | CGACGCCAGTTTGAAGATTCGTTCGCAGATGTGAAGTGACGTCG |
| Serotonin- | CGACTGGTAGGCAGATAGGGGAAGCTGATTCGATGCGTGGGTCG |
| binding aptamer | |
| Theophylline- | AGUGAUACCAGCAUCGUCUUGAUGCCCUUGGCAGCACU |
| binding aptamer | |

All the DNA and RNA fragments were synthesized by Integrated DNA Technologies, Inc. They were dissolved in deionized water to 1 mM and diluted to 100 μM in 100 mM KCl, 20 mM Tris-Cl, pH 8.0 as the stock. RNAase-free water (New England Biolab) was used for RNA preparation. Before using in nanopore detection, aptamers were denatured at 95 °C for 2 min, followed by cooling down gradually to room temperature overnight. Small molecule ligands, including dopamine, serotonin, norepinephrine, and theophylline, were purchased from Sigma Inc.

### Example 3. Nanopore Single-Channel Recording

Nanopore single-channel recordings were conducted as described in Wang et al., Nanopore-Based Detection of Circulating Micrornas in Lung Cancer Patients. Nat Nanotechnol 2011, 6, 668-674 and Tian et al., Single Locked Nucleic Acid-Enhanced Nanopore Genetic Discrimination of Pathogenic Serotypes and Cancer Driver Mutations. ACS Nano 2018, 12, 4194-4205, each of which are herein incorporated by reference in their entirety.

In particular, the lipid bilayer membrane (1,2-diphytanoyl-sn-glycero-3-phosphocholine, Avanti Polar Lipids) was formed over a 100-150 μm orifice in the center of the Teflon film that partitioned between cis and trans recording solutions. The solutions in both cis and trans chambers contained 1 M KCl buffered with 10 mM Tris (pH 7.4). In the RNA aptamer experiments, the solution also contained 5 mM MgCl₂. The MspA proteins were added to the cis solution, from which they were inserted into the bilayer to form a single nanopore channel. Each aptamer was added to the cis solution at 100 nM, and each small molecule ligand was added to the trans solution at the desired concentrations. The voltage was applied from the trans solution, and the cis solution was the reference (ground). The nanopore ion currents for all the experiments were recorded using an Axopatch 200B amplifier (Molecular Device Inc., Sunnyvale, CA), filtered with a built-in 4-pole low-pass Bessel filter at 5 kHz (bandwidth), and acquired with Clampex 10 (Molecular Device Inc.) through a Digidata 1440 A/D converter (Molecular Device Inc.) at a sampling rate of 20 kHz. In selected experiments, the nanopore currents were also recorded at the 100 kHz bandwidth and acquired at a 400 kHz sampling rate, followed by software filtering at 20 kHz and/or 10 kHz with Clampfit 10. The nanopore experiments were performed at 22±2 °C. Each measurement was an individual average from N independently reconstituted nanopores, N≥3. The result was presented as mean±s.d., where s.d. is standard deviation.

### Example 4. Nanopore Current Trace Analysis

Nanopore current trace analyses, including event duration histogram analysis and amplitude histogram analysis, were conducted using Clampfit 9/10 (Molecular Device Inc.), Excel (Microsoft) and SigmaPlot (SPSS) software. The aptamer residence time in the nanopore (τ_{A}) and the duration of the ligand-binding events (τ_{off}) follow an exponential distribution. This distribution was binned in the log(t) scale in the histograms and was fit in Clampfit. The elapsed time between two consecutive ligand binding events, i.e., the duration from the end of one ligand-binding event to the beginning of the next ligand binding event (τₒₙ), was fit by an exponential distribution. The current amplitude of each blocking level was obtained by fitting the peaks in all-point histograms to a Gaussian distribution in Clampfit.

The standard deviations of the nanopore ion currents for the free and ligand-bound aptamer conformations (I_{SD}, as shown in FIG. 6A) was determined as follows: for each aptamer conformation, 10 current segments were randomly selected from a nanopore recording to calculate the standard deviation of all data points in each segment. The mean of these segment standard deviations represents I_{SD} of the recording. I_{SD} from three recordings (N=3) were measured, and their mean and standard deviation (SD of I_{SD}) were calculated and illustrated in FIG. 6B and Table 2. I_{SD} values from different recordings for the ligand-free (A) and ligand-bound (AL) aptamer conformations were evaluated with the t-test, with p indicating the probability of the null hypothesis.

TABLE 3
Standard deviation of nanopore current (ISD) for the
free and ligand-bound aptamer conformations
I_{SD} ± SD (pA)

| | Dopamine- | Serotonin- | Theophylline- |
|---|---|---|---|
| | binding | binding | binding |
| | aptamer | aptamer | aptamer |
| Aptamer (A) | 12.2 ± 0.65 | 2.72 ± 0.32 | 3.54 ± 0.05 |
| Aptamer•Ligand (AL) | 0.84 ± 0.03 | 2.28 ± 0.03 | 1.66 ± 0.1 |
| t-test | p = 0.000004 | p = 0.04 | p = 0.000005 |

### Example 5. Kinetics for the Dopamine-Binding Aptamer Conformational Change in the Absence and in the Presence of Dopamine: Aptamer in the Absence of Dopamine

As demonstrated in FIG. 21, the expanded nanopore current signatures show that the dopamine aptamer residing in the M2 nanopore rapidly transitions among multiple conformations identified from their distinct blocking levels, including A₁ and A₂, and intermediate clusters A_{Int} (shaded intervals) that mediate A₁/A₂ transitions. The kinetic scheme of aptamer conformation changes can be simplified as A₁↔A_{Int}↔A₂, described by rate constant k_{A1} for the transition A₁→A_{Int}, k_{A1}′ for the transition A_{Int}↔A₁, k_{A2} for the transition A₂→A_{Int}, and k_{A2}′ for the transition A_{Int}→A₂. From the durations of A₁ (τ_{A1}) and A₂ (τ_{A2}), the following can be obtained:

k_{A1}=1/τ_{A1} (S1)

k_{A2}=1/τ_{A2} (S2)

For transitions from Δ_{Int} to A₁ and A₂, the sum of the rate constants k_{A1}′ and k_{A2}′ can be calculated from the duration of A_{Int} (τ_{AInt}), i.e.,

k_{A1}′+k_{A2}′=1/τ_{AInt} (S3)

From the number of the A_{Int}→A₁ transitions (N_{A1}) and the number of A_{Int}→A₂ transitions (N_{A2}) in a recording, the relative frequency of transitions can be calculated as

P_{A1}=N_{A1}/(N_{A1}+N_{A2}) P_{A2}=N_{A2}/(N_{A1}+N_{A2})

Finally, from τ_{AInt}, P_{A1} and P_{A2}, k_{A1}′ and k_{A2}′can be calculated as

k_{A1}′=(k_{A1}′+k_{A2}′) P_{A1}=P_{A1}/τ_{AI} (S4)

k_{A2}′=(k_{A1}′+k_{A2}′) P_{A2}=P_{A2}/τ_{AI} (S5)

It has been measured that

τ_{A1}=1±2 ms,

τ_{A2}=8.2±1.3 ms

τ_{AInt}=7.3±1.8 ms.

It was also measured that P_{A1}=0.54 P_{A2}=0.46

As a result, the rate constants for aptamer conformation transitions were calculated to be

k_{A1}=91±19 s⁻¹

k_{A1}′=122±21 s⁻¹

k_{A2}=75±17 s⁻¹ k_{A2}′=64±17 s⁻¹

### Example 6. Kinetics for the Dopamine-Binding Aptamer Conformational Change in the Absence and in the Presence of Dopamine: Aptamer in the Presence of Dopamine

As shown in FIG. 22, the overall aptamer signature is a recording of a sequence of dynamic transitions between the free aptamer A, consisting of states A₁, A₂ and A_{Int}, and dopamine-bound aptamer AL. The A→AL transition is characterized by the apparent associate rate constant kₒₙ and the release of dopamine from AL is characterized by the dissociate rate constant k_{off}. For the association process,

kₒₙ=f/[L]=1/(τ_{off}·[L]) (S7)

Where τₒₙ is the elapsed time between consecutive AL blocks, the frequency of the A→AL transitions or the frequency of the dopamine binding events f=1/τₒₙ. For the dissociation process,

k_{off}=1/τ_{off} (S8)

### Example 7. Detection of Small Molecule Binding to Nucleic Acid Motifs

To validate the nanopore's ability to detect interactions between small molecules and nucleic acid motifs, crucial for nucleic acid-targeted therapeutic discovery, the MspA-M2 pore was utilized to capture the binding of the anti-cancer small molecule Mitoxantrone (MTX) to both DNA and RNA motifs.

In FIG. 23A, for DNA motif, a 28-base pair double-stranded DNA (dsDNA) molecule (SEQ ID NO: 13) was inserted into the MspA-M2 pore from the cis side. Under conditions of 150 mV and 1 M KCl (pH 7.4), the docked DNA exhibited a single-level current state, Level-1, with a blocking level of I/I₀=0.233±0.008 and a duration of τ_{A}=0.14±0.02 s. In FIG. 23B, upon introducing 100 μM of Mitoxantrone (MTX) on the trans side of the nanopore, a series of consecutive Level-2 states immediately emerged within each Level-1 state. These Level-2 states had a blocking level of I/I₀=0.118±0.010, a duration of τ_{off}=4.1±0.9 ms, and occurred at a frequency of f=160±20 s⁻¹. These newly observed Level-2 states corresponded to an MTX-bound DNA conformation. Thus, the continuous recording of Level-2 states represented individual MTX molecule interactions with the DNA, yielding an association rate constant kₒₙ=1.6±0.2×10⁶ M⁻¹·s⁻¹ (where kₒₙ=f/[MTX]) and a dissociation rate constant k_{off}250±60 s⁻¹ (calculated as 1/τ_{off}), with an apparent equilibrium dissociation constant K_{D}=160 μM. Additionally, the presence of MTX increased the DNA lifetime within the pore by approximately 8-fold, extending from τ_{A}=0.14±0.02 s without MTX to 1.2±0.3 s with MTX, highlighting MTX's role in stabilizing dsDNA.

The Human Immunodeficiency Virus Type 1 (HIV-1) Trans-activator Response (TAR) RNA served as the RNA motif for investigating MTX binding. In FIG. 24A and FIG. 24C, when a TAR RNA molecule was inserted into the MspA-M7 nanopore from the cis entrance in 1 M at 160 mV in 1 M KCl (pH7.4), it produced two distinct nanopore current states capable of transitioning between each other. These states corresponded to the TAR RNA transitioning between two conformational states: low-noise Level-A1, characterized by a low standard deviation (I_{SD}=7.1 pA) and a higher blocking level (I/I₀=0.520), and high-noise Level-B1, marked by a high standard deviation (I_{SD}=13.3 pA) and a lower blocking level (I/I₀=0.449). In FIG. 24B and FIG. 24D, upon introducing 100 μM of MTX into the trans solution of the nanopore, a series of consecutive short upward current states, i.e., Level-A2 from Level-A1 (ΔI=3 pA, τ_{off}=8.0 ms) and Level-B2 from Level-B1 (ΔI=3 pA, τ_{off}=8.0 ms), were immediately observed. These new Level-A2 and Level-B2 states resulted from the binding of individual MTX molecules to both conformational states of TAR RNA, likely at the bulge as indicated by the nuclear magnetic resonance (NMR) studies. Compared to MTX binding to DNA, MTX binding to TAR RNA induced upward signals that were significantly shorter in duration, suggesting distinct molecular mechanisms for MTX binding to DNA versus RNA motifs.

In conclusion, this example confirms the efficacy of nanopores in detecting small molecule binding to nucleic acid motifs. This validation underscores the nanopore's capability to record conformational state changes in these motifs upon small molecule binding, thus highlighting its potential for applications in nucleic acid-targeted therapeutic discovery.

1: A nucleic acid-docked nanopore system comprising:
(a) a phospholipid membrane;
(b) a mutant Mycobacterium smegmatis porin A (MspA) nanopore transversing the membrane and
comprising a cis vestibule and a trans vestibule, wherein the cis vestibule comprises a lumen and the trans vestibule comprises a constriction; wherein the mutant MspA comprises a mutation at positions 90, 91, 93, 118, 134, and/or 139, wherein a negatively charged amino acid at positions 90, 91, 93, 118, 134, and/or 139 of a wild-type MspA is replaced by a positively charged amino acid or a neutral polar amino acid such that the one or more positively charged amino acids or a neutral polar amino acids are distributed around the interior circumference of the mutant MspA nanopore thereby forming a ring;
(c) an aptamer non-covalently bound with the ring on the interior surface of the mutant MspA nanopore such that the aptamer is suspended in the lumen via non-covalent bonding between the aptamer and the nanopore; wherein non-covalent binding suspends the aptamer in the ring;
(d) a ligand capable of binding with the aptamer;
(e) an electrical circuit capable of applying an electric field to the nanopore system and generating an ion current, wherein one or more measurements are taken of the ion current; wherein the one or more measurements are indicative of one or more characteristics of the aptamer, the ligand, an interaction between the aptamer and the ligand, or an interaction between the aptamer and the nanopore;
wherein the nanopore system provides real-time characterization of the aptamer, the ligand, the interaction between the aptamer and the ligand, or the interaction between the aptamer and the nanopore; and
wherein the nanopore system is adapter-free.

2: The nucleic acid-docked nanopore system of claim 1, further comprising: (f) a cis chamber located on the side of the membrane closest to the cis vestibule; a (g) a trans chamber located the side of the membrane closest to the trans vestibule; and/or (h) an apparatus capable of taking one or more measurements of the ion current.

3: The nucleic acid-docked nanopore system of claim 1, wherein the mutant MspA comprises a mutant as shown in one or more of SEQ ID NO: 7, SEQ ID NO: 4, SEQ ID NO: 6, and SEQ ID NO: 5.

4: The nucleic acid-docked nanopore system claim 1, wherein the ligand comprises a neurotransmitter, antibody, receptor, peptide, nucleic acid sequence, hormone, metabolite, antibiotic, therapeutic compound, biomarker, and/or diagnostic compound.

5: The nucleic acid-docked nanopore system of claim 1, wherein the aptamer comprises a sequence of DNA, RNA, or XNA, a peptide, oligonucleotide, a riboswitch aptamer, an RNA element, an RNA structure, an RNA entry site, a frameshifting element, and/or RNA repeats.

6: The nucleic acid-docked nanopore system of claim 5, wherein the aptamer is an oligonucleotide having the sequence shown in SEQ ID NO: 1 and has the ability to bind with a ligand comprising dopamine; the aptamer is an oligonucleotide having the sequence shown in SEQ ID NO: 2 and has the ability to bind with a ligand comprising serotonin; and/or the aptamer is an oligonucleotide having the sequence shown in SEQ ID NO: 3 and has the ability to bind with a ligand comprising theophylline.

7: A method of non-covalently docking an aptamer in a nucleic acid-docked nanopore comprising:
(a) providing a mutant Mycobacterium smegmatis porin A (MspA) nanopore comprising a cis vestibule comprising a lumen and a trans vestibule comprising a constriction that define a channel, wherein the mutant MspA comprises one or more mutations at positions that are distributed around the interior circumference of the mutant MspA nanopore thereby forming a ring;
(b) delivering an aptamer to the mutant MspA nanopore; and
(c) non-covalently binding the aptamer with the ring on the interior surface of the mutant MspA nanopore, wherein non-covalent binding suspends the aptamer in the ring;
wherein neither the nanopore or aptamer are modified with an adapter and wherein the aptamer is capable of binding with a ligand that is not modified with an adapter.

8: The method of claim 7, wherein the mutant MspA comprises a mutant as shown in one or more of SEQ ID NO: 7, SEQ ID NO: 4, SEQ ID NO: 6, and SEQ ID NO: 5.

9: The method of claim 7, wherein the aptamer comprises a sequence of DNA, RNA, or XNA, a peptide, oligonucleotide, a riboswitch aptamer, an RNA element, an RNA structure, an RNA entry site, a frameshifting element, and/or RNA repeats.

10: The method of claim 9, wherein the aptamer is an oligonucleotide comprising a nucleobase sequence according to any one or more of SEQ ID NO: 1, SEQ ID NO: 2, and SEQ ID NO: 3.

11: A method of ligand characterization comprising:
(a) providing an inlaid nucleic acid nanopore system comprising (i) a phospholipid membrane;
(ii) a mutant Mycobacterium smegmatis porin A (MspA) nanopore transversing the membrane and comprising a cis vestibule and a trans vestibule, wherein the cis vestibule comprises a lumen and the trans vestibule comprises a constriction; wherein the mutant MspA comprises a mutation at positions 90, 91, 93, 118, 134, and/or 139, wherein a negatively charged amino acid at positions 90, 91, 93, 118, 134, and/or 139 of a wild-type MspA is replaced by a positively charged amino acid or a neutral polar amino acid such that the one or more positively charged amino acids or a neutral polar amino acids are distributed around the interior circumference of the mutant MspA nanopore thereby forming a ring; (iii) an aptamer non-covalently bound with the ring on the interior surface of the mutant MspA nanopore such that the aptamer is suspended in the lumen via non-covalent bonding between the aptamer and the nanopore; wherein non-covalent binding suspends the aptamer in the ring; (iv) a ligand capable of binding with the aptamer; (v) an electrical circuit capable of applying an electric field; wherein the nanopore system is adapter-free;
(b) applying an electric field to the nanopore system using the electrical circuit, thereby generating an ion current;
(c) delivering the ligand to the nanopore such that the ligand moves into the pore;
(d) allowing the ligand to bind and/or unbind with the aptamer; and
(e) taking one or more measurements of the ion current;
wherein the one or more measurements are indicative of one or more characteristics of the aptamer, the ligand, an interaction between the aptamer and the ligand, or an interaction between the aptamer and the nanopore; and
wherein the nanopore system provides real-time characterization of the aptamer, the ligand, the interaction between the aptamer and the ligand, or the interaction between the aptamer and the nanopore.

12: The method of claim 11, wherein the one or more characteristics comprise the length of the ligand, the identity of the ligand, the sequence of the ligand, the presence of the ligand, the absence of the ligand, the secondary structure of the ligand; whether or not the ligand is modified, the conformation of the aptamer, the dwell time of the ligand; the blocking level of the aptamer, the blocking duration, the block occurrence; or a combination thereof.

13: The method of claim 12, wherein the length of the ligand is measured by sequencing the ligand.

14: The method of claim 13, wherein the sequencing comprises measuring the ion current generated by the electric field as each unit of the ligand individually binds and unbinds with the aptamer to provide measurable blockade in a current pattern that is associated with each unit; and comparing the current pattern to a current pattern of a known unit obtained under the same conditions, such that the ligand is sequenced.

15: The method of any one of claim 11, wherein the aptamer is an aptamer variant comprising a motif, and wherein the motif comprises a hairpin, a single-branched loop, a multi-branched loop, a helix, a bulge, or a G-quadruplex.

16: The method of claim 15, wherein the one or more characteristics comprises the blocking level of the aptamer.

17: The method of claim 15, wherein the method further comprises aptamer variant screening; and wherein the aptamer variant screening occurs by repeating the method one or more times with a different aptamer variant.

18: The method of claim 11, wherein the one or more characteristics comprises the presence of a long-duration, single-level blocking event and/or the absence of a long-duration, single-level blocking event; and wherein the presence of a long-duration, single-level block event is indicative of a stable aptamer-ligand interaction.

19: The method of claim 11, wherein the presence of the ligand is indicative of a phenotype or with a type of cell.

20: The method of claim 19, wherein the phenotype comprises a disease or a medical condition; and wherein the cell comprises a bacterium, a virus, a fungus, or a parasite.
